(19) United States
(12) Patent Application Publication
(10) Pub. No.: US 2010/0035919 A1
(43) Pub. Date: Feb. 11, 2010

(54) COMPOUNDS USEFUL AS INHIBITORS OF PROTEIN KINASES

(75) Inventors: Anil Vasudevan, Union Grove, WI (US); Brian S. Brown, Evanston, IL (US); Ryan G. Keddy, Beach Park, IL (US); Adrian D. Hobson, Shrewsbury, MA (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/534,432

(22) Filed: Aug. 3, 2009

Related U.S. Application Data
(60) Provisional application No. 61/086,279, filed on Aug. 5, 2008.

(51) Int. Cl. A61K 31/437; C07D 471/02

(52) U.S. Cl. 514/300; 546/113

(57) ABSTRACT

Disclosed herein are compounds of formula (I) or pharmaceutical acceptable salts thereof,

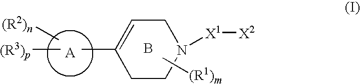

wherein A, X¹, X², R¹, R², R³, m, n, and p are defined in the specification. Compositions including the compounds which can be useful for inhibiting Rho kinase (ROCK) and methods for using the compositions are also described.

This application claims priority to U.S. patent application Ser. No. 61/086,279, filed Aug. 5, 2008, and is incorporated herein by reference.

### TECHNICAL FIELD

Bicyclic compounds that are inhibitors of Rho kinases (ROCK), compositions including such compounds, and methods for treating conditions and disorders using such compounds and compositions are provided.

### BACKGROUND

An important large family of enzymes is the protein kinase enzyme family. Currently, there are about 500 different known protein kinases. Protein kinases serve to catalyze the phosphorylation of an amino acid side chain in various proteins by the transfer of the y-phosphate of the ATP-Mg²⁺ complex to the amino acid side chain.

These enzymes control the majority of the signalling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase can itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied families of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP-and cyclic GMP-dependent protein kinases, calcium- and phospholipid-dependent protein kinase, calcium-and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases.

Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also in progress to identify modulators of tyrosine kinases as well.

A major signal transduction system utilized by cells is the RhoA-signalling pathway. RhoA is a small GTP binding protein that can be activated by several extracellular stimuli such as growth factor, hormones, mechanic stress, or osmotic change as well as high concentration of metabolite like glucose. RhoA activation involves GTP binding, conformation alteration, post-translational modification (geranylization and farnesylation) and activation of its intrinsic GTPase activity. Activated RhoA is capable of interacting with several effector proteins including ROCKs (Rho kinase) and transmit signals into cellular cytoplasm and nucleus.

Rho kinase is found in two isoforms encoded by two different genes of ROCK, ROCK 1 (also known as ROCKβ or p160-ROCK) and ROCK 2 (also known as ROCKα). Both ROCK 1 and ROCK 2 contain an amino-terminal catalytic kinase domain, a central coiled-coil domain of about 600 amino acids, and a carboxyl-terminal pleckstrin homology (PH) domain that is split by a cysteine-rich region. Rho/GTP interacts with the C-terminal portion of the central coiled-coil domain and activates the kinase activity of ROCK.

Thus, ROCK1 and 2 constitute a family of serine/threonine kinases that can be activated by RhoA-GTP complex via physical association. Activated ROCKs phosphorylate a number of substrates and play important roles in pivotal cellular functions. The substrates for ROCKs include myosin binding subunit of myosin light chain phosphatase (MBS, also named MYPT1), adducin, moesin, myosin light chain (MLC), LIM kinase as well as transcription factor FHL. The phosphorylation of theses substrates modulate the biological activity of the proteins and thus provide a means to alter cell's response to external stimuli. One well documented example is the participation of ROCK in smooth muscle contraction. Upon stimulation by phenylephrine, smooth muscle from blood vessels contracts. Studies have shown that phenylephrine stimulates alpha-adrenergic receptors and leads to the activation of RhoA. Activated RhoA in turn stimulates kinase activity of ROCK1 and which in turn phosphorylates MBS. Such phosphorylation inhibits the enzyme activity of myosin light chain phosphatase and increases the phosphorylation of myosin light chain itself by a calcium-dependent myosin light chain kinase (MLCK) and consequently increases the contractility of myosin-actin bundle, leading to smooth muscle contraction. This phenomenon is also sometimes called calcium sensitization. In addition to smooth muscle contraction, ROCKs have also been shown to be involved in cellular functions including apoptosis, cell migration, transcriptional activation, fibrosis, cytokinesis, inflammation and cell proliferation. Moreover, in neurons ROCK plays a critical role in the inhibition of axonal growth by myelin-associated inhibitory factors such as myelin-associated glycoprotein (MAG). ROCK-activity also mediates the collapse of growth cones in developing neurons. Both processes are thought to be mediated by ROCK-induced phosphorylation of substrates such as LIM kinase and myosin light chain phosphatase, resulting in increased contractility of the neuronal actin-myosin system.

Abnormal activation of the Rho/ROCK pathway has been observed in various disorders (Wettschureck, N., Offermanns, S., Rho/Rho-kinase mediated signaling in physiology and pathophysiology. J. Mol. Med. 80, 2002, 629-638; Müller, B. K., Mack, H., Teusch, N., Rho kinase, a promising drug target for neurological disorders. Nat. Drug Discov. Rev. 4, 2005, 387-398; Hu, E, Lee, D., ROCK inhibitors as potential therapeutic agents for cardiovascular diseases. Curr. Opin. Investig. Drugs. 4, 2003, 1065-1075). As already mentioned, ROCKs phosphorylate the myosin binding subunit of myosin light chain (MLC) phosphatase (MLCP), resulting in increased myosin phosphorylation and actin-myosin contraction (Somlyo, A. P., Somlyo, A. V., Ca2+ sensitivity of smooth muscle and nonmuscle myosin II: modulated by G proteins, kinases, and myosin phosphatase. Physiol. Rev. 83, 2003, 1325-1358). Examples of disease states related with abnormal Rho/ROCK activity, in particular with vasospasm activity, include cardiovascular diseases such as hypertension (Satoh S., Kreutz R., Wilm C., Ganten D., Pfitzer G., Augmented agonist-induced Ca²⁺-sensitization of coronary artery contraction in genetically hypertensive rats. Evidence for altered signal transduction in the coronary smooth muscle cells. J. Clin. Invest. 94, 1994, 1397-1403; Mukai, Y., Shimokawa, H., Matoba, T., Kandabashi, T., Satoh, S., Hiroki, J., Kaibuchi, K., Takeshita, A., Involvement of Rho-kinase in hypertensive vascular disease: a novel therapeutic target in hypertension. FASEB J. 15, 2001,1062-1064; Uehata, M., Ishizaki, T., Satoh, H., Ono, T., Kawahara, T., Morishita, T., Tamakawa, H., Yamagami, K., Inui, J., Maekawa, M., Narumiya, S., Calcium sensitization of smooth muscle mediated by a Rho-associated protein kinase in hypertension. Nature 389, 1997, 990-994; Masumoto, A., Hirooka, Y., Shimokawa, H., Hironaga, K., Setoguchi, S., Takeshita, A., Possible involvement of Rhokinase in the pathogenesis of hypertension in humans. Hypertension 38, 2001, 1307-1310), chronic and congestive heart failure (Fuster, V., Badimon, L., Badimon, J J, Chesebro, J H, The pathogenesis of coronary artery disease and the acute coronary syndromes (2). N Engl J Med 326, 1992, 310-318; Shimokawa, H., Cellular and molecular mechanisms of coronary artery spasm: lessons from animal models. Jpn Circ J 64, 2000, 1-12; Shimokawa, H., Morishige, K., Miyata, K., Kandabashi, T., Eto, Y., Ikegaki, I., Asano, T., Kaibuchi, K., Takeshita, A., Longterm inhibition of Rho-kinase induces a regression of arteriosclerotic coronary lesions in a porcine model in vivo. Cardiovasc Res 51, 2001, 169-177; Utsunomiya, T., Satoh, S., Ikegaki, I., Toshima, Y., Asano, T., Shimokawa, H., Antianginal effects of hydroxyfasudil, a Rho-kinase inhibitor, in a canine model of effort angina. Br J Pharmacol 134, 201, 1724-1730), cardiac hypertrophy (Hoshijima, M., Sah, V. P., Wang, Y., Chien, K. R., Brown, J. H., The low molecular weight GTPase Rho regulates myofibril formation and organization in neonatal rat ventricular myocytes. Involvement of Rho kinase. J Biol Chem 273, 1998, 7725-77230; Sah, V. P., Hoshijima, M., Chien, K. R., Brown, J. H., Rho is required for Galphaq and alpha1-adrenergic receptor signal-637 ing in cardiomyocytes. Dissociation of Ras and Rho pathways. J Biol Chem 271, 1996, 31185-1190; Kuwahara, K., Saito, Y., Nakagawa, O., Kishimoto, I., Harada, M., Ogawa, E., Miyamoto, Y., Hamanaka, I., Kajiyama, N., Takahashi, N., Izumi, T., Kawakami, R., Tamura, N., Ogawa, Y., Nakao, K., The effects of the selective ROCK inhibitor, Y27632, on ET-1-induced hypertrophic response in neonatal rat cardiacmyocytes-possible involvement of Rho/ROCK pathway in cardiac muscle cell hypertrophy. FEBS Lett 452, 1999, 314-318), chronic renal failure (Sharpe, C. C., Hendry, B., M. Signaling: focus on Rho in renal disease. J. Am. Soc. Nephrol. 14, 2003, 261-264), cerebral vasospasm after subarachnoid bleeding (Shibuya, M., Suzuki, Y., Sugita, K., Saito, I., Sasaki, T., Takakura, K., Okamoto, S., Kikuchi, H., Takemae, T., Hidaka, H., Dose escalation trial of a novel calcium antagonist, AT877, in patients 636 with aneurysmal subarachnoid haemorrhage. Acta Neurochir (Wien) 107, 1990, 11-15; Shibuya, M., Suzuki, Y., Sugita, K., Saito, I., Sasaki, T., Takakura, K., Nagata, I., Kikuchi, H., Takemae, T., Hidaka, H., et. al, Effect of AT877 on cerebral vasospasm after aneurysmal subarachnoid hemorrhage. Results of a prospective placebo-controlled double-blind trial. J Neurosurg 76, 1992, 571-577; Sato, M., Tani, E., Fujikawa, H., Kaibuchi, K., Involvement of Rho-kinase-mediated phosphorylation of myosin light chain in enhancement of cerebral vasospasm. Circ Res 87, 2000, 195-200; Miyagi, Y., Carpenter, R. C., Meguro, T., Parent, A. D., Zhang, J. H., Upregulation of rho A and rho kinase messenger RNAs in the basilar artery of a rat model of subarachnoid hemorrhage. J Neurosurg 93, 2000, 471-476; Tachibana, E., Harada, T., Shibuya, M. Saito, K., Takayasu, M., Suzuki, Y., Yoshida, J., Intra-arterial infusion of fasudil hydrochloride for treating vasospasm following subarachnoid hemorrhage. Acta Neurochir (Wien) 141, 1999, 13-19), pulmonary hypertension (Sylvester, J. T., Am. J. Physiol. Lung Cell. Mol. Physiol. 287, 2004, L624-L630) and ocular hypertension (Honjo, M., Inatani, M., Kido, N., Sawamura, T., Yue, B. Y., Honda, Y., Tanihara, H., Effects of protein kinase inhibitor, HA1077, on intraocular pressure and outflow facility in rabbit eyes. Arch Ophthalmol 119, 2001, 1171-1178; Rao, P. V, Deng, P. F., Kumar, J. Epstein, D. L., Modulation of aqueous humor outflow facility by the Rho kinase-specific inhibitor Y-27632. Invest Ophthalmol Vis Sci 42, 2001, 1029-1037). Further diseases related to abnormal Rho/ROCK activity are cancer (Aznar, S., Fernandez-Valeron, P., Espina, C., Lacal, J. C., and Rho GTPases: potential candidates for anticancer therapy. Cancer Lett. 206, 2004, 181-191; Yin, L. et al., Fasudil inhibits vascular endothelial growth factor-induced angiogenesis in vitro and in vivo. Mol Cancer Ther 5, 2007, 1517-25; Itoh, K., Yoshioka, K., Akedo, H., Uehata, M., Ishizaki, T., Narumiya, S., An essential part for Rho-associated kinase in the transcellular invasion of tumor cells. Nat Med 5, 1999, 221-225; Genda, T. Sakamoto, M., Ichida, T., Asakura, H., Kojiro, M., Narumiya, S., Hirohashi, S., Cell motility mediated by rho and Rho-associated protein kinase plays a critical role intrahepatic metastasis of human hepatocellular carcinoma. Hepatology 30, 1999, 1027-1036; Somlyo, A. V., Bradshaw, D., Ramos, S., Murphy, C., Myers, C. E., Somlyo, A. P., Rho-kinase inhibitor retards migration and in vivo dissemination of human prostate cancer cells. Biochem Biophys Res Commun 269, 2000, 652-659), asthma (Roberts, J. A., Raeburn, D., Rodger, I. W., Thomson, N. C., Comparison of in vivo airway responsiveness and in vitro smooth muscle sensitivity to methacholine in man. Thorax 39; 1984, 837-843; Chiba, Y., Misawa, M., Characteristics of muscarinic cholinoceptors in airways of antigen-induced airway hyperresponsive rats. Comp Biochem Physiol C Pharmacol Toxicol Endocrinol 111, 1995, 351-357; Chiba, Y., Takada, Y., Miyamoto, S., MitsuiSaito, M., Karaki, H., Misawa, M., Augmented acetylcholine-induced, Rho mediated Ca²⁺ sensitization of bronchial smooth muscle contraction in antigen-induced airway hyperresponsive rats. Br J Pharmacol 127, 1999, 597-600; Chiba, Y., Sakai, H. Misawa, M., Augmented acetylcholine-induced translocation of RhoA in bronchial smooth muscle from antigen-induced airway hyperresponsive rats. Br J Pharmacol 133, 2001, 886-890; Tizuka, K., Shimizu, Y., Tsukagoshi, H., Yoshii, A., Harada, T. Dobashi, K., Murozono, T., Nakazawa, T., Mori, M., Evaluation of Y-27632, a rho-kinase inhibitor, as a bronchodilator in guinea pigs. Eur J Pharmacol 406, 2000, 273-279), male erectile dysfunctions (Andersson, K. E., Hedlund, P., New directions for erectile dysfunction therapies. Int. J. Impot. Res. 14 (Suppl. 1 ), 2002, S82-S92; Chitaley, K., Wingard, C. J., Clinton Webb, R., Branam, H., Stopper, V. S., Lewis, R. W., Mills, T. M., Antagonism of Rho-kinase stimulates rat penile erection via a nitric oxide independent pathway. Nat Med 7, 2001, 119-122; Mills, T. M., Chitaley, K., Wingard, C. J., Lewis, R. W., Webb, R. C., Effect of Rho-kinase inhibition on vasoconstriction in the penile circulation. J Appl Physiol 91, 2001, 1269-1273), female sexual dysfunction, over-active bladder syndrome (Peters, S. L. et al., Rho kinase: a target for treating urinary bladder dysfunction. Trends Pharmacol Sci. 27, 2006, 492-7) and preterm labor (Niiro, N., Nishimura, J., Sakihara, C., Nakano, H., Kanaide, H., Up-regulation of rho A and rho-kinase mRNAs in the rat myometrium during pregnancy. Biochem Biophys Res Commun 230, 1997, 356-359; Tahara, M., Morishige, K., Sawada, K., Ikebuchi, Y., Kawagishi, R., Tasaka, K., Murata, Y., RhoA/Rho-kinase cascade is involved in oxytocin-induced rat uterine contraction. Endocrinology 143, 2002, 920-929; Kupittayanant, S., Burdyga, T., Wray, S., The effects of inhibiting Rho-associated kinase with Y-27632 on force and intracellular calcium in human myometrium. Pflugers Arch. 443, 2001, 112-114).

Inhibitors of ROCKs have been suggested for use in the treatments of a variety of diseases. They include cardiovascular diseases such as hypertension, chronic and congestive heart failure, and cardiac hypertrophy, chronic renal failure, furthermore cerebral vasospasm after subarachnoid bleeding, pulmonary hypertension, and ocular hypertension. In addition, because of their muscle relaxing properties, they are also suitable for asthma, male erectile dysfunctions, female sexual dysfunction and over-active bladder syndrome, and preterm labor. Several recent studies have reported the beneficial effects of ROCK inhibitors in ischemia-reperfusion and myocardial infarction. In these studies, the ROCK inhibitors Y-27632 and fasudil were shown to decrease ischemia-reperfusion injury, myocardial infarct size, and myocardial fibrosis in response to experimental myocardial infarction (MI) and in a rat model of chronic hypertension induced congestive heart failure (Masumoto, A., Mohri, M., Shimokaw, a H., Urakami, L., Usui, M., Takeshita, A., Suppression of coronary artery spasm by the rho-kinase inhibitor fasudil in patients with vasospastic angina. Circulation 105, 2002, 1545-1547; Shimokawa, H., Iinuma, H., Kishida, H., et al., Antianginal effect of fasudil, a Rho-kinase inhibitor, in patients with stable effort angina: a multicenter study (abstract). Circulation 104[Suppl II], 2001, 11691; Morishige K, Shimokawa H, Eto Y, Kandabashi T, Miyata K, Matsumoto Y, Hoshijima M, Kaibuchi K, Takeshita A, Adenovirus-mediated transfer of dominant-negative rho-kinase induces a regression of coronary arteriosclerosis in pigs in vivo. Arterioscler Thromb Vasc Biol 21, 2001, 548-554; Kandabashi T, Shimokawa H, Mukai Y, Matoba T, Kunihiro I, Morikawa K, Ito M, Takahashi S, Kaibuchi K, Takeshita A, Involvement of rho-kinase in agonists-induced contractions of arteriosclerotic human arteries. Arterioscler Thromb Vasc Biol 22, 2002, 243-248; Liu M W, Roubin G S, King S B 3^{rd}, Restenosis after coronary angioplasty. Potential biologic determinants and role of intimal hyperplasia. Circulation 79, 1989, 1374-1387; Shibata R, Kai H, Seki Y, Kato S, Morimatsu M, Kaibuchi K, Imaizumi T, Role of Rho-associated kinase in neointima formation after vascular injury. Circulation 103, 2001, 284-289).

Additionally, ROCKs can interact with other signalling pathways resulting in inhibition of phosphoinositide-3 kinase (PI-3K), endothelial nitric oxide synthase (eNOS) pathways, and activation of plasminogen activator inhibitor-1 (PAI-1) which can contribute to endothelial dysfunction like restenosis and atherosclerosis. Thus ROCK inhibitors have been suggested for the treatment of restenosis and atherosclerosis (Iwasaki, H. et al., High glucose induces plasminogen activator inhibitor-1 expression through Rho/Rho-kinase-mediated NF-kappaB activation in bovine aortic endothelial cells. Atherosclerosis, 2007, Jan. 31).

Vascular intimal thickening in vein grafts after surgery is the major cause of late graft failure. In a study with the ROCK inhibitor fasudil, the intimal thickening and vascular smooth muscle cell (VSMC) proliferation was significantly suppressed, whereas VSMC apoptosis was enhanced in the weeks following the procedure, suggesting that ROCK inhibitors can be used as a therapeutic agent for the prevention of graft failure.

Injury to the adult vertebrate brain and spinal cord activates ROCKs, thereby causing neurodegeneration and inhibition of neuroregeneration like neurite growth and sprouting (Bito, H., Furuyashiki, T., Ishihara, H., Shibasaki, Y., Ohashi, K., Mizuno, K., Maekawa, M., Ishizaki, T., Narumiya, S., A critical role for a Rho-associated kinase, p160ROCK, in determining axon outgrowth in mammalian CNS neurons. Neuron 26, 2000, 431-441). Inhibition of ROCKs results in induction of new axonal growth, axonal rewiring across lesions within the CNS, accelerated regeneration and enhanced functional recovery after acute neuronal injury in mammals (spinal-cord injury, traumatic brain injury) (Hara, M. et al., Protein kinase inhibition by fasudil hydrochloride promotes neurological recovery after spinal cord injury in rats. J. Neurosurg. Spine 93, 94-101; Fournier, A. E., Takizawa, B. T. & Strittmatter, S. M., ROCK inhibition enhances axonal regeneration in the injured CNS. J. Neurosci. 23, 2003, 1416-1423; Sung, J. K. et al., A possible role of RhoA/Rho-kinase in experimental spinal cord injury in rat. Brain Res. 959, 2003, 29-38; Tanaka, H. et al., Cytoplasmic p21 (Cip1/WAF1) enhances axonal regeneration and functional recovery after spinal cord injury in rats. Neuroscience 127, 2004, 155-164). ROCK inhibitors are therefore likely to be useful for regenerative (recovery) treatment of CNS disorders such as spinal cord injury, acute neuronal injury (stroke, traumatic brain injury) (Okamura N et al., Vasodilator effects of fasudil, a Rho-kinase inhibitor, on retinal arterioles in stroke-prone spontaneously hypertensive rats. J Ocul Pharmacol Ther. 23, 2007, 207-12; Yagita Y et al., Rho-kinase activation in endothelial cells contributes to expansion of infarction after focal cerebral ischemia. J Neurosci Res. 85, 2007, 2460-9), Parkinson's disease, Alzheimer disease (Pedrini S et al., Modulation of statin-activated shedding of Alzheimer APP ectodomain by ROCK. PLoS Med.2, 2005, 18; Burton A., NSAIDS and Alzheimer's disease: it's only Rock and Rho. Lancet Neurol. 3(1), 2004, 6) and other neurodegenerative disorders. Other neurodegenerative disorders for which ROCK inhibitors are expected to be useful are Huntington's disease (Shao J, Welch W J, Diprospero N A, Diamond M I. Phosphorylation of profilin by ROCK1 regulates polyglutamine aggregation. Mol Cell Biol. 2008 September; 28(17):5196-208; Shao J, Welch W J, Diamond M I. ROCK and PRK-2 mediate the inhibitory effect of Y-27632 on polyglutamine aggregation. FEBS Lett. 2008 May 28; 582(12):1637-42), spinal muscular atrophy (Bowerman M, Shafey D, Kothary R. Smn depletion alters profilin II expression and leads to up regulation of the RhoA/ROCK pathway and defects in neuronal integrity. J Mol Neurosci. 2007; 32(2): 120-31) and amyotrophic lateral sclerosis. Inhibition of the Rho/ROCK pathway has also proved to be efficacious in other animal models of neurodegeneration like stroke and in inflammatory and demyelinating diseases like multiple sclerosis (Sun X et al., The selective Rho-kinase inhibitor Fasudil is protective and therapeutic in experimental autoimmune encephalomyelitis. J Neuroimmunol. 180, 2006, 126-34), acute and chronic pain (Inoue, M. et al., Initiation of neuropathic pain requires lysophosphatidic acid receptor signaling. Nature Med. 10, 2004, 712-718; Ramer, L. M., Borisoff, J. F. & Ramer, M. S., Rho-kinase inhibition enhances axonal plasticity and attenuates cold hyperalgesia after dorsal rhizotomy. J Neurosci. 24, 2004, 10796-10805; Tatsumi, S. et al., Involvement of Rho-kinase in inflammatory and neuropathic pain through phosphorylation of myristoylated alanine-rich C-kinase substrate (MARCKS). Neuroscience 131, 2005, 491-498).

ROCK inhibitors have been shown to possess anti-inflammatory properties by decreased cytokine release, e.g. TNFα. Thus they can be used as treatment for neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, osteoarthritis, osteoporosis, asthma, irritable bowel syndrome, or inflammatory bowel disease (Segain J. P., Rho kinase blockade prevents inflammation via nuclear factor kappa B inhibition: evidence in Crohn's disease and experimental colitis. Gastroenterology. 124(5), 2003, 1180-7). In addition, recent reports have demonstrated that inhibition of ROCK results in disruption of inflammatory cell chemotaxis as well as inhibition of smooth muscle contraction in models of pulmonary inflammation associated with asthma. Therefore, the inhibitors of the Rho/ROCK pathway should be useful for the treatment of asthma (Kawaguchi A, Ohmori M, Harada K, Tsuruoka S, Sugimoto K, Fujimura A., The effect of a Rho kinase inhibitor Y-27632 on superoxide production, aggregation and adhesion inhuman polymorphonuclear leukocytes. Eur J Pharmacol 403, 2000, 203-208 ; Lou Z, Billadeau D D, Savoy D N, Schoon R A, Leibson P. J., A role for a RhoA/ROCK/LIM-kinase pathway in the regulation of cytotoxic lymphocytes. J Immunol 167, 2001, 5749-5757; Vicente-Manzanares M, Cabrero J R, Rey M, Perez-Martinez M, Ursa A, Itoh K, Sanchez-Madrid F., A role for the Rho-p160 Rho coiled-coil kinase axis in the chemokine stromal cell-derived factor-1alpha-induced lymphocyte actomyosinand microtubular organization and chemotaxis. J Immunol 168, 2002, 400-410; Thorlacius K et al., Protective effect of fasudil, a Rho-kinase inhibitor, on chemokine expression, leukocyte recruitment, and hepatocellular apoptosis in septic liver injury. J Leukoc Biol. 79, 2006, 923-31).

Since ROCK inhibitors reduce cell proliferation and cell migration, they could be useful in treating cancer and tumor metastasis. ROCK inhibitors can also be beneficial in diseases with impaired blood brain barrier function, e.g. HIV-1 encephalitis (Persidski Y et al., Rho-mediated regulation of tight junctions during monocyte migration across the blood-brain barrier in HIV-1 encephalitis (HIVE). Blood. 107, 2006, 4770-4780) and Alzheimer's disease (Man S-M et al., Peripheral T cells over express MIP-1a to enhance its transendothelial migration in Alzheimer's disease. Neurobiol. Of Aging 28, 2007, 485-496).

Furthermore, there is evidence suggesting that ROCK inhibitors suppress cytoskeletal rearrangement upon virus invasion, thus they also have potential therapeutic value in anti-viral and anti-bacterial applications (Favoreel H W, Cytoskeletal rearrangements and cell extensions induced by the US3 kinase of an alphaherpesvirus are associated with enhanced spread. Proc Natl Acad Sci USA. 102(25), 2006, 8990-5).

ROCKs have been reported to interfere with insulin signalling through serine phosphorylation of insulin receptor substrate-1 (IRS-1), in cultured VSMC. Activation of RhoA/ROCK was observed in skeletal muscles and aortic tissues of Zucker obese rats. Inhibition of ROCK, by fasudil for 4 weeks, reduced blood pressure, corrected glucose and lipid metabolism, improved insulin signalling and endothelial dysfunction. In another experiment administration of high dose fasudil completely suppressed the development of diabetes, obesity, and dyslipidemia and increased serum adiponectin levels in OLETF rats. ROCK inhibitors can therefore be useful for the treatment of insulin resistance and diabetes (Nakamura Y et al., Marked increase of insulin gene transcription by suppression of the Rho/Rho-kinase pathway. Biochem Biophys Res Commun. 350(i), 2006, 68-73; ⁶⁶Kikuchi Y et al., A Rho-kinase inhibitor, fasudil, prevents development of diabetes and nephropathy in insulin-resistant diabetic rats. J Endocrinol. 192(3), 2007, 595-603; Goyo A et al., the Rho-kinase inhibitor, fasudil, attenuates diabetic nephropathy in streptozotocin-induced diabetic rats. Eur J Pharmacol. 568(1-3), 2007, 242-7).

The ROCK inhibitor Fasudil increased cerebral blood flow and was neuroprotective under CNS ischemic conditions. ROCK inhibitors are expected to be useful for the treatment of ischemic CNS disorders and can therefore improve functional outcome in patients suffering from stroke, vascular or AD type dementia.

Due to the efficacy of Y-27632 and fasudil in animal models of epileptogenesis, ROCK inhibitors have been suggested for the use in the treatments of epilepsy and seizure disorders (Inan S Y, Büyükafsar K. Antiepileptic effects of two Rho-kinase inhibitors, Y-27632 and fasudil, in mice. Br. J. Pharmacol. advance online publication, 9 Jun. 2008; doi:10.1038/bjp.2008.225)

ROCK inhibitors are also expected to be useful for the treatment of glaucoma, psoriasis, retinopathy and benign prostatic hypertrophy.

Furthermore, there is evidence suggesting that ROCK inhibitors suppress cytoskeletal rearrangement upon virus invasion, thus they also have potential therapeutic value in anti-viral and anti-bacterial applications.

As ROCKs have been implicated in neuronal morphogenesis, connectivity, and plasticity in general, they are expected to be useful for the treatment of psychiatric disorders, e.g. depression, schizophrenia, obsessive compulsive disorder and bipolar disorders.

ROCK inhibitors have been described in e.g. WO 2007/026920, WO 2005/074643, and WO 2004/016597. However, their affinity and selectivity or their pharmacological profile is not yet satisfactory.

### SUMMARY OF THE INVENTION

Generally provided herein are bicyclic compounds that are Rho kinases inhibitors, pharmaceutical compositions including such compounds, and methods for the treatment of disorders using these compounds and pharmaceutical compositions.

Generally, the present invention is directed towards compounds of formula (I), or pharmaceutically acceptable salts, solvates, prodrugs, salts of prodrugs, or combinations thereof,

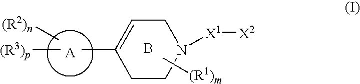

wherein

R¹ represents optional substituent(s) on ring B, and each occurrence of R¹ is independently alkyl, CN, —O(R^{1a}), —N(R^{1b})(R^{1c}), —(C₁₋₆ alkylenyl)-O(R^{1a}), —(C₁₋₆ alkylene)-N(R^{1b})(R^{1c}), —(C₁₋₆ alkylene)-CN, alkenyl, halogen, or haloalkyl;

R^{1a} and R^{1b}, at each occurrence, are each independently hydrogen, alkyl or haloalkyl;

R^{1c}, at each occurrence, is independently hydrogen, alkyl, haloalkyl, O(R^{za}), C(O)NR^{za}R^{zb}, C(O)R^{zb}, S(O)₂R^{zc}, or S(O)₂NR^{za}R^{zb}; wherein each occurrence of R^{za} and R^{zb} are each independently hydrogen, alkyl or haloalkyl, and R^{zc} is alkyl or haloalkyl;

R² represents optional substituent(s) on the carbon atom(s) of ring A, and each occurrence of R² is independently aryl, heteroaryl, cycloalkyl, cycloalkenyl, heterocycle, arylalkyl, heteroarylalkyl, cycloalkylalkyl, cycloalkenylalkyl, or heterocyclealkyl; wherein each of the aryl, heteroaryl, cycloalkyl, cycloalkenyl, and heterocycle moieties, as a substituent or part of a substituent, is independently unsubstituted or substituted with 1, 2, 3, 4, or 5 substituents as represented by R^{7a};

R³ represents optional substituent(s) on the carbon atom(s) of ring A;

m is 0, 1, 2, or 3;

n is 0 or 1;

p is 0, 1, 2, or 3;

A is formula (i), (ii), or (iii)

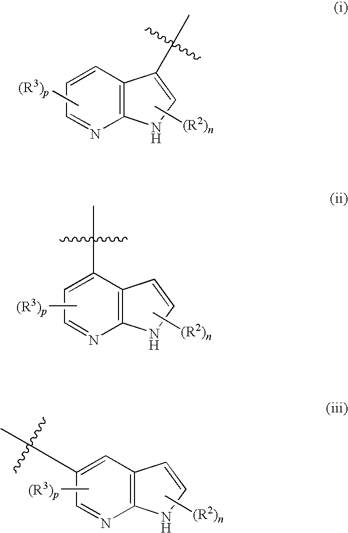

wherein

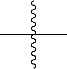

represents the point of connection to ring B; and R² and R³ are optional substituents on any substitutable carbon atoms within the bicyclic ring;

X¹ is C(O), C(S), C(O)O, C(O)N(R⁴), S(O), S(O)₂, S(O)₂N(R⁴), or C(═NR⁵); wherein the C(O)O, C(O)N(R⁴), and the S(O)₂N(R⁴) are connected to the nitrogen atom of ring B through the carbon and the sulfur atoms respectively; and

x² is hydroxyalkyl, —(CR^{6a}R^{6b})_{q}-G¹, -alkenylene-G¹, —(CR^{6a}R^{6b})ᵣ—X³-G¹, —(CR^{6a}R^{6b})_{q}—X³—(CR^{6a}R^{6b})_{q}-G¹, or J^{A} wherein
X³ is O, S, N(H), or N(alkyl);
G¹ at each occurrence, is independently cycloalkyl, cycloalkenyl, heterocycle, heteroaryl, or aryl, each of which is independently unsubstituted or substituted with 1, 2, 3, 4, or 5 substituents as represented by R^{7b};
J^{A} is a monocyclic heterocycle or a monocyclic cycloalkyl optionally substituted with 1, 2, 3, 4, 5, or 6 substituents as represented by R^{7JA}; two R^{7JA} on the adjacent carbon atoms of J^{A}, together with the carbon atoms to which they are attached, optionally form a benzo, a monocyclic heterocycle, a monocyclic cycloalkyl, or a monocyclic cycloalkenyl ring wherein each of the rings is independently unsubstituted or substituted with 1, 2, or 3 substituents as represented by R^{7b};
R^{6a} and R^{6b} can be the same or different, and at each occurrence, are each independently hydrogen, halogen, haloalkyl, aryl, —OR^{u}, —N(R^{v})(R^{w}), or alkyl; wherein the alkyl is optionally substituted with one substituent selected from the group consisting of —OR^{u}, —N(R^{v})(R^{w}), aryl, and monocyclic heterocycle; wherein the aryl group and the monocyclic heterocycle group are each independently unsubstituted or substituted with 1, 2, 3, 4, or 5 substituents as represented by R⁶za;

or

X¹-X² together is a five membered monocyclic heterocycle or a five membered monocyclic heteroaryl ring, optionally substituted with 1, 2, 3, or 4 substituents as represented by R^{7c};

R⁴ is hydrogen or alkyl which is optionally substituted with 1 or 2 substituents independently selected from the group consisting of OH, O(alkyl), halogen, —C(O)(alkyl), —C(O)O(alkyl), —C(O)NH₂, —C(O)N(H)(alkyl), —C(O)N(alkyl)₂, cycloalkyl, cycloalkenyl, heterocycle, aryl, and heteroaryl;

R^{u}, R^{v}, and R^{w}, at each occurrence, are each independently hydrogen, alkyl, or haloalkyl;

R^{7JA} and R^{7c}, at each occurrence, are each independently alkyl, alkenyl, alkynyl, halogen, oxo, NO₂, CN, haloalkyl, OR^{a}, OC(O)R^{a}, NR^{a}R^{b}, N(R^{b})C(O)R^{a}, N(R^{b})S(O)₂R^{a}, SR^{a}, S(O)R^{c}, S(O)₂R^{c}, S(O)₂NR^{a}R^{b}, C(O)R^{a}, C(O)OR^{a}, C(O)NR^{a}R^{b}, —(C₁₋₆alkylene)-NO₂, —(C₁₋₆ alkylene)-CN, —(C₁₋₆ alkylene)-OR^{a}, —(C₁₋₆ alkylene)-OC(O)R^{a}, —(C₁₋₆ alkylene)-NR^{a}R^{b}, —(C₁₋₆ alkylene)-N(R^{b})C(O)R^{a}, —(C₁₋₆ alkylene)-N(R^{b})S(O)₂R^{a}, —(C₁₋₆ alkylene)-SR^{a}, —(C₁₋₆ alkylene)-S(O)R^{c}, —(C₁₋₆ alkylene)-S(O)₂R^{c}, —(C₁₋₆ alkylene)-S(O)₂NR^{a}R^{b}, —(C₁₋₆ alkylene)-C(O)R^{a}, —(C₁₋₆ alkylene)-C(O)OR^{a}, —(C₁₋₆ alkylene)-C(O)NR^{a}R^{b}, G², —(C₁₋₆ alkylene)-G², or —O(CR^{ax}R^{bx})ₜO— wherein the oxygen atoms of —O(CR^{ax}R^{bx})ₜO— are connected to the adjacent carbon atoms of the phenyl group;

R^{7b}, at each occurrence, is independently alkyl, alkenyl, alkynyl, halogen, oxo, NO₂, CN, haloalkyl, OR^{7ab}, OC(O)R^{7ab}, NR^{7ab}R^{b}, N(R^{b})C(O)R^{7ab}, N(R^{b})S(O)₂R^{7ab}, SR^{7ab}, S(O)R^{c}, S(O)₂R^{c}, S(O)₂NR^{7ab}R^{b}, C(O)R^{7ab}, C(O)OR^{7ab}, C(O)NR^{7ab}R^{b}, —(C₁₋₆ alkylene)-NO₂, —(C₁₋₆ alkylene)-CN, —(C₁₋₆ alkylene)-OR^{7ab}, —(C₁₋₆ alkylene)-OC(O)R^{7ab}, —(C₁₋₆ alkylene)-NR^{7ab}R^{b}, —(C₁₋₆ alkylene)-N(R^{b})C(O)R^{7ab}, —(C₁₋₆ alkylene)-N(R^{b})S(O)₂R^{7ab}, —(C₁₋₆ alkylene)-SR^{7ab}, —(C₁₋₆ alkylene)-S(O)R^{c}, —(C₁₋₆ alkylene)-S(O)₂R^{c}, —(C₁₋₆ alkylene)-S(O)₂NR^{7ab}R^{b}, —(C₁₋₆ alkylene)-C(O)R^{7ab}, -(C₁₋₆ alkylene)-C(O)OR^{7ab}, —(C₁₋₆ alkylene)-C(O)NR^{7ab}R^{b}, G², —(C₁₋₆ alkylene)-G², or —O(CR^{ax}R^{bx})ₜO— wherein the oxygen atoms of —O(CR^{ax}R^{bx})ₜO— are connected to the adjacent carbon atoms of the phenyl group;

G², at each occurrence, is independently cycloalkyl, cycloalkenyl, heterocycle, heteroaryl, or aryl, each of which is independently unsubstituted or substituted with 1, 2, 3, 4, or 5 substituents as represented by R^{7d};

R³, R^{7a}, R^{6za}, and R^{7d}, at each occurrence, are each independently alkyl, alkenyl, alkynyl, halogen, NO₂, CN, haloalkyl, OR^{a}, OC(O)R^{a}, NR^{a}R^{b}, N(R^{b})C(O)R^{a}, N(R^{b})S(O)₂R^{a}, SR^{a}, S(O)R^{c}, S(O)₂R^{c}, S(O)₂NR^{a}R^{b}, C(O)R^{a}, C(O)OR^{a}, C(O)NR^{a}R^{b}, —(C₁₋₆ alkylene)-NO₂, —(C₁₋₆ alkylene)-CN, —(C₁₋₆ alkylene)-OR^{a}, —(C₁₋₆ alkylene)-OC(O)R^{a}, —(C₁₋₆ alkylene)-NR^{a}R^{b}, —(C₁₋₆ alkylene)-N(R^{b})C(O)R^{a}, —(C₁₋₆ alkylene)-N(R^{b})S(O)₂R^{a}, —(C₁₋₆ alkylene)-SR^{a}, —(C₁₋₆ alkylene)-S(O)R^{c}, —(C₁₋₆ alkylene)-S(O)₂R^{c}, —(C₁₋₆ alkylene)-S(O)₂NR^{a}R^{b}, —(C₁₋₆ alkylene)-C(O)R^{a}, —(C¹⁻⁶ alkylene)-C(O)OR^{a}, or —(C₁₋₆ alkylene)-C(O)NR^{a}R^{b};

R^{a} and R^{b}, at each occurrence, are each independently hydrogen, alkyl, or haloalkyl;

R^{ax} and R^{bx}, at each occurrence, are each independently hydrogen, halogen, alkyl, or haloalkyl;

R^{7ab}, at each occurrence, is independently hydrogen, alkyl, haloalkyl, G², or —(C₁₋₆ alkylene)-G²;

R^{c}, at each occurrence, is independently alkyl or haloalkyl;

q, at each occurrence, is independently 1, 2, 3, or 4;

t is 1,2, or 3; and

r is 2, 3, or 4;

with the proviso that

(a) when A is formula (i), X¹ is C(O), and X² is -alkenylene-G¹, then G¹ is not monocyclic heteroaryl; and

(b) when A is formula (ii), X¹ is C(O), X² is —(CR^{6a}R^{6b})_{q}-G¹, and G¹ is aryl, then one of R^{6a} and R^{6b} is other than N(R^{v})(R^{w}).

Another aspect of the present invention relates to pharmaceutical compositions including therapeutically effective amounts of one or more compounds presented herein, or pharmaceutically acceptable salts or solvates thereof, in combination with one or more pharmaceutically acceptable carrier, adjuvants, excipients, or other auxiliary substances. These pharmaceutical compositions are useful for treating diseases as described herein.

The compounds of the present invention are useful for the prevention or treatment of diseases associated with abnormal ROCK activity. Thus, pharmaceutically effective compositions of such compounds or pharmaceutically acceptable salts or solvates thereof are useful for the prevention or treatment of the diseases.

The compounds of the present invention have inhibitory activity against ROCK-1 and ROCK-2 kinases and are thus useful for the inhibition of such kinases. Accordingly, the compounds or pharmaceutically acceptable salts or solvates thereof can be useful as active ingredients for the preparation of compositions, which enable preventive and/or therapeutic treatment of diseases or conditions caused by abnormal ROCK kinases (including ROCK-1 and ROCK-2) activity. The diseases which respond to the modulation of ROCKs, in particular to ROCKs inhibition include, but are not limited to, pain such as, but not limited to, neuropathtic pain, nociceptive pain, inflammatory pain, and cancer pain; cardiovascular diseases such as hypertension, chronic and congestive heart failure, cardiac hypertrophy, restenosis, chronic renal failure, atherosclerosis, asthma, male erectile dysfunctions, female sexual dysfunction, over-active bladder syndrome, neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, irritable bowel syndrome, or inflammatory bowel disease. In addition, based on their neurite outgrowth inducing effects, ROCK inhibitors can be used as drugs for neuronal regeneration, inducing new axonal growth and axonal rewiring across lesions within the CNS. ROCK inhibitors are therefore useful for regenerative (recovery) treatment of CNS disorders such as spinal cord injury, acute neuronal injury (stroke, traumatic brain injury), Parkinson's disease, Alzheimer disease and other neurodegenerative disorders, such as, in particular, Huntington's disease, spinal muscular atrophy, and amyotrophic lateral sclerosis. Since ROCK inhibitors reduce cell proliferation and cell migration, they could be useful in treating cancer and tumor metastasis. Furthermore, ROCK inhibitors suppress cytoskeletal rearrangement upon virus invasion and also have potential therapeutic value in anti-viral and anti-bacterial applications. ROCK inhibitors can also be useful for the treatment of insulin resistance and diabetes. ROCK inhibitors can furthermore be useful for the treatment of ischemic CNS disorders, vascular or AD type dementia, glaucoma, psoriasis, retinopathy, benign prostatic hypertrophy, psychiatric disorders, in particular depression, schizophrenia, obsessive compulsive disorder and bipolar disorder, epilepsy and seizure disorders, for decreasing ischemia-reperfusion injury, myocardial infarct size and myocardial fibrosis, and for the prevention of graft failure. Accordingly, the compounds described herein can be used for treating the above-listed disorders. More preferably, they are used for treating pain, asthma, Alzheimer's disease, multiple sclerosis, rheumatoid arthritis, and spinal cord injuries.

A further aspect provides methods of treating diseases as described herein above. The methods include administering to the subject (including human) in need thereof therapeutically effective amounts of one or more compounds described herein or pharmaceutically acceptable salts or solvates thereof, with or without one or more pharmaceutically acceptable carriers, excipients, adjuvants, or other auxiliary substances.

The present application further provides uses of compounds described herein or pharmaceutically acceptable salts or solvates thereof, with or without one or more pharmaceutically acceptable carriers, excipients, adjuvants, or other auxiliary substances, in the manufacture of medicaments for the treatment of the diseases or conditions described herein.

### DETAILED DESCRIPTION OF THE INVENTION

Compounds of formula (I) are disclosed

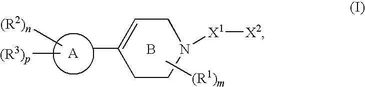

wherein A, X¹, X², R¹, R², R³, m, n, and p are as defined above in the Summary and below in the Detailed Description. Compositions including such compounds and methods for treating conditions and disorders using such compounds and compositions are also disclosed.

In various embodiments, one or more variable can occur more than one time in any substituent or in the compounds described or any other formulae herein. Definition of a variable on each occurrence is independent of its definition at another occurrence. Further, combinations of substituents or variables are permissible only if such combinations result in stable compounds. Stable compounds are compounds, which can be isolated from a reaction mixture.

### a. Definitions

As used in the specification and the appended claims, unless specified to the contrary, the following terms have the meaning indicated:

As used in the specification and the appended claims, unless specified to the contrary, the following terms have the meaning indicated:

The term “alkenyl” as used herein, means a straight or branched chain hydrocarbon containing from 2 to 10 carbons, for example, 2-6 carbons, and more preferably 2-4 carbons, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” or “alkenylenyl” denotes a divalent group derived from a straight or branched hydrocarbon chain of 2, 3, or 4 carbon atoms and contains at least one carbon-carbon double. Representative examples of alkenylene or alkenylenyl include, but are not limited to, —CH═CH— and —CH₂CH═CH—.

The term “alkyl” as used herein, means a saturated, straight or branched hydrocarbon chain containing from 1 to 10 carbon atoms, for example from 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 1-methylbutyl, 2-methylbutyl, 3-methylbutyl, 1,1-dimethylpropyl, 1,2-dimethylpropyl, 2,2-dimethylpropyl, 1-methylpropyl, 1-ethylpropyl, 1,2,2-trimethylpropyl, 3-methylhexyl, 2,2-dimethylpentyl, 2,3-dimethylpentyl, n-heptyl, n-octyl, n-nonyl, and n-decyl.

The term “alkylene” or “alkylenyl” means a divalent group derived from a saturated, straight or branched hydrocarbon chain of from 1 to 10 carbon atoms. The term “C₁₋₆ alkylene” means those alkylene or alkylenyl groups having from 1 to 6 carbon atoms. Representative examples of alkylene include, but are not limited to, —CH₂—, —CH(CH₃)—, —CH(C₂H₅), —CH(CH(CH₃)(C₂H₅))—, —C(H)(CH₃)CH₂CH₂—, —C(CH₃)₂—, —CH₂CH₂—, —CH₂CH₂CH₂—, —CH₂CH₂CH₂CH₂—, and —CH₂CH(CH₃)CH₂—.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 1,1-dimethylprop-2-ynyl, 1-propyl-pent-3-ynyl, 3-butynyl, 2-pentynyl, and 1-butynyl.

The term “aryl,” as used herein, means phenyl, a bicyclic aryl or a tricyclic aryl. The bicyclic aryl is naphthyl, or a phenyl fused to a monocyclic cycloalkyl, or a phenyl fused to a monocyclic cycloalkenyl. Non limiting examples of the bicyclic aryl include dihydroindenyl, indenyl, naphthyl, dihydronaphthalenyl, and tetrahydronaphthalenyl (including 1,2,3,4-tetrahydronaphthalen-1-yl). The tricyclic aryl is exemplified by a bicyclic aryl fused to a monocyclic cycloalkyl, or a bicyclic aryl fused to a monocyclic cycloalkenyl, or a bicyclic aryl fused to a phenyl. Non limiting examples of tricyclic aryls include anthracene, phenanthrene, dihydroanthracenyl, fluorenyl, 1,2-dihydroacenaphthylenyl, and tetrahydrophenanthrenyl. The phenyl, bicyclic and tricyclic aryls are attached to the parent molecular moiety through any carbon atom contained within the phenyl, bicyclic, and tricyclic aryls respectively.

The term “arylalkyl” as used herein, means an aryl group, as defined herein, appended to the parent molecular moiety through an alkylene or alkylenyl group, as defined herein. Non-limiting examples of arylalkyl include benzyl (phenylmethyl), naphthylmethyl and phenylethyl.

The term “cycloalkenyl” as used herein, means a monocyclic or bicyclic ring system containing zero heteroatoms in the ring. The monocyclic cycloalkenyl has three-, four-, five-, six-, seven- or eight carbon atoms and zero heteroatoms. The three or four-membered ring systems have one double bond, the five-or six-membered ring systems have one or two double bonds, and the seven- or eight-membered ring systems have one, two or three double bonds. Representative examples of monocyclic cycloalkenyls include, but are not limited to, cyclohex-1-en-1-yl, 2-cyclohexen-1-yl, 3-cyclohexen-1-yl, 2,4-cyclohexadien-1-yl and 3-cyclopenten-1-yl. Bicyclic cycloalkenyls are exemplified by a monocyclic cycloalkenyl fused to a monocyclic cycloalkyl, or a monocyclic cycloalkenyl fused to a monocyclic cycloalkenyl. Non limiting examples of bicyclic ring systems include 3a, 4, 5, 6, 7, 7a-hexahydro-1H-indenyl, 4,5,6,7-tetrahydro-3aH-indene, and octahydronaphthalenyl. The cycloalkenyl groups are appended to the parent molecular moiety through any substitutable carbon atom within the groups, and can contain one or two alkylene bridges of 1, 2, 3, or 4 carbon atoms, wherein each bridge links two non-adjacent atoms within the groups.

The term “cycloalkenylalkyl,” as used herein, means a cycloalkenyl group, as defined herein, appended to the parent molecular moiety through an alkylene or alkylenyl group, as defined herein.

The term “cycloalkyl” as used herein, means a monocyclic, or a bicyclic cycloalkyl, or a spirocyclic cycloalkyl. The monocyclic cycloalkyl is a carbocyclic ring system containing 3, 4, 5, 6, 7, or 8 carbon atoms and zero heteroatoms as ring atoms, and zero double bonds. Examples of monocyclic cycloalkyls include cyclopropyl, cyclobutyl, cyclopentyl, cyclohexyl, cycloheptyl, and cyclooctyl. Bicyclic cycloalkyl is exemplified by a monocyclic cycloalkyl fused to a monocyclic cycloalkyl. Non limiting examples of bicyclic cycloalkyls include bicyclo[4.1.0]heptane, bicyclo[6.1.0]nonane, octahydroindene, and decahydronaphthalene. The monocyclic and the bicyclic cycloalkyl groups can contain one or two alkylene bridges of 1, 2, 3, or 4 carbon atoms, wherein each bridge links two non-adjacent atoms within the groups. Examples of such bridged cycloalkyls include, but are not limited to, bicyclo[2.2.1]heptane, bicyclo[3.1.1]heptane, bicyclo[2.2.2]octane, bicyclo[3.3.1]nonane, adamantane (tricyclo[3.3.1.1^{3,7}]decane), and noradamantane (octahydro-2,5-methanopentalene). Spirocyclic cycloalkyl is exemplified by a monocyclic or a bicyclic cycloalkyl, wherein two of the substituents on the same carbon atom of the ring, together with the carbon atom, form a 4-, 5-, or 6-membered monocyclic cycloalkyl. An example of a spirocyclic cycloalkyl is spiro[2.5]octane. The monocyclic, bicyclic, and spirocyclic cycloalkyl groups are appended to the parent molecular moiety through any substitutable carbon atoms of the groups.

The term “cycloalkylalkyl” as used herein, means a cycloalkyl group, as defined herein, appended to the parent molecular moiety through an alkylene or alkylenyl group, as defined herein.

The term “halo” or “halogen” as used herein, means —Cl, —Br, —I, or —F.

The term “haloalkyl” as used herein, means an alkyl group, as defined herein, in which one, two, three, four, five, six, or seven hydrogen atoms are replaced by halogen. Representative examples of haloalkyl include, but are not limited to, chloromethyl, difluoromethyl, 2-fluoroethyl, 2,2-difluoroethyl, trifluoromethyl, 2,2,2-trifluoroethyl, 2,2,2-trifluoro-1,1-dimethylethyl, difluoromethyl, 3,3,3-trifluoropropyl, pentafluoroethyl, 2-chloro-3-fluoropentyl, and 2-iodoethyl.

The term “heteroaryl,” as used herein, means a monocyclic heteroaryl or a bicyclic heteroaryl. The monocyclic heteroaryl is a 5-or 6-membered ring containing at least one heteroatom independently selected from the group consisting of O, N, and S, where the nitrogen and sulfur heteroatoms can optionally be oxidized and the nitrogen atoms can optionally be quarternized. The 5-membered ring contains two double bonds and one, two, three, or four heteroatoms. The 6-membered ring contains three double bonds and one, two, three, or four heteroatoms. Non limiting examples of monocyclic heteroaryl include furanyl (including furan-2-yl, furan-3-yl), imidazolyl (including 1H-imidazol-1-yl), isoxazolyl, isothiazolyl, oxadiazolyl (including 1,2,4-oxadiazol-5-yl), oxazolyl (including 1,3-oxazol-2-yl), pyridinyl (including pyridin-2-yl, pyridin-4-yl, pyridin-3-yl), pyridazinyl, pyrimidinyl, pyrazinyl, pyrazolyl, pyrrolyl, tetrazolyl, thiadiazolyl, thiazolyl, thienyl (including thien-2-yl, thien-3-yl), triazolyl, and triazinyl. The bicyclic heteroaryl is exemplified by a monocyclic heteroaryl fused to phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Non limiting examples of bicyclic heteroaryls include benzofuranyl, benzoxadiazolyl, 1,3-benzothiazolyl, benzimidazolyl, benzodioxolyl, benzothienyl, 1H-pyrrolo[2,3-b]pyridinyl (including 1H-pyrrolo[2,3-b]pyridin-4-yl), chromenyl, cinnolinyl, furopyridine, indolyl (including 1H-indol-3-yl), indazolyl, isoindolyl, isoquinolinyl, naphthyridinyl, oxazolopyridine, quinolinyl, thienopyridine and thienopyridinyl. The monocyclic and the bicyclic heteroaryl groups are connected to the parent molecular moiety through any substitutable carbon atom or any substitutable nitrogen atom contained within the groups. The nitrogen heteroatoms of the heteroaryl rings can optionally be oxidized, and are contemplated within the scope of the invention.

The term “heteroarylalkyl” as used herein, means a heteroaryl group, as defined herein, appended to the parent molecular moiety through an alkylene or an alkylenyl group, as defined herein.

The term “heterocycle” or “heterocyclic” as used herein, means a monocyclic, bicyclic, or a spirocyclic ring system containing at least one heteroatom selected from nitrogen atom, oxygen atom, and/or sulfur atoms, where the nitrogen and sulfur heteroatoms can optionally be oxidized and the nitrogen atoms can optionally be quarternized. The monocyclic heterocycle is a 3-, 4-5-, 6-, 7-, or 8-membered monocyclic ring containing at least one heteroatom independently selected from the group consisting of O, N, and S. The 3- or 4-membered ring contains 1 heteroatom selected from the group consisting of O, N and S, and optionally one double bond. The 5-membered ring contains zero or one double bond, and one, two or three heteroatoms in the ring selected from the group consisting of O, N and S. The 6-, 7-, or 8-membered ring contains zero, one, or two double bonds, and one, two, or three heteroatoms in the ring selected from the group consisting of O, N and S. Examples of monocyclic heterocycles include, but are not limited to, azetidinyl, azepanyl, aziridinyl, diazepanyl, 1,3-dioxanyl, 1,4-dioxanyl, 1,3-dioxolanyl, 4,5-dihydroisoxazol-5-yl, 3,4-dihydropyran-6-yl, 1,3-dithiolanyl, 1,3-dithianyl, imidazolinyl, imidazolidinyl, isothiazolinyl, isothiazolidinyl, isoxazolinyl, isoxazolidinyl, morpholinyl (including morpholin-4-yl), oxadiazolinyl, oxadiazolidinyl, oxazolinyl, oxazolidinyl, oxetanyl, piperazinyl (including piperazin-1-yl), piperidinyl, pyranyl, pyrazolinyl, pyrazolidinyl, pyrrolinyl, pyrrolidinyl, tetrahydrofuranyl (including tetrahydrofuran-2-yl), tetrahydropyranyl, tetrahydrothienyl (including tetrahydrothien-3-yl), thiadiazolinyl, thiadiazolidinyl, thiazolinyl, thiazolidinyl, thiomorpholinyl, 1,1-dioxidothiomorpholinyl (thiomorpholine sulfone), thiopyranyl, dioxodo-tetrahydrothien-3-yl, and trithianyl. The bicyclic heterocycle is exemplified by a monocyclic heterocycle fused to a phenyl group, or a monocyclic heterocycle fused to a monocyclic cycloalkyl group, or a monocyclic heterocycle fused to a monocyclic cycloalkenyl group, or a monocyclic heterocycle fused to a monocyclic heterocycle group. Examples of bicyclic heterocycle include, but are not limited to, 1,3-benzodioxol-4-yl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, and 1,2,3,4-tetrahydroquinolinyl. Spirocyclic heterocycle means a monocyclic or bicyclic heterocycle ring wherein two substituents on the same carbon atom, together with the carbon atom, form a 4-, 5-, or 6-membered monocyclic cycloalkyl. One example of a spiroheterocycle is 5-oxaspiro[3,4]octane. The heterocycle groups are connected to the parent molecular moiety through any substitutable carbon atom or any substitutable nitrogen atom contained within the group. The monocyclic or bicyclic heterocycle groups of the present invention can contain an alkenylene bridge of 2, 3, or 4 carbon atoms, or one or two alkylene bridges of 1, 2, 3, or 4 carbon atoms, wherein each bridge links two non-adjacent carbon atoms within the groups. Examples of such bridged heterocycles include, but are not limited to, oxaadamantane (2-oxatricyclo[3.3.1.1^{3,7}]decane), octahydro-2,5-epoxypentalene, hexahydro-2H-2,5-methanocyclopenta[b]furan, hexahydro-1H-1,4-methanocyclopenta[c]furan, oxabicyclo[2.2.1]heptane and 2,4-dioxabicyclo[4.2.1]nonane. The nitrogen and sulfur heteroatoms in the heterocycle rings can optionally be oxidized (e.g. 1,1-dioxidotetrahydrothienyl) and the nitrogen atoms can optionally be quarternized.

The term “heterocyclealkyl” as used herein, means a heterocycle group, as defined herein, appended to the parent molecular moiety through an alkylene or an alkylenyl group, as defined herein.

The term “hydroxyalkyl” as used herein, means at least one OH group is appended to the parent molecular moiety through an alkylene or an alkylenyl group, as defined herein. Representative examples of hydroxyalkyl include, but are not limited to, hydroxymethyl, 2-hydroxyethyl, 3-hydroxypropyl, 2,3-dihydroxypropyl, 2,3-dihydroxypentyl, and 2-ethyl-4-hydroxyheptyl.

The term “oxo” means ═O.

The terms “treat”, “treating” and “treatment” refer to a method of alleviating or abrogating a disease and/or its attendant symptoms.

The symbol

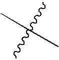

means the point of attachment to the parent moiety.

### b. Compounds

Present compounds have formula (I) as described above.

Particular values of variable groups in compounds of formula (I) are as follows. Such values can be used where appropriate with any of the other values, definitions, claims or embodiments defined hereinbefore or hereinafter.

As described generally in the Summary section for compounds of formula (I), A is formula (i), (ii), or (iii).

Certain embodiments are directed to a group of compounds of formula (I) wherein A is formula (i). Thus, compounds within formula (I) include compounds of formula (Ia) and pharmaceutically acceptable salts or solvates thereof:

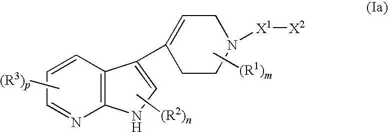

Other embodiments are directed to a group of compounds of formula (I) wherein A is formula (ii). Thus, compounds within formula (I) include compounds of formula (Ib) and pharmaceutically acceptable salts or solvates thereof:

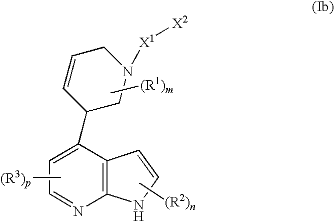

Yet another group of compounds formula (I) include those in which A is formula (iii). Thus the provided herein is a group of compounds of formula (Ic) and pharmaceutically acceptable salts or solvates thereof:

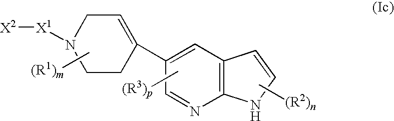

For each substructure as defined by ring A, there exist the following embodiments which further define the scope of the compounds. These further embodiments are contemplated to apply to each series of compounds of formula (I), (Ia), (Ib) and (Ic).

As described generally above for compounds of formula (I), (Ia), (Ib), or (Ic), X¹ and X² have values as disclosed in the Summary.

For example, one aspect is directed to any of the group of compounds of formula (I), (Ia), (Ib), and (Ic) wherein X¹ is C(O), C(O)N(R⁴), C(O)O, or S(O)₂, X² is —(CR^{6a}R^{6b})_{q}-G¹, —(CR^{6a}R^{6b})ᵣ—X³-G¹, or J^{A}, and X³, R⁴, R^{6a}, R^{6b}, G¹, J^{A}, r, and q are as described generally in the Summary and in the embodiments herein.

Another aspect is directed to any of the group of compounds of formula (I), (Ia), (Ib), and (Ic) wherein X¹ is C(O), X² is —(CR^{6a}R^{6b})_{q}-G¹ or J^{A}, and R^{6a}, R^{6b}, G¹, J^{A}, and q are as described generally in the Summary and in the embodiments herein.

Another aspect is directed to any of the group of compounds of formula (I), (Ia), (Ib), and (Ic) wherein X¹ is C(O)N(R⁴), X² is —(CR^{6a}R^{6b})_{q}-G¹, —(CR^{6a}R^{6b})ᵣ—X³-G¹, or J^{A}, and X³, R⁴, R^{6a}, R^{6b}, G¹, J^{A}, r, and q are as described generally in the Summary and in the embodiments herein.

Another aspect is directed to any of the group of compounds of formula (I), (Ia), (Ib), and (Ic) wherein X¹ is C(O)N(R⁴), X² is —(CR^{6a}R^{6b})_{q}-G¹, and R⁴, R^{6a}, R^{6b}, G¹, and q are as described generally in the Summary and in the embodiments herein.

Another aspect is directed to any of the group of compounds of formula (I), (Ia), (Ib), and (Ic) wherein X¹ is C(O)N(R⁴), X² is —(CR^{6a}R^{6b})ᵣ—X³-G¹, and X³, R⁴, R^{6a}, R^{6b}, G¹, and r are as described generally in the Summary and in the embodiments herein. For example, X³ is O. In certain embodiments, r is 2.

Another aspect is directed to any of the group of compounds of formula (I), (Ia), (Ib), and (Ic) wherein X¹ is C(O)N(R⁴), X² is J^{A}, and R⁴ and J^{A} are as described generally in the Summary and in the embodiments herein.

Yet another aspect is directed to any of the group of compounds of formula (I), (Ia), (Ib), and (Ic) wherein X¹ is —C(O)O, X² is —(CR^{6a}R^{6b})_{q}-G¹, and R^{6a}, R^{6b}, G¹, and q are as described generally in the Summary and in the embodiments herein.

A further aspect is directed to any of the group of compounds of formula (I), (Ia), (Ib), and (Ic) wherein X¹ is S(O)₂, X² is —(CR^{6a}R^{6b})_{q}-G¹, and R^{6a}, R^{6b}, G¹, and q are as described generally in the Summary and in the embodiments herein.

Still another aspect is directed to any of the group of compounds of formula (I), (Ia), (Ib), and (Ic) wherein X¹-X² together is a five membered monocyclic heterocycle or a five membered monocyclic heteroaryl ring, each of which is optionally substituted as described in the Summary and embodiments herein. In certain embodiments, X¹-X² together is an optionally substituted five-membered monocyclic heterocycle (e.g. optionally substituted dihydro-1,3-oxazolyl). In yet other embodiments X¹-X² together is an optionally substituted five-membered monocyclic heteroaryl ring (for example, 1,2,4-oxadiazolyl or oxazolyl, each of which is optionally substituted). In certain embodiments, the five membered monocyclic heterocycle or five membered monocyclic heteroaryl ring aryl is substituted with one G² such as, but not limited to, aryl (for example, phenyl) and heteroaryl (for example, pyridinyl), each of which is independently further substituted as described in the Summary; and optionally further substituted with one other R^{7c} group such as, for example, alkyl, halogen, or haloalkyl. In certain embodiments, X¹-X² together is (iv), (v), or (vi)

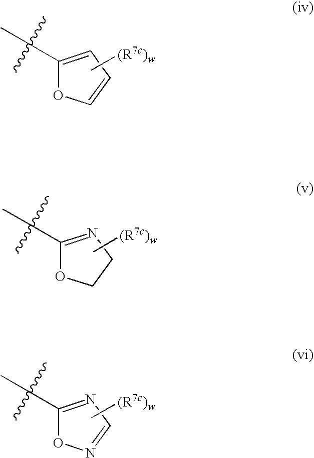

wherein R^{7c} is as disclosed in the Summary and in embodiments herein above, and w is 1 or 2.

R⁴, for example, includes hydrogen and alkyl (e.g. methyl). In certain embodiments, R⁴ is hydrogen.

Within each group of compounds as described herein, R^{6a} and R^{6b} have values as described in the Summary and in embodiments herein. For example, R^{6a} and R^{6b}, at each occurrence, are each independently hydrogen, alkyl (e.g. methyl), optionally substituted aryl (e.g. optionally substituted phenyl), arylalkyl (such as, but not limited to, benzyl), or alkyl substituted with one —OR^{u} group wherein R^{u} is as described in the Summary and embodiments herein. For example, R^{u} is hydrogen. In certain embodiments, R^{6a} and R^{6b}, at each occurrence, are each independently hydrogen, alkyl (e.g. methyl), unsubstituted or substituted phenyl, or —CH₂OH.

Within each group of compounds as described herein, non limiting examples of G¹ include cycloalkyl (e.g. cyclohexyl), cycloalkenyl (e.g. cyclohexenyl), heteroaryl (e.g. thienyl, furanyl, pyridinyl, imidazolyl, oxazolyl, indolyl), heterocycle (e.g. tetrahydrothienyl, tetrahydrofuranyl, dioxidotetrahydrothienyl), and aryl (e.g. phenyl, naphthyl). In certain embodiments, G¹ is aryl (e.g. phenyl, naphthyl). Each G¹ is independently unsubstituted or substituted with 1, 2, 3, 4, 5 substituents as represented by R^{7b}. Examples of R^{7b} include, but are not limited to, alkyl (e.g. methyl), halogen (e.g. Br, F, Cl, I), haloalkyl (e.g. trifluoroalkyl), OR^{7ab}, SR^{7ab}, N(R^{b})(R^{7ab}), C(O)NR^{7ab}R^{b}, and —O(CR^{ax}R^{bx})ₜO—, wherein R^{b}, R^{7ab}, R^{ax}, R^{bx}, and t are as described in the Summary and in the embodiments herein. For example, each occurrence of R^{7ab} is independently hydrogen, alkyl (e.g. methyl, ethyl, propyl), haloalkyl (e.g. trifluoromethyl), or —(C₁₋₆ alkylene)-G² wherein G² is a heterocycle such as, but not limited to, morpholinyl, pyrrolidinyl, piperidinyl, piperazinyl, tetrahydropyranyl, and tetrahydrofuranyl; each of which is optionally substituted as described in the Summary. In certain embodiments, R^{7b} is alkyl (e.g. methyl, ethyl), halogen (e.g. Br, F, Cl, I), haloalkyl (e.g. trifluoroalkyl), OR^{7ab}, SR^{7ab}, or —O(CR^{ax}R^{bx})ₜO—; wherein each occurrence of R^{7ab} is independently hydrogen, alkyl (e.g. methyl, ethyl, propyl), or haloalkyl (e.g. trifluoromethyl).

Within each of the groups of formula (I), (Ia), (Ib), and (Ic), J^{A} has values as described generally in the Summary and in embodiments herein. For example, in certain embodiments, J^{A} is a monocyclic cycloalkyl optionally substituted with 1, 2, 3, 4, 5, or 6 substituents as represented by R^{7JA}; two R^{7JA} on the adjacent carbon atoms of J^{A}, together with the carbon atoms to which they are attached, optionally form a benzo, a monocyclic heterocycle, a monocyclic cycloalkyl, or a monocyclic cycloalkenyl ring wherein each of the rings is independently unsubstituted or substituted with 1, 2, or 3 substituents as represented by R^{7b}. For example, J^{A} is a monocyclic cyclohexyl fused with a benzo group. In certain embodiments, J^{A} is an optionally substituted monocyclic heterocycle ring. Non-limiting examples of the optionally substituted monocyclic heterocycle ring include piperazinyl, pyrrolidinyl, piperidinyl, morpholinyl, each of which is optionally substituted as described in the Summary and embodiments herein. For example, the optional substituents of J^{A} include, but are not limited to, alkyl (e.g. methyl, ethyl, propyl, isopropyl) and G² (e.g. optionally substituted aryl such as, but not limited to, optionally substituted phenyl).

Within any one of the groups of compounds of formula (I), (Ia), (Ib), and (Ic), m, n, and p have values as described generally in the Summary and embodiments herein. In certain embodiments, m, n, and p are 0. In yet other embodiments, m is 1, and n and p are as described in the Summary.

Within any one of the groups of compounds of formula (I), (Ia), (Ib), and (Ic), R¹, R² and R³ are as described generally in the Summary and embodiments herein. In certain embodiments, R¹ is alkyl such as, but not limited to, methyl. R², for example, is aryl (e.g. phenyl) or arylalkyl (e.g. benzyl). R³, for example, is halogen (e.g. Cl, Br) or NR^{a}R^{b}.

Exemplary compounds include, but are not limited to:

N-[(1S)-2-hydroxy-1-phenylethyl]-4-(1H-pyrrolo[2,3-b]pyridin-3-yl)-3,6-dihydropyridine-1(2H)-carboxamide;

3-[1-(3-phenylpropanoyl)-1,2,3,6-tetrahydropyridin-4-yl]-1H-pyrrolo[2,3-b]pyridine;

3-{1-[(2-phenylethyl)sulfonyl]-1,2,3,6-tetrahydropyridin-4-yl}-1H-pyrrolo[2,3-b]pyridine;

N-benzyl-4-(1H-pyrrolo[2,3-b]pyridin-3-yl)-3,6-dihydropyridine-1(2H)-carboxamide;

N-(1-naphthylmethyl)-4-(1H-pyrrolo[2,3-b]pyridin-3-yl)-3,6-dihydropyridine-1(2H)-carboxamide;

3-{1-[(3-phenylmorpholin-4-yl)carbonyl]-1,2,3,6-tetrahydropyridin-4-yl}-1H-pyrrolo[2,3-b]pyridine;

3-{1-[(4-methyl-2-phenylpiperazin-1-yl)carbonyl]-1,2,3,6-tetrahydropyridin-4-yl}-1H-pyrrolo[2,3-b]pyridine;

N-[(1S)-1-phenylethyl]-4-(1H-pyrrolo[2,3-b]pyridin-3-yl)-3,6-dihydropyridine-1(2H)-carboxamide;

N-[(1R)-1-phenylethyl]-4-(1H-pyrrolo[2,3-b]pyridin-3-yl)-3,6-dihydropyridine-1(2H)-carboxamide;

N-(2-phenoxyethyl)-4-(1H-pyrrolo[2,3-b]pyridin-3-yl)-3,6-dihydropyridine-1(2H)-carboxamide;

N-(2-phenylethyl)-4-(1H-pyrrolo[2,3-b]pyridin-3-yl)-3,6-dihydropyridine-1(2H)-carboxamide;

N-(2,4-dichlorobenzyl)-4-(1H-pyrrolo[2,3-b]pyridin-3-yl)-3,6-dihydropyridine-1(2H)-carboxamide;

N-(2-chlorobenzyl)-4-(1H-pyrrolo[2,3-b]pyridin-3-yl)-3,6-dihydropyridine-1(2H)-carboxamide;

N-(3,4-dichlorobenzyl)-4-(1H-pyrrolo[2,3-b]pyridin-3-yl)-3,6-dihydropyridine-1(2H)-carboxamide;

N-(4-fluorobenzyl)-4-(1H-pyrrolo[2,3-b]pyridin-3-yl)-3,6-dihydropyridine-1(2H)-carboxamide;

N-(4-methoxybenzyl)-4-(1H-pyrrolo[2,3-b]pyridin-3-yl)-3,6-dihydropyridine-1(2H)-carboxamide;

N-(3-methylbenzyl)-4-(1H-pyrrolo[2,3-b]pyridin-3-yl)-3,6-dihydropyridine-1(2H)-carboxamide;

N-(4-methylbenzyl)-4-(1H-pyrrolo[2,3-b]pyridin-3-yl)-3,6-dihydropyridine-1(2H)-carboxamide;

N-(2-methylbenzyl)-4-(1H-pyrrolo[2,3-b]pyridin-3-yl)-3,6-dihydropyridine-1(2H)-carboxamide;

N-(4-bromobenzyl)-4-(1H-pyrrolo[2,3-b]pyridin-3-yl)-3,6-dihydropyridine-1(2H)-carboxamide;

N-(2-fluorobenzyl)-4-(1H-pyrrolo[2,3-b]pyridin-3-yl)-3,6-dihydropyridine-1(2H)-carboxamide;

N-(3-fluorobenzyl)-4-(1H-pyrrolo[2,3-b]pyridin-3-yl)-3,6-dihydropyridine-1(2H)-carboxamide;

4-(1H-pyrrolo[2,3-b]pyridin-3-yl)-N-(3,4,5-trimethoxybenzyl)-3,6-dihydropyridine-1(2H)-carboxamide;

N-(2-methoxybenzyl)-4-(1H-pyrrolo[2,3-b]pyridin-3-yl)-3,6-dihydropyridine-1(2H)-carboxamide;

N-(2-ethoxybenzyl)-4-(1H-pyrrolo[2,3-b]pyridin-3-yl)-3,6-dihydropyridine-1(2H)-carboxamide;

N-(3-methoxybenzyl)-4-(1H-pyrrolo[2,3-b]pyridin-3-yl)-3,6-dihydropyridine-1(2H)-carboxamide;

N-[2-(1,3-benzodioxol-5-yl)ethyl]-4-(1H-pyrrolo[2,3-b]pyridin-3-yl)-3,6-dihydropyridine-1(2H)-carboxamide;

N-[2-(3,5-dimethoxyphenyl)ethyl]-4-(1H-pyrrolo[2,3-b]pyridin-3-yl)-3,6-dihydropyridine-1(2H)-carboxamide;

N-[2-(2,3-dimethoxyphenyl)ethyl]-4-(1H-pyrrolo[2,3-b]pyridin-3-yl)-3,6-dihydropyridine-1(2H)-carboxamide;

N-[2-(3,4-dichlorophenyl)ethyl]-4-(1H-pyrrolo[2,3-b]pyridin-3-yl)-3,6-dihydropyridine-1(2H)-carboxamide;

N-[2-(2,6-dichlorophenyl)ethyl]-4-(1H-pyrrolo[2,3-b]pyridin-3-yl)-3,6-dihydropyridine-1(2H)-carboxamide;

N-[2-(5-bromo-2-methoxyphenyl)ethyl]-4-(1H-pyrrolo[2,3-b]pyridin-3-yl)-3,6-dihydropyridine-1(2H)-carboxamide;

N-[2-(3-bromo-4-methoxyphenyl)ethyl]-4-(1H-pyrrolo[2,3-b]pyridin-3-yl)-3,6-dihydropyridine-1(2H)-carboxamide;

N-[2-(2,5-dimethoxyphenyl)ethyl]-4-(1H-pyrrolo[2,3-b]pyridin-3-yl)-3,6-dihydropyridine-1(2H)-carboxamide;

N-(4-chlorobenzyl)-4-(1H-pyrrolo[2,3-b]pyridin-3-yl)-3,6-dihydropyridine-1(2H)-carboxamide;

N-[2-(2-fluorophenyl)ethyl]-4-(1H-pyrrolo[2,3-b]pyridin-3-yl)-3,6-dihydropyridine-1(2H)-carboxamide;

N-[2-(4-methoxyphenyl)ethyl]-4-(1H-pyrrolo[2,3-b]pyridin-3-yl)-3,6-dihydropyridine-1(2H)-carboxamide;

N-[2-(3-chlorophenyl)ethyl]-4-(1H-pyrrolo[2,3-b]pyridin-3-yl)-3,6-dihydropyridine-1(2H)-carboxamide;

N-[2-(2,4-dichlorophenyl)ethyl]-4-(1H-pyrrolo[2,3-b]pyridin-3-yl)-3,6-dihydropyridine-1(2H)-carboxamide;

N-[2-(4-fluorophenyl)ethyl]-4-(1H-pyrrolo[2,3-b]pyridin-3-yl)-3,6-dihydropyridine-1(2H)-carboxamide;

N-(2,2-diphenylethyl)-4-(1H-pyrrolo[2,3-b]pyridin-3-yl)-3,6-dihydropyridine-1(2H)-carboxamide;

N-[2-(3,4-dimethoxyphenyl)ethyl]-4-(1H-pyrrolo[2,3-b]pyridin-3-yl)-3,6-dihydropyridine-1(2H)-carboxamide;

N-[2-(4-chlorophenyl)ethyl]-4-(1H-pyrrolo[2,3-b]pyridin-3-yl)-3,6-dihydropyridine-1(2H)-carboxamide;

N-(cyclohexylmethyl)-4-(1H-pyrrolo[2,3-b]pyridin-3-yl)-3,6-dihydropyridine-1(2H)-carboxamide;

N-(4-phenylbutyl)-4-(1H-pyrrolo[2,3-b]pyridin-3-yl)-3,6-dihydropyridine-1(2H)-carboxamide;

N-[(1,1-dioxidotetrahydrothien-3-yl)methyl]-4-(1H-pyrrolo[2,3-b]pyridin-3-yl)-3,6-dihydropyridine-1(2H)-carboxamide;

4-(1H-pyrrolo[2,3-b]pyridin-3-yl)-N-(2-thien-2-ylethyl)-3,6-dihydropyridine-1(2H)-carboxamide;

N-(2-furylmethyl)-4-(1H-pyrrolo[2,3-b]pyridin-3-yl)-3,6-dihydropyridine-1(2H)-carboxamide;

N-(3-phenylpropyl)-4-(1H-pyrrolo[2,3-b]pyridin-3-yl)-3,6-dihydropyridine-1(2H)-carboxamide;

N-(pyridin-3-ylmethyl)-4-(1H-pyrrolo[2,3-b]pyridin-3-yl)-3,6-dihydropyridine-1(2H)-carboxamide;

3-(1-{4-methyl-5-[3-(trifluoromethyl)phenyl]-1,3-oxazol-2-yl}-1,2,3,6-tetrahydropyridin-4-yl)-1H-pyrrolo[2,3-b]pyridine;

N-(2,3-dihydro-1,4-benzodioxin-5-ylmethyl)-4-(1H-pyrrolo[2,3-b]pyridin-3-yl)-3,6-dihydropyridine-1(2H)-carboxamide;

N-methyl-N-[(1R)-1-phenylethyl]-4-(1H-pyrrolo[2,3-b]pyridin-3-yl)-3,6-dihydropyridine-1(2H)-carboxamide;

benzyl 4-(1H-pyrrolo[2,3-b]pyridin-3-yl)-3,6-dihydropyridine-1(2H)-carboxylate;

2-chlorobenzyl 4-(1H-pyrrolo[2,3-b]pyridin-3-yl)-3,6-dihydropyridine-1(2H)-carboxylate;

N-[1-(2-chlorophenyl)ethyl]-4-(1H-pyrrolo[2,3-b]pyridin-3-yl)-3,6-dihydropyridine-1(2H)-carboxamide;

3-{1-[(4S)-4-phenyl-4,5-dihydro-1,3-oxazol-2-yl]-1,2,3,6-tetrahydropyridin-4-yl}-1H-pyrrolo[2,3-b]pyridine;

N-[3-fluoro-5-(trifluoromethyl)benzyl]-4-(1H-pyrrolo[2,3-b]pyridin-3-yl)-3,6-dihydropyridine-1(2H)-carboxamide;

4-(1H-pyrrolo[2,3-b]pyridin-3-yl)-N-{4-[(trifluoromethyl)thio]benzyl}-3,6-dihydropyridine-1(2H)-carboxamide;

4-(1H-pyrrolo[2,3-b]pyridin-3-yl)-N-[4-(trifluoromethoxy)benzyl]-3,6-dihydropyridine-1(2H)-carboxamide;

4-(1H-pyrrolo[2,3-b]pyridin-3-yl)-N-[3-(trifluoromethoxy)benzyl]-3,6-dihydropyridine-1(2H)-carboxamide;

N-(2,3-dimethoxybenzyl)-4-(1H-pyrrolo[2,3-b]pyridin-3-yl)-3,6-dihydropyridine-1(2H)-carboxamide;

N-(2,5-difluorobenzyl)-4-(1H-pyrrolo[2,3-b]pyridin-3-yl)-3,6-dihydropyridine-1(2H)-carboxamide;

4-(1H-pyrrolo[2,3-b]pyridin-3-yl)-N-1,2,3,4-tetrahydronaphthalen-1-yl-3,6-dihydropyridine-1(2H)-carboxamide;

N-(2,6-difluorobenzyl)-4-(1H-pyrrolo[2,3-b]pyridin-3-yl)-3,6-dihydropyridine-1(2H)-carboxamide;

N-(1,2-diphenylethyl)-4-(1H-pyrrolo[2,3-b]pyridin-3-yl)-3,6-dihydropyridine-1(2H)-carboxamide;

N-(2,4-difluorobenzyl)-4-(1H-pyrrolo[2,3-b]pyridin-3-yl)-3,6-dihydropyridine-1(2H)-carboxamide;

N-(2,5-dimethoxybenzyl)-4-(1H-pyrrolo[2,3-b]pyridin-3-yl)-3,6-dihydropyridine-1(2H)-carboxamide;

N-(2,3-dichlorobenzyl)-4-(1H-pyrrolo[2,3-b]pyridin-3-yl)-3,6-dihydropyridine-1(2H)-carboxamide;

N-(3,5-dichlorobenzyl)-4-(1H-pyrrolo[2,3-b]pyridin-3-yl)-3,6-dihydropyridine-1(2H)-carboxamide;

N-(2-cyclohex-1-en-1-ylethyl)-4-(1H-pyrrolo[2,3-b]pyridin-3-yl)-3,6-dihydropyridine-1(2H)-carboxamide;

N-(3,3-diphenylpropyl)-4-(1H-pyrrolo[2,3-b]pyridin-3-yl)-3,6-dihydropyridine-1(2H)-carboxamide;

N-[2-(1H-indol-3-yl)ethyl]-4-(1H-pyrrolo[2,3-b]pyridin-3-yl)-3,6-dihydropyridine-1(2H)-carboxamide;

4-(1H-pyrrolo[2,3-b]pyridin-3-yl)-N-(thien-2-ylmethyl)-3,6-dihydropyridine-1(2H)-carboxamide;

3-[1-(3-pyridin-3-yl-1,2,4-oxadiazol-5-yl)-1,2,3,6-tetrahydropyridin-4-yl]-1H-pyrrolo[2,3-b]pyridine;

N-[(1R)-1-(3-methoxyphenyl)ethyl]-4-(1H-pyrrolo[2,3-b]pyridin-3-yl)-3,6-dihydropyridine-1(2H)-carboxamide;

N-[(1R)-1-(3-methoxyphenyl)ethyl]-4-(1H-pyrrolo[2,3-b]pyridin-4-yl)-3,6-dihydropyridine-1(2H)-carboxamide; and

N-[(1R)-1-(3-methoxyphenyl)ethyl]-4-(1H-pyrrolo[2,3-b]pyridin-5-yl)-3,6-dihydropyridine-1(2H)-carboxamide.

Other compounds or pharmaceutically acceptable salts or solvates thereof that are contemplated include, but are not limited to,

N-(2,5-dichlorobenzyl)-4-(1H-pyrrolo[2,3-b]pyridin-3-yl)-3,6-dihydropyridine-1(2H)-carboxamide;

N-[(5-methyl-2-furyl)methyl]-4-(1H-pyrrolo[2,3-b]pyridin-3-yl)-3,6-dihydropyridine-1(2H)-carboxamide;

N-(3-iodobenzyl)-4-(1H-pyrrolo[2,3-b]pyridin-3-yl)-3,6-dihydropyridine-1(2H)-carboxamide;

N-[(4-chlorophenyl)(phenyl)methyl]-4-(1H-pyrrolo[2,3-b]pyridin-3-yl)-3,6-dihydropyridine-1(2H)-carboxamide;

N-[3,5-bis(trifluoromethyl)benzyl]-4-(1H-pyrrolo[2,3-b]pyridin-3-yl)-3,6-dihydropyridine-1(2H)-carboxamide;

N-[3-(1H-imidazol-1-yl)propyl]-4-(1H-pyrrolo[2,3-b]pyridin-3-yl)-3,6-dihydropyridine-1(2H)-carboxamide;

N-(2-bromobenzyl)-4-(1H-pyrrolo[2,3-b]pyridin-3-yl)-3,6-dihydropyridine-1(2H)-carboxamide;

4-(1H-pyrrolo[2,3-b]pyridin-3-yl)-N-[3-(trifluoromethyl)benzyl]-3,6-dihydropyridine-1(2H)-carboxamide;

4-(1H-pyrrolo[2,3-b]pyridin-3-yl)-N-[(2S)-tetrahydrofuran-2-ylmethyl]-3,6-dihydropyridine-1(2H)-carboxamide;

N-(2,3-dihydroxypropyl)-4-(1H-pyrrolo[2,3-b]pyridin-3-yl)-3,6-dihydropyridine-1(2H)-carboxamide;

4-(1H-pyrrolo[2,3-b]pyridin-3-yl)-N-(thien-3-ylmethyl)-3,6-dihydropyridine-1(2H)-carboxamide;

N-[(1R)-1-(3-methoxyphenyl)ethyl]-4-(1H-pyrrolo[2,3-b]pyridin-3-yl)-3,6-dihydropyridine-1(2H)-carboxamide;

N-[3-(2-morpholin-4-ylethoxy)benzyl]-4-(1H-pyrrolo[2,3-b]pyridin-3-yl)-3,6-dihydropyridine-1(2H)-carboxamide;

N-[3-(2-morpholin-4-ylethoxy)benzyl]-4-(1H-pyrrolo[2,3-b]pyridin-4-yl)-3,6-dihydropyridine-1(2H)-carboxamide;

N-[3-(2-morpholin-4-ylethoxy)benzyl]-4-(1H-pyrrolo[2,3-b]pyridin-5-yl)-3,6-dihydropyridine-1(2H)-carboxamide;

4-(1H-pyrrolo[2,3-b]pyridin-3-yl)-N-[3-(2-tetrahydrofuran-2-ylethoxy)benzyl]-3,6-dihydropyridine-1(2H)-carboxamide;

4-(1H-pyrrolo[2,3-b]pyridin-4-yl)-N-[3-(2-tetrahydrofuran-2-ylethoxy)benzyl]-3,6-dihydropyridine-1(2H)-carboxamide;

4-(1H-pyrrolo[2,3-b]pyridin-5-yl)-N-[3-(2-tetrahydrofuran-2-ylethoxy)benzyl]-3,6-dihydropyridine-1(2H)-carboxamide;

N-(1-phenyl-3-tetrahydrofuran-2-ylpropyl)-4-(1H-pyrrolo[2,3-b]pyridin-3-yl)-3,6-dihydropyridine-1(2H)-carboxamide;

N-(1-phenyl-3-tetrahydrofuran-2-ylpropyl)-4-(1H-pyrrolo[2,3-b]pyridin-4-yl)-3,6-dihydropyridine-1(2H)-carboxamide;

N-(1-phenyl-3-tetrahydrofuran-2-ylpropyl)-4-(1H-pyrrolo[2,3-b]pyridin-5-yl)-3,6-dihydropyridine-1(2H)-carboxamide;

N-(1-phenyl-3-pyrrolidin-1-ylpropyl)-4-(1H-pyrrolo[2,3-b]pyridin-3-yl)-3,6-dihydropyridine-1(2H)-carboxamide;

N-(1-phenyl-3-pyrrolidin-1-ylpropyl)-4-(1H-pyrrolo[2,3-b]pyridin-4-yl)-3,6-dihydropyridine-1(2H)-carboxamide;

N-(1-phenyl-3-pyrrolidin-1-ylpropyl)-4-(1H-pyrrolo[2,3-b]pyridin-5-yl)-3,6-dihydropyridine-1(2H)-carboxamide;

N-(4-fluoro-3-methoxybenzyl)-4-(1H-pyrrolo[2,3-b]pyridin-3-yl)-3,6-dihydropyridine-1(2H)-carboxamide;

N-(4-fluoro-3-methoxybenzyl)-4-(1H-pyrrolo[2,3-b]pyridin-4-yl)-3,6-dihydropyridine-1(2H)-carboxamide;

N-(4-fluoro-3-methoxybenzyl)-4-(1H-pyrrolo[2,3-b]pyridin-5-yl)-3,6-dihydropyridine-1(2H)-carboxamide;

N-(3-propoxybenzyl)-4-(1H-pyrrolo[2,3-b]pyridin-3-yl)-3,6-dihydropyridine-1(2H)-carboxamide;

N-(3-propoxybenzyl)-4-(1H-pyrrolo[2,3-b]pyridin-4-yl)-3,6-dihydropyridine-1(2H)-carboxamide;

N-(3-propoxybenzyl)-4-(1H-pyrrolo[2,3-b]pyridin-5-yl)-3,6-dihydropyridine-1(2H)-carboxamide;

N-(3-{[(2-morpholin-4-ylethyl)amino]carbonyl}benzyl)-4-(1H-pyrrolo[2,3-b]pyridin-3-yl)-3,6-dihydropyridine-1(2H)-carboxamide;

N-(3-{[(2-morpholin-4-ylethyl)amino]carbonyl}benzyl)-4-(1H-pyrrolo[2,3-b]pyridin-4-yl)-3,6-dihydropyridine-1(2H)-carboxamide; and

N-(3-{[(2-morpholin-4-ylethyl)amino]carbonyl}benzyl)-4-(1H-pyrrolo[2,3-b]pyridin-5-yl)-3,6-dihydropyridine-1(2H)-carboxamide.

As described herein, a bond drawn from a substituent to the center of one ring within a bicyclic ring system as shown in formula (Ia), (Ib), (Ic), (i), (ii), and (iii), represents substitution of the substituents at any substitutable carbon atoms within the bicyclic ring system, unless stated otherwise.

It is appreciated that certain compounds described herein can exist as stereoisomers wherein at least one asymmetric or chiral center is present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30.

Individual stereoisomers (including enantiomers and diastereomers), as well as the mixtures of various ratio of the enantiomers and diastereomers of the compounds (including racemates), are contemplated in the present application. Individual stereoisomers can be prepared synthetically from commercially available chiral reagents or by stereoselective or stereospecific synthetic techniques. Alternatively, the single enantiomers or diastereomers can be obtained from the preparation of racemic mixtures followed by resolution of the individual stereoisomer using methods that are known to those of ordinary skill in the art. Examples of resolution are, for example, (i) attachment of a mixture of enantiomers to a chiral auxiliary, separation of the resulting mixture of diastereomers by recrystallization or chromatography, followed by liberation of the optically pure product; or (ii) separation of the mixture of enantiomers or diastereomers on chiral chromatographic columns.

Geometric isomers can also exist in the present compounds. Various geometric isomers and mixtures thereof resulting from the disposition of substituents around a carbon-carbon double bond, a carbon-nitrogen double bond, a cycloalkyl group, or a heterocycle group are also contemplated. Substituents around a carbon-carbon double bond or a carbon-nitrogen bond are designated as being of Z or E configuration and substituents around a cycloalkyl or a heterocycle are designated as being of cis or trans configuration. The individual geometric isomers can be prepared selectively by methods known to the skilled artisan, or mixtures of the isomers can be separated by standard chromatographic or crystallization techniques.

It is to be understood that compounds disclosed herein can exhibit the phenomenon of tautomerism. All tautomeric forms and mixtures thereof are contemplated.

Thus, the formulae drawings within this specification can represent only one of the possible tautomeric or stereoisomeric forms. It is to be understood that any tautomeric or stereoisomeric form, and mixtures thereof are encompassed, and is not to be limited merely to any one tautomeric or stereoisomeric form utilized within the naming of the compounds or formulae drawings.

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to, ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

In another embodiment, the isotope-labeled compounds contain deuterium (²H), tritium (³H) or ¹⁴C isotopes. Isotope-labeled compounds of this invention can be prepared by the general methods well known to persons having ordinary skill in the art. Such isotope-labeled compounds can be conveniently prepared by carrying out the procedures disclosed in the Examples disclosed herein and Schemes by substituting a readily available isotope-labeled reagent for a non-labeled reagent. In some instances, compounds can be treated with isotope-labeled reagents to exchange a normal atom with its isotope, for example, hydrogen for deuterium can be exchanged by the action of a deuteric acid such as D₂SO₄/D₂O. In addition to the above, relevant procedures and intermediates are disclosed, for instance, in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; and 20090082471, the methods are hereby incorporated by reference.

The isotope-labeled compounds of the invention can be used as standards to determine the effectiveness of ROCK inhibitors in binding assays. Isotope containing compounds have been used in pharmaceutical research to investigate the in vivo metabolic fate of the compounds by evaluation of the mechanism of action and metabolic pathway of the nonisotope-labeled parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

In addition, non-radio active isotope containing drugs, such as deuterated drugs called “heavy drugs,” can be used for the treatment of diseases and conditions related to inhibition of ROCK. Increasing the amount of an isotope present in a compound above its natural abundance is called enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Replacement of up to about 15% of normal atom with a heavy isotope has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci 1960 84: 736; Czakja D M et al., Am. J. Physiol. 1961 201: 357). Acute replacement of as high as 15%-23% in human fluids with deuterium was found not to cause toxicity (Blagojevic N et al. in “Dosimetry & Treatment Planning for Neutron Capture Therapy”, Zamenhof R, Solares G and Harling O Eds. 1994. Advanced Medical Publishing, Madison Wisc. pp. 125-134; Diabetes Metab. 23: 251 (1997)).

Stable isotope labeling of a drug can alter its physico-chemical properties such as pKa and lipid solubility. These effects and alterations can affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one important exception: because of the increased mass of the heavy isotope, any bond involving the heavy isotope and another atom can be stronger than the same bond between the light isotope and that atom. Accordingly, the incorporation of an isotope at a site of metabolism or enzymatic transformation can slow the reactions potentially altering the pharmcokinetic profile or efficacy relative to the non-isotopic compound.

### c. Biological Data

(i) In Vitro Methods

ROCK-2 Inhibitory Assay

Certain compounds were tested for their ability to inhibit N-terminal His6-tagged recombinant human ROCK-2 residues 11-552 expressed by baculovirus in Sf21 cells (Upstate). In 384-well v-bottom polypropylene plates (Axygen), 1 nM (final concentration) in 10 μL recombinant N-terminal His6-tagged recombinant human ROCK-2 residues 11-552 expressed by baculovirus in Sf21 cells (Upstate) was mixed with 2 μM (final concentration) in 10 μL biotinylated peptide substrate (biotin-Aha-K-E-A-K-E-K-R-Q-E-Q-I-A-K-R-R-R-L-S-S-L-R-A-S-T-S-K-S-G-G-S-Q-K) (Genemed), and various concentration of inhibitor (2% DMSO final) in reaction buffer (25 mM HEPES, pH 7.5, 0.5 mM DTT, 10 mM MgCl₂, 100 uM Na₃VO₄, 0.075 mg/ml Triton X-100), and the reaction was initiated by addition of 5 uM unlabelled ATP containing 0.01 μCi [³³P]-ATP (Perkin Elmer). The reaction was quenched after 1 hour by the addition of 50 μL stop buffer (50 mM EDTA, 2M NaCl final concentration). 80 μL of the stopped reactions were transferred to 384-well streptavidin-coated FlashPlates (Perkin Elmer), incubated 10 minutes at room temperature, washed 3 times with 0.05% Tween-20/PBS using an ELX-405 automated plate washer (BioTek), and counted on a TopCount Scintillation Plate Reader (Packard).

ROCK-1 Inhibitory Assay

Certain compounds were tested for their ability to inhibit N-terminal His6-tagged, recombinant, human ROCK-1 amino acids 17-535 expressed by baculovirus in Sf21 cells (Upstate). In 384-well v-bottom polypropylene plates (Axygen), 2 nM (final concentration) in 10 μL recombinant N-terminal His6-tagged, recombinant, human ROCK-1 amino acids 17-535 expressed by baculovirus in Sf21 cells (Upstate) in reaction buffer was mixed with 2 uM (final concentration) biotinylated peptide substrate (biotin-Aha-V-R-R-L-R-R-L-T-A-R-E-A-A) (Genemed), and various concentration of inhibitor (2% DMSO final) in 10 μL reaction buffer (25 mM HEPES, pH 7.5, 0.5 mM DTT, 10 mM MgCl₂, 100 μM Na₃VO₄, 0.075 mg/ml Triton X-100), and the reaction was initiated by addition of 5 uM unlabelled ATP containing 0.01 μCi [³³P]-ATP (Perkin Elmer). The reaction was quenched after 1 hour by the addition of 50 μL stop buffer (50 mM EDTA, 2M NaCl final concentration). 80 μL of the stopped reactions were transferred to 384-well streptavidin-coated FlashPlates (Perkin Elmer), incubated 10 minutes at room temperature, washed 3 times with 0.05% Tween-20/PBS using an ELX-405 automated plate washer (BioTek), and counted on a TopCount Scintillation Plate Reader (Packard).

Compounds tested were found to inhibit human ROCK-2 and ROCK-1 kinases, exhibiting an IC₅₀ of about 10 μM to about 1 nM.

ii) In Vivo Data

Determination of Antinociceptive Effect: Models for Neuropathic Pain

Spinal Nerve (L5/L6) Ligation Model of Neuropathic Pain. As described in detail by Kim and Chung (Kim S. H.; Chung J. M. An experimental model for peripheral neuropathy produced by segmental spinal nerve ligation in the rat. Pain 1992, 50, 355-363), a 1.5 cm incision was made dorsal to the lumbosacral plexus. In anesthetized rats, the paraspinal muscles (left side) were separated from the spinous processes, the L5 and L6 spinal nerves isolated, and tightly ligated with 3-0 silk threads. Following hemostasis, the wound was sutured and coated with antibiotic ointment. The rats were allowed to recover and then placed in a cage with soft bedding for 14 days before behavioral testing for mechanical allodynia.

Sciatic Nerve Ligation Model of Neuropathic Pain. As described in details by Bennett and Xie (Bennett G. J.; and Xie Y-K., A peripheral mononeuropathy in rat that produces disorders of pain sensation like those seen in man. Pain, 1988, 33, 87-107), a 1.5 cm incision was made 0.5 cm below the pelvis and the biceps femoris of anesthetized rats, and the gluteous superficialis (right side) were separated. The sciatic nerve was exposed, isolated, and four loose ligatures (5-0 chromic catgut) with 1 mm spacing were placed around it. The rats were allowed to recover and then placed in a cage with soft bedding for 14 days before behavioral testing for mechanical allodynia as described above. In addition, animals were also tested for cold allodynia by dipping their hind paw in a cold-water bath (4.5° C.) and determining the paw withdrawal latency.

Selected analogs dosed either i.p. or p.o. demonstrated >30% inhibition of tactile allodynia in the Chung and Bennett models (Chaplan S R, Bach F W, Pogrel J W, Chung J M & Yaksh T L (1994). Quantitative assessment of tactile allodynia in the rat paw, Journal of Neuroscience Methods, 53(1):55-63.) of neuropathic pain at doses ranging from 1-150 mg/kg.

### d. Methods of using the Compounds

Compounds described herein have ROCK antagonistic activity.

Because of their profile, the compounds can be used for treating diseases which respond to the influencing of ROCK activity, i.e. they are effective for treating those medical disorders or diseases in which exerting an influence on (modulating) the ROCK activity leads to an improvement in the clinical picture or to the disease being cured. Examples of these diseases are given above.

The disorders which can be treated in accordance with the invention include the diseases listed in the Summary, e.g. cardiovascular diseases such as hypertension, chronic and congestive heart failure, cardiac hypertrophy, chronic renal failure, cerebral vasospasm after subarachnoid bleeding, pulmonary hypertension, and ocular hypertension; cancer and tumor metastasis, asthma; male erectile dysfunctions; female sexual dysfunctions; over-active bladder syndrome; preterm labor; ischemia reperfusion; myocardial infarction; restenosis; atherosclerosis; graft failure; CNS disorders, such as acute neuronal injury, e.g. spinal chord injury, traumatic brain injury, and stroke, Parkinson's disease, and Alzheimer's disease; inflammatory and demyelating diseases such as multiple sclerosis, acute and chronic pain, rheumatoid arthritis, osteoarthritis, osteoporosis, irritable bowel syndrome and inflammatory bowel disease, amyotrophic lateral sclerosis, HIV-1 encephalitis, virus and bacterial infections, insulin resistance, diabetes, cognitive dysfunctions, such as the above-mentioned Alzheimer's disease, vascular dementia and other dementia forms, glaucoma, psoriasis, retinopathy, and benign prostatic hypertrophy. In particular the disorders are cancer, pain (e.g. inflammatory pain, neuropath tic pain, nociceptive pain, cancer pain, and the like), asthma, cognitive dysfunctions, in particular vascular dementia and Alzheimer's disease, multiple sclerosis, rheumatoid arthritis and spinal cord injuries.

Within the meaning of the invention, a treatment also includes a preventive treatment (prophylaxis), in particular as relapse prophylaxis or phase prophylaxis, as well as the treatment of acute or chronic signs, symptoms and/or malfunctions. The treatment can be orientated symptomatically, for example as the suppression of symptoms. It can be effected over a short period, be orientated over the medium term or can be a long-term treatment, for example within the context of a maintenance therapy.

The treatment is effected by means of single or repeated daily administration, where appropriate together, or alternating, with other active compounds or active compound-containing preparations.

Within the context of the treatment, the use according to the invention of the described compounds involves a method. In this method, an effective quantity of one or more compounds, as a rule formulated in accordance with pharmaceutical and veterinary practice, is administered to the individual to be treated, preferably a mammal, in particular a human being, productive animal or domestic animal. Whether such a treatment is indicated, and in which form it is to take place, depends on the individual case and is subject to medical assessment (diagnosis) which takes into consideration signs, symptoms and/or malfunctions which are present, the risks of developing particular signs, symptoms and/or malfunctions, and other factors.

Present compounds can also be administered as a pharmaceutical composition including therapeutically effective amounts of the compounds of interest in combination with one or more pharmaceutically acceptable carriers. The phrase “therapeutically effective amount” of the present compounds means sufficient amount of the compounds to achieve the desired therapeutic response for a particular patient, compositions and mode of administration, at a reasonable benefit/risk ratio applicable to any medical treatment. It can be understood, however, that the total daily usage of the compounds and compositions can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds administered to a human or lower animal can range from about 0.003 to about 30 mg/kg/day. For purposes of oral administration, more preferable doses can be in the range of from about 0.01 to about 10 mg/kg/day. If desired, the effective daily dose can be divided into multiple doses for purposes of administration; consequently, single dose compositions can contain such amounts or submultiples thereof to make up the daily dose.

### e. Pharmaceutical Compositions

Further provided are pharmaceutical compositions capable of treating protein kinases associated conditions, in particular, Rho kinase (ROCK) mediated conditions, as described above. Pharmaceutical compositions including compounds of interest, or solvates or salts thereof can be formulated by employing conventional solid or liquid vehicles or diluents, as well as pharmaceutically acceptable additives of a type appropriate to the mode of administration (e.g. excipients, binders, preservatives, stabilizers, flavors, etc) according to techniques such as those well known in the art of pharmaceutical formulations.

The compounds described herein can be administered by any means suitable for the condition to be treated, which can depend on the need of site-specific treatment or quantity of drug to be delivered.

The pharmaceutical compositions can be administered to humans and other mammals orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection include pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It can also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound can be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form can also include buffering agents.

Solid compositions of a similar type can also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They can optionally contain opacifying agents and can also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring and perfuming agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Exemplary compositions for rectal or vaginal administration include suppositories which can be prepared by mixing the compounds of interest with suitable non-irritating carriers or carriers such as cocoa butter, polyethylene glycol or a suppository wax which are solid at room temperature but liquid at body temperature and therefore melt in the rectum or vaginal cavity and release the active compound.

Compounds described herein can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to the compounds of interest, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of the compounds include powders, sprays, ointments and inhalants. The active compound(s) can be mixed under sterile conditions with a pharmaceutically acceptable carrier and any needed preservatives, buffers or propellants which can be required. Opthalmic formulations, eye ointments, powders and solutions are also contemplated as being within the scope of this invention.

The compounds provided herein can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts are well known in the art. For example, S. M. Berge et al. describe pharmaceutically acceptable salts in detail in (J. Pharmaceutical Sciences, 1977, 66: 1 et seq). The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

The term “pharmaceutically acceptable prodrug” or “prodrug” as used herein, represents those prodrugs of the compounds which are, within the scope of sound medical judgment, suitable for use in contact with the tissues of humans and lower animals without undue toxicity, irritation, allergic response, and the like, commensurate with a reasonable benefit/risk ratio, and effective for their intended use.

The present application contemplates compounds formed by synthetic means or formed by in vivo biotransformation of a prodrug.

Compounds described herein can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### f. General Synthesis

This invention is intended to encompass compounds of the invention when prepared by synthetic processes or by metabolic processes. Preparation of the compounds by metabolic processes includes those occurring in the human or animal body (in vivo) or processes occurring in vitro.

The compounds provided herein can be prepared by a variety of processes well known for the preparation of compounds of this class. For example, compounds of formula (I) wherein the groups A, X¹, X², m, n, p, R¹, R², R³, and R⁴ have the meanings as set forth in the summary section unless otherwise noted, can be generally prepared as shown in Schemes 1-3.

As used in the descriptions of the schemes and the examples, certain abbreviations are intended to have the following meanings: HPLC for high performance liquid chromatography or high pressure liquid chromatography, dppf for [1,1′-bis(diphenylphosphino)ferrocene; DME for dimethoxyethane, DMSO for dimethylsulfoxide, triflate for trifluoromethylsulfonate; OMs or mesylate for methanesulfonate, tBu for tert-butyl, and OTs or tosylate for p-toluenesulfonate.

Compounds of general formula (I) wherein X¹ is C(O)NR⁴, C(O)O, or S(O)₂, can be prepared using the general procedure as outlined in Scheme 1.

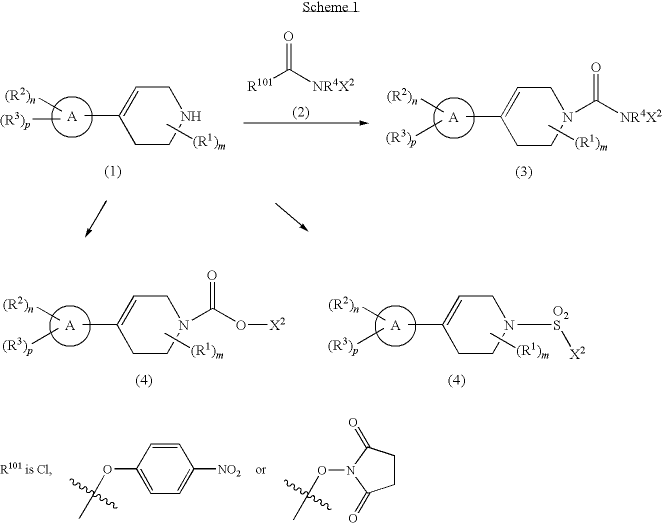

Compounds of formula (1) can be treated with isocyanates of formula X²NCO or reagents of formula (2) using reaction conditions that are known in the art to provide compounds of formula (3) wherein R⁴ is hydrogen. For example, the reaction can be conducted at ambient temperature in the presence of a base such as triethylamine. Compounds of formula (3) can also be prepared by treating (1) with an appropriate amine of formula X²N(H)(R⁴) in the presence of triphosgene, 4-nitrophenyl carbonochloridate, or bis(2,5-dioxopyrrolidin-1-yl)carbonate, and a base such as triethylamine to provide (3). While subjected to conditions known to those skilled in the art, compounds of formula (1) can be treated with (2) to provide (3).

Compounds of formula (4) can be obtained by treating compounds of formula (1) with chloroformates of formula ClC(O)OX² in the presence of a base such as triethylamine.

Compounds of formula (5) can be obtained by treating compounds of formula (1) with sulfonyl chlorides of formula X²S(O)₂Cl in the presence of a base such as triethylamine.

Compounds of formula (1) can also be treated with appropriate acid chlorides of formula X²C(O)Cl or acids of formula X²C(O)OH using reaction conditions that are known to one skilled in the art, to provide compounds of general formula (I) wherein X¹ is C(O).

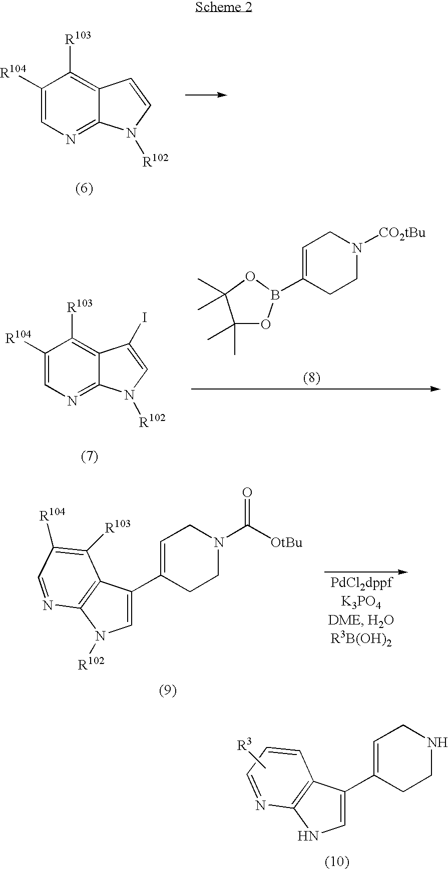

Intermediates of formula (10) can be prepared using the general procedure as illustrated in Scheme 2.

Cross coupling of compounds of formula (7) wherein one of R¹⁰³ and R¹⁰⁴ is Br and the other is hydrogen, and R¹⁰² is hydrogen or a protecting group such as, but not limited to, toluenesulfonyl, benzenesulfonyl or triisopropylsilyl, with the commercially available tetrahydropyridyl boronic ester of formula (8) in the presence of a palladium catalyst and a base such as potassium phosphate provides compounds of formula (9). Compounds of formula (7) wherein R¹⁰³ is hydrogen, R¹⁰⁴ is Br, and R¹⁰² is benzenesulfonyl has been described in WO2004/078756. Compounds of formula (7) can also be prepared using synthetic reactions that are well documented in the literature, for example, by iodination of (6) with iodine in the presence of potassium hydroxide.

Coupling of intermediates (9) wherein one of R¹⁰³ and R¹⁰⁴ is bromine with appropriate reagents under conditions known in the art introduces R³ (e.g. boronic acids or esters containing R³ functionality, alkynyl, OR^{a}, SR^{a}, NR^{a}R^{b}, CN, S(O)₂R^{c}) to the pyridyl ring.

Removal of the tert-butoxy carbonyl group on the tetrahydropyridine ring can be accomplished by treatment with an acid.

Removal of R¹⁰² can be achieved by various reaction conditions. For example, R¹⁰² is toluenesulfonyl or benzenesulfonyl can be removed by treatment with an hydroxide such as sodium hydroxide. Treatment with tetrabutylammonium fluoride would remove the triisopropylsilyl protecting group.

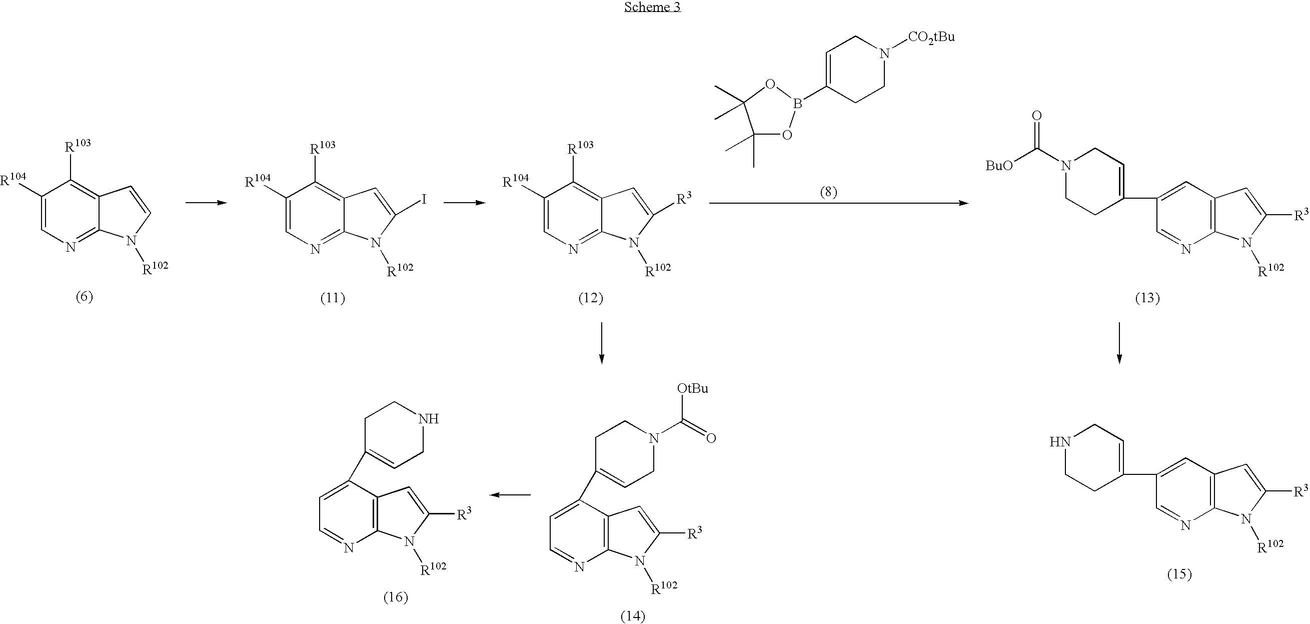

Intermediates of formula (15) and (16) can also be prepared using general procedures as outlined in Scheme 3.

Compounds of formula (6) wherein one of R¹⁰³ and R¹⁰⁴ is bromine and the other is hydrogen can be converted to (11) by treatment with lithium diisopropylamide at about −70° C., followed by addition of iodine. Compounds of formula (11) wherein R¹⁰² is toluenesulfonyl, R¹⁰³ is bromine, and R¹⁰⁴ is hydrogen have been disclosed in WO2003/000690. Cross coupling of (11) with boronic acids of formula R³B(OH)₂ under appropriate reaction conditions such as a palladium reagent, a ligand and optionally a base, provides compounds of formula (12). Reaction of (12) wherein R¹⁰³ is hydrogen and R¹⁰⁴ is bromine with (8), followed by stepwise removal of tert-butoxy carbonyl group and R¹⁰² using reaction conditions as described in Scheme 2 provides intermediates of formula (15).

Similarly, intermediates of formula (16) can be obtained from (12) wherein R¹⁰³ is bromine and R¹⁰⁴ is hydrogen after similar manipulation.

It can be appreciated that the synthetic schemes and specific examples as illustrated in the Examples section are illustrative and are not to be read as limiting the scope of the invention as it is defined in the appended claims. All alternatives, modifications, and equivalents of the synthetic methods and specific examples are included within the scope of the claims.

Optimum reaction conditions and reaction times for each individual step can vary depending on the particular reactants employed and substituents present in the reactants used. Unless otherwise specified, solvents, temperatures and other reaction conditions can be readily selected by one of ordinary skill in the art. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Routine experimentations, including appropriate manipulation of the reaction conditions, reagents and sequence of the synthetic route, protection of any chemical functionality that can not be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in T. Greene and P. Wuts, Protecting Groups in Chemical Synthesis (3^{rd} ed.), John Wiley & Sons, NY (1999), which is incorporated herein by reference in its entirety. Synthesis of the compounds of the invention can be accomplished by methods analogous to those described in the synthetic schemes described herein above and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the invention is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the invention is required, it can be obtained by carrying out one of the above procedures using a pure geometric isomer as a starting material, or by resolution of a mixture of the geometric isomers of the compound or intermediates using a standard procedure such as chromatographic separation.

The following Examples can be used for illustrative purposes and should not be deemed to narrow the scope of the invention.

### g. Examples

Products or intermediates that were purified by preparative HPLC were conducted on a Phenomenex Luna C8(2) 5 um 100 Å AXIA column (30 mm×75 mm). A gradient of acetonitrile (A) and 0.1% trifluoroacetic acid in water (B) was used, at a flow rate of 70 mL/min (0-0.5 min 10% A, 0.5-12.0 min linear gradient 10-95% A, 12.0-15.0 min 95% A, 15.0-17.0 min linear gradient 95-10% A). Samples were injected in 2.5mL dimethyl sullfoxide:methanol (1:1). A custom purification system was used, consisting of the following modules: Waters LC4000 preparative pump; Waters 996 diode-array detector; Waters 717+ autosampler; Waters SAT/IN module, Alltech Varex III evaporative light-scattering detector; Gilson 506C interface box; and two Gilson FC204 fraction collectors. The system was controlled using Waters Millennium32 software, automated using an Abbott developed Visual Basic application for fraction collector control and fraction tracking. Fractions were collected based upon UV signal threshold and selected fractions subsequently analyzed by flow injection analysis mass spectrometry using positive APCI ionization on a Finnigan LCQ using 70:30 methanol: 10 mM NH₄OH (aqueous) at a flow rate of 0.8 mL/min. Loop-injection mass spectra were acquired using a Finnigan LCQ running LCQ Navigator 1.2 software and a Gilson 215 liquid handler for fraction injection controlled by an Abbott developed Visual Basic application.

### Example 1

### N-[(1S)-2-hydroxy-1-phenylethyl]-4-(1H-pyrrolo[2,3-b]pyridin-3-yl)-3,6-dihydropyridine-1(2H)-carboxamide

### Example 1A

### (S)-2-(tert-butyldimethylsilyloxy)-1-phenylethanamine

A solution of (S)-2-amino-2-phenylethanol (1.04 g, 7.61 mmol), tert-butylchlorodimethylsilane (1.15 g, 7.62 mmol), triethylamine (2.15 mL, 15.4 mmol), N,N-dimethylpyridin-4-amine (23 mg, 0.19 mmol) in dichloromethane was stirred overnight at room temperature, quenched with saturated aqueous NaHCO₃, extracted with dichloromethane, dried (Na₂SO₄), filtered, and concentrated to give 1.85 g of clear oil, which was used without purification.

### Example 1B

### (S)-N-(2-(tert-butyldimethylsilyloxy)-1-phenylethyl)-4-(1H-pyrrolo[2,3-b]pyridin-3-yl)-5,6-dihydropyridine-1(2H)-carboxamide

A solution of the product from Example 1A (127 mg, 0.505 mmol), triphosgene (52.1 mg, 0.176 mmol), and triethylamine (0.25 mL, 1.8 mmol) in dichloromethane (2 mL) was stirred for 2 h at room temperature. 3-(1,2,3,6-tetrahydropyridin-4-yl)-1H-pyrrolo[2,3-b]pyridine (0.10 g, 0.50 mmol) was added and stirred for 2 h at room temperature. N,N-dimethylformamide (1 mL) was added for solubility, and the mixture was stirred overnight, diluted with ethyl acetate, washed with water and brine, dried (Na₂SO₄), filtered, concentrated and chromatographed (3% methanol/dichloromethane) to give the product as a clear gum (0.186 g, 0.391 mmol).

### Example 1C

### N-[(1S)-2-hydroxy-1-phenylethyl]-4-(1H-pyrrolo[2,3-b]pyridin-3-yl)-3,6-dihydropyridine-1(2H)-carboxamide

A solution of tetrabutylammonium fluoride (1 M in tetrahydrofuran, 0.50 mL, 0.50 mmol) was added to a solution of the product from Example IB in tetrahydrofuran (0.8 mL), stirred for 4 h at room temperature, concentrated, diluted with ethyl acetate, washed with water and brine, dried (Na₂SO₄), filtered, concentrated, and chromatographed (0-25% methanol/dichloromethane) to give the title compound as a tacky yellow solid (74 mg, 0.20 mmol). ¹H NMR (300 MHz, DMSO-d₆) δ ppm 11.65-11.68 (bs, 1H), 8.21-8.26 (m, 2H), 7.55 (d, J=2.3 Hz, 1H), 7.26-7.36 (m, 4H), 7.16-7.22 (m, 1H), 7.10 (dd, J=7.8, 4.8 Hz, 1H), 6.64 (d, J=7.7 Hz, 1H), 6.17-6.19 (bs, 1H), 4.74-4.83 (m, 2H), 4.05-4.11 (m, 2H), 3.49-3.66 (m, 4H), 2.46-2.54 (m, 2H); MS (ESI⁺) M/Z 363.0 (M+H)⁺.

### Example 2

### 3-[1-(3-phenylpropanoyl)-1,2,3,6-tetrahydropyridin-4-yl]-1H-pyrrolo[2,3-b]pyridine

A mixture of 3-(1,2,3,6-tetrahydropyridin-4-yl)-1H-pyrrolo[2,3-b]pyridine (99 mg, 0.49 mmol), triethylamine (0.090 mL, 0.65 mmol), and 3-phenylpropanoyl chloride (0.074 mL, 0.49 mmol) in N,N-dimethylformamide (1.5 mL) was stirred overnight at room temperature, diluted with ethyl acetate, washed with water and brine, dried (Na₂SO₄), filtered, concentrated, and chromatographed (60% ethyl acetate/dichloromethane) to give the title compound as a clear gum (78 mg, 0.23 mmol). ¹H NMR (300 MHz, DMSO-d₆) δ ppm 11.40 (bs, 1H), 8.21 (dd, J=1.2, 4.6 Hz, 1H), 8.17 (dd, J=1.2, 7.9 Hz, 1H), 7.46 (bs, 1H), 7.23-7.27 (m, 4H), 7.13-7.17 (m, 1H), 7.07 (dd, J=4.6, 7.9 Hz, 1H), 6.13 (bs, 1H), 4.15 (q, J=2.7 Hz, 2H), 3.68 (bs, 2H), 2.86-2.90 (m, 2H), 2.66-2.71 (t, J=7.5 Hz, 2H), 2.36 (bs, 2H); MS (ESI⁺) M/Z 332.0 (M+H)⁺.

### Example 3

### 3-{1-[(2-phenylethyl)sulfonyl]-1,2,3,6-tetrahydropyridin-4-yl}-1H-pyrrolo[2,3-b]pyridine

A mixture of 3-(1,2,3,6-tetrahydropyridin-4-yl)-1H-pyrrolo[2,3-b]pyridine (76 mg, 0.38 mmol), triethylamine (0.070 mL, 0.50 mmol), and 2-phenylethanesulfonyl chloride (86 mg, 0.42 mmol) in N,N-dimethylformamide (1.2 mL) was stirred for 1 h at room temperature, diluted with ethyl acetate, washed with water and brine, dried (Na₂SO₄), filtered, concentrated, and chromatographed (3% methanol/dichloromethane) to give the title compound as a white solid (57 mg, 0.15 mmol). ¹H NMR (300 MHz, DMSO-d₆) δ ppm 11.71 (bs, 1H), 8.21-8.27 (m, 2H), 7.57 (d, J=1.6 Hz, 1H), 7.28-7.32 (m, 4H), 7.17-7.24 (m, 1H), 7.10 (dd, J=4.8, 7.9 Hz, 1H), 6.22 (bs, 1H), 3.95-4.01 (m, 2H), 3.49 (t, J=5.8 Hz, 2H), 3.37-3.44 (m, 2H), 2.97-3.05 (m, 2H), 2.61 (bs, 2H); MS (ESI⁺) M/Z 367.9 (M+H)⁺.

### Example 4

### N-benzyl-4-(1H-pyrrolo[2,3-b]pyridin-3-yl)-3,6-dihydropyridine-1(2H)-carboxamide

A mixture of 3-(1,2,3,6-tetrahydropyridin-4-yl)-1H-pyrrolo[2,3-b]pyridine (80 mg, 0.40 mmol), triethylamine (0.056 mL, 0.40 mmol), and (isocyanatomethyl)benzene (0.049 mL, 0.40 mmol) in N,N-dimethylformamide (1.2 mL) was stirred for 90 min at room temperature, diluted with ethyl acetate, washed with water and brine, dried (Na₂SO₄), concentrated, filtered, and chromatographed (3 to 5% methanol/dichloromethane) to give the title compound as a yellow solid (82 mg, 0.25 mmol). ¹H NMR (500 MHz, DMSO-d₆) δ ppm 11.65-11.68 (bs, 1H), 8.23 (dd, J=8.0, 1.4 Hz, 1H), 8.21 (dd, J=4.7, 1.5 Hz, 1H), 7.55 (d, J=2.2 Hz, 1H), 7.25-7.31 (m, 4H), 7.17-7.21 (m, 1H), 7.10 (t, J=5.8 Hz, 1H), 7.08 (dd, J=8.0, 4.7 Hz, 1H), 6.17-6.19 (m, 1H), 4.28 (d, J=5.7 Hz, 2H), 4.04-4.07 (m, 2H), 3.59 (t, J=5.6 Hz, 2H), 2.48-2.52 (m, 2H); MS (ESI⁺) M/Z 333 (M+H)⁺.

### Example 5

### N-(1-naphthylmethyl)-4-(1H-pyrrolo[2,3-b]pyridin-3-yl)-3,6-dihydropyridine-1(2H)-carboxamide

The title compound was prepared using the procedure in Example 4 replacing (isocyanatomethyl)benzene with 1-(isocyanatomethyl)naphthalene. ¹H NMR (300 MHz, CDCl₃) δ ppm 8.98-9.11 (m, 1H), 8.31 (dd, J=4.7, 1.6 Hz, 1H), 8.11-8.15 (m, 2H), 7.89 (dd, J=7.5, 2.0 Hz, 1H), 7.82 (d, J=8.0 Hz, 1H), 7.42-7.61 (m, 4H), 7.27 (dd, J=8.4, 2.2 Hz, 1H), 7.11 (dd, J=8.0, 4.8 Hz, 1H), 6.07-6.11 (m, 1H), 4.95 (d, J=5.0 Hz, 2H), 4.69 (t, J=5.0 Hz, 1H), 4.03-4.07 (m, 2H), 3.72 (t, J=5.7 Hz, 2H), 2.55-2.63 (m, 2H); MS (ESI⁻) M/Z 383.0 (M+H)⁺.

### Example 6

### 3-{1-[(3-phenylmorpholin-4-yl)carbonyl]-1,2,3,6-tetrahydropyridin-4-yl}-1H-pyrrolo[2,3-b]pyridine

The title compound was prepared using the procedure in Example 1B replacing the product from Example 1A with 3-phenylmorpholine hydrochloride. ¹H NMR (300 MHz, DMSO-d₆) δ ppm 11.66-11.69 (bs, 1H), 8.21-8.27 (m, 2H), 7.54 (s, 1H), 7.34-7.40 (m, 2H), 7.27-7.35 (m, 2H), 7.18-7.25 (m, 1H), 7.10 (dd, J=7.9, 4.7 Hz, 1H), 6.19-6.22 (bs, 1H), 4.59 (t, J=4.0 Hz, 1H), 3.89-4.02 (m, 3H), 3.58-3.85 (m, 4H), 3.37-3.44 (m, 1H), 3.17-3.22 (m, 2H), 2.46-2.59 (m, 2H); MS (ESI⁻) M/Z 387 (M−H)⁻.

### Example 7

### 3-{1-[(4-methyl-2-phenylpiperazin-1-yl)carbonyl]-1,2,3,6-tetrahydropyridin-4-yl}-1H-pyrrolo[2,3-b]pyridine

The title compound was prepared using the procedure in Example 1B, replacing the product from Example 1A with 1-methyl-3-phenylpiperazine. ¹H NMR (300 MHz, DMSO-d₆) δ ppm 11.66-11.68 (bs, 1H), 8.21-8.27 (m, 2H), 7.54 (d, J=2.2 Hz, 1H), 7.35-7.39 (m, 2H), 7.25-7.32 (m, 2H), 7.15-7.22 (m, 1H), 7.09 (dd, J=7.9, 4.7 Hz, 1H), 6.19-6.22 (bs, 1H), 4.70-4.73 (m, 1H), 3.98-4.05 (m, 2H), 3.51-3.63 (m, 1H), 3.32-3.50 (m, 3H), 3.06-3.17 (m, 1H), 2.84-2.92 (m, 1H), 2.50-2.63 (m, 2H), 2.41-2.50 (m, 1H), 2.20-2.29 (m, 1H), 2.18 (s, 3H); MS (ESI⁻) M/Z 400 (M−H)⁻.

### Example 8

### N-[(1S)-1-phenylethyl]-4-(1H-pyrrolo[2,3-b]pyridin-3-yl)-3,6-dihydropyridine-1(2H)-carboxamide

The title compound was prepared using the procedure in Example 4 replacing (isocyanatomethyl)benzene with (S)-(1-isocyanatoethyl)benzene. ¹H NMR (300 MHz, DMSO-d₆) δ ppm 11.66-11.68 (bs, 1H), 8.21-8.26 (m, 2H), 7.54-7.56 (bs, 1H), 7.25-7.37 (m, 4H), 7.14-7.21 (m, 1H), 7.10 (dd, J=7.8, 4.8 Hz, 1H), 6.79 (d, J=7.9 Hz, 1H), 6.15-6.20 (m, 1H), 4.88 (p, J=7.3 Hz, 1H), 3.99-4.07 (m, 2H), 3.58 (t, J=5.7 Hz, 2H), 2.47-2.54 (m, 2H), 1.39 (d, J=7.0 Hz, 3H); MS (ESI⁺) M/Z 347 (M+H)⁺.

### Example 9

### N-[(1R)-1-phenylethyl]-4-(1H-pyrrolo[2,3-b]pyridin-3-yl)-3,6-dihydropyridine-1(2H)-carboxamide

The title compound was prepared using the procedure in Example 4 replacing (isocyanatomethyl)benzene with (R)-(1-isocyanatoethyl)benzene. ¹H NMR (300 MHz, DMSO-d₆) δ ppm 11.65-11.68 (bs, 1H), 8.21-8.25 (m, 2H), 7.55 (d, J=2.1 Hz, 1H), 7.26-7.36 (m, 4H), 7.15-7.21 (m, 1H), 7.09 (dd, J=7.7, 4.9 Hz, 1H), 6.78 (d, J=7.9 Hz, 1H), 6.16-6.18 (bs, 1H), 4.88 (p, J=7.3 Hz, 1H), 4.06 (m, 2H), 3.58 (t, J=5.7 Hz, 2H), 1.39 (d, J=7.1 Hz, 3H); MS (ESI⁺) M/Z 347 (M+H)⁻.

### Example 10

### N-(2-phenoxyethyl)-4-(1H-pyrrolo[2,3-b]pyridin-3-yl)-3,6-dihydropyridine-1(2H)-carboxamide

The title compound was prepared using the procedure in Example 1B replacing the product from Example 1A with 2-phenoxyethanamine. ¹H NMR (300 MHz, DMSO-d₆) δ ppm 11.65-11.68 (bs, 1H), 8.20-8.26 (m, 2H), 7.54 (d, J=2.5 Hz, 1H), 7.24-7.30 (m, 2H), 7.09 (dd, J=7.8, 4.8 Hz, 1H), 6.88-7.01 (m, 3H), 6.75 (t, J=5.4 Hz, 1H), 6.14-6.20 (m, 1H), 3.97-4.04 (m, 4H), 3.56 (t, J=5.3 Hz, 2H), 3.36-3.45 (m, 2H); MS (ESI⁺) M/Z 363 (M+H)⁺.

### Example 11

### N-(2-phenylethyl)-4-(1H-pyrrolo[2,3-b]pyridin-3-yl)-3,6-dihydropyridine-1(2H)-carboxamide

To a solution of 3-(1,2,3,6-tetrahydropyridin-4-yl)-1H-pyrrolo[2,3-b]pyridine (25 mg, 0.13 mmol) in 1.0 mL dimethylacetamide was added a solution of the monomer (2-isocyanatoethyl)benzene (22 mg, 0.15 mmol, 1.2 eq.) in 0.5 mL dimethylacetamide. The mixture was shaken overnight at room temperature, concentrated, and purified by preparative HPLC on a Phenomenex Luna C8(2) 5 μm 100 < AXIA column (30 mm×75 mm) using a gradient of acetonitrile (A) and 0.1% trifluoroacetic acid in water (B), at a flow rate of 50 mL/min (0-0.5 min 10% A, 0.5-6.0 min linear gradient 10-100% A, 6.0-7.0 min 100% A, 7.0-8.0 min linear gradient 100-10% A) to give the trifluoroacetic acid salt of the title compound as a tan solid. ¹H NMR (300 MHz, DMSO-d₆/Deuterium Oxide, Temp=120 C) δ ppm 8.21 (dd, J=4.7, 1.6 Hz, 1H), 8.16 (dd, J=8.0, 1.6 Hz, 1H), 7.43 (s, 1H), 7.13-7.29 (m, 5H), 7.09 (dd, J=8.0, 4.7 Hz, 1H), 6.10-6.13 (m, 1H), 3.98-4.02 (m, 2H), 3.55 (t, J=5.7 Hz, 2H), 3.31-3.38 (m, 2H), 2.75-2.82 (m, 2H), 2.47-2.54 (m, 2H); MS (ESI⁻) M/Z 345 (M−H)⁻.

### Example 12

### N-(2,4-dichlorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 2,4-dichloro-1-(isocyanatomethyl)benzene. ¹H NMR (300 MHz, DMSO-d₆/Deuterium Oxide, Temp=120 C) δ ppm 8.21 (dd, J=4.7, 1.6 Hz, 1H), 8.17 (dd, J=8.0, 1.6 Hz, 1H), 7.44-7.45 (bs, 2H), 7.39 (d, J=8.3 Hz, 1H), 7.32 (dd, J=8.3, 2.1 Hz, 1H), 7.09 (dd, J=8.0, 4.7 Hz, 1H), 6.13-6.16 (m, 1H), 4.37 (s, 2H), 4.06-4.10 (m, 2H), 3.62 (t, J=5.7 Hz, 2H), 2.54-2.57 (m, 2H); MS (ESI⁻) M/Z 399 (M−H)⁻.

### Example 13

### N-(2-chlorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-chloro-2-(isocyanatomethyl)benzene. ¹H NMR (300 MHz, DMSO-d₆/Deuterium Oxide, Temp=120 C) δ ppm 8.21 (dd, J=4.7, 1.5 Hz, 1H), 8.18 (dd, J=8.0, 1.6 Hz, 1H), 7.44 (s, 1H), 7.34-7.41 (m, 2H), 7.19-7.31 (m, 2H), 7.09 (dd, J=8.0, 4.7 Hz, 1H), 6.13-6.16 (m, 1H), 4.41 (s, 2H), 4.07-4.11 (m, 2H), 3.63 (t, J=5.7 Hz, 2H), 2.52-2.59 (m, 2H); MS (ESI⁻) M/Z 365 (M−H)⁻.

### Example 14

### N-(3 4-dichlorobenzyl)-4-(1H-pyrrolo[2,3-b]pyridin-3-yl)-3 6-dihydropyridine-1(2H)-carboxamide

The trifluoroacetic acid salt of the title compound was prepared using the procedure in Example 11 replacing (2-isocyanatoethyl)benzene with 1,2-dichloro-4-(isocyanatomethyl)benzene. ¹H NMR (300 MHz, DMSO-d₆/Deuterium Oxide, Temp=120 C) δ ppm 8.21 (dd, J=4.7, 1.5 Hz, 1H), 8.17 (dd, J=8.0, 1.6 Hz, 1H), 7.46-7.50 (m, 2H), 7.44 (s, 1H), 7.25-7.29 (m, 1H), 7.09 (dd, J=8.0, 4.7 Hz, 1H), 6.12-6.15 (m, 1H), 4.28 (s, 2H), 4.06 (q, J=2.8 Hz, 2H), 3.60 (t, J=5.7 Hz, 2H), 2.55 (d, J=4.4 Hz, 2H); MS (ESI⁺) M/Z 401 (M+H)⁺.

### Example 15

### N-(4-fluorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-fluoro-4-(isocyanatomethyl)benzene. ¹H NMR (300 MHz, DMSO-d₆/Deuterium Oxide, Temp=120 C) δ ppm 8.21 (dd, J=4.6, 1.6 Hz, 1H), 8.17 (dd, J=7.9, 1.6 Hz, 1H), 7.43 (s, 1H), 7.32 (dd, J=8.4, 5.5 Hz, 2H), 7.01-7.11 (m, 3H), 6.12-6.15 (m, 1H), 4.28-4.29 (bs, 2H), 4.01-4.07 (m, 2H), 3.60 (t, J=5.7 Hz, 2H), 2.50-2.56 (m, 2H); MS (APCI+) M/Z 351 (M+H)⁺.

### Example 16

### N-(4-methoxybenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(isocyanatomethyl)-4-methoxybenzene. ¹H NMR (300 MHz, DMSO-d₆/Deuterium Oxide, Temp=120 C) δ ppm 8.15-8.28 (m, 2H), 7.34-7.44 (m, 1H), 7.20-7.23 (m, 2H), 7.09 (dd, J=7.9, 4.6 Hz, 1H), 6.82-6.86 (m, 2H), 6.13 (d, J=3.5 Hz, 1H), 4.24 (s, 2H), 4.05 (q, J=2.7 Hz, 2H), 3.73 (s, 3H), 3.59 (t, J=5.7 Hz, 2H), 2.51-2.56 (m, 2H) ; MS (APCI+) M/Z 363 (M+H)⁺.

### Example 17

### N-(3-methylbenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(isocyanatomethyl)-3-methylbenzene. ¹H NMR (300 MHz, DMSO-d₆/Deuterium Oxide, Temp=120 C) δ ppm 8.21 (dd, J=4.7, 1.6 Hz, 1H), 8.17 (dd, J=8.0, 1.6 Hz, 1H), 7.43 (s, 1H), 7.06-7.19 (m, 4H), 7.01 (d, J=7.5 Hz, 1H), 6.12-6.15 (m, 1H), 4.27 (s, 2H), 4.06 (t, J=2.9 Hz, 2H), 3.61 (t, J=5.7 Hz, 2H), 2.50-2.58 (m, 2H), 2.27 (s, 3H); MS (ESI⁻) M/Z 345 (M−H)⁻.

### Example 18

### N-(4-methylbenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(isocyanatomethyl)-4-methylbenzene. ¹H NMR (300 MHz, DMSO-d₆/Deuterium Oxide, Temp=120 C) δ ppm 8.21 (dd, J=4.7, 1.6 Hz, 1H), 8.17 (dd, J=8.1, 1.5 Hz, 1H), 7.43 (s, 1H), 7.15-7.20 (m, 2H), 7.07-7.14 (m, 3H), 6.11-6.14 (m, 1H), 4.26 (s, 2H), 4.05 (q, J=2.8 Hz, 2H), 3.60 (t, J=5.7 Hz, 2H), 2.51-2.55 (m, 2H), 2.26 (s, 3H); MS (ESI⁻) M/Z 345 (M−H)⁻.

### Example 19

### N-(2-methylbenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(isocyanatomethyl)-2-methylbenzene. ¹H NMR (300 MHz, DMSO-d₆/Deuterium Oxide, Temp=120 C) δ ppm 8.21 (dd, J=4.7, 1.4 Hz, 1H), 8.17 (dd, J=8.0, 1.6 Hz, 1H), 7.43 (s, 1H), 7.07-7.28 (m, 5H), 6.12-6.15 (m, 1H), 4.31 (s, 2H), 4.05-4.09 (m, 2H), 3.61 (t, J=5.7 Hz, 2H), 2.52-2.57 (m, 2H), 2.30 (s, 3H); MS (ESI⁻) M/Z 345 (M−H)⁻.

### Example 20

### N-(4-bromobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-bromo-4-(isocyanatomethyl)benzene. ¹H NMR (300 MHz, DMSO-d₆/Deuterium Oxide, Temp=120 C) δ ppm 8.16-8.25 (m, 2H), 7.37-7.46 (m, 3H), 7.22-7.27 (m, 2H), 7.10 (dd, J=8.0, 4.7 Hz, 1H), 6.13 (dd, J=3.7, 2.0 Hz, 1H), 4.27 (s, 2H), 4.04-4.08 (m, 2H), 3.60 (t, J=5.8 Hz, 2H), 2.51-2.56 (m, 2H); MS (ESI⁺) M/Z 411 (M+H)⁺.

### Example 21

### N-(2-fluorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-fluoro-2-(isocyanatomethyl)benzene. ¹H NMR (300 MHz, DMSO-d₆/Deuterium Oxide, Temp=120 C) δ ppm 8.16-8.23 (m, 2H), 7.43-7.45 (bs, 1H), 7.31-7.41 (m, 1H), 7.03-7.28 (m, 4H), 6.12-6.16 (bs, 1H), 4.36-4.38 (bs, 2H), 4.05-4.09 (m, 2H), 3.61 (t, J=5.7 Hz, 2H), 2.50-2.57 (m, 2H); MS (APCI+) M/Z 351 (M+H)⁺.

### Example 22

### N-(3-fluorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-fluoro-3-(isocyanatomethyl)benzene. ¹H NMR (300 MHz, DMSO-d₆/Deuterium Oxide, Temp=120 C) δ ppm 8.16-8.23 (m, 2H), 7.44 (s, 1H), 7.24-7.35 (m, 1H), 7.04-7.15 (m, 3H), 6.93-7.02 (m, 1H), 6.13-6.15 (m, 1H), 4.32 (s, 2H), 4.06-4.09 (m, 2H), 3.61 (t, J=5.7 Hz, 2H), 2.51-2.57 (m, 2H); MS (ESI⁻) M/Z 349 (M−H)⁻.

### Example 23

### 4-(1H-pyrrolo[2,3-b]pyridin-3-yl)-N-(3,4,5-trimethoxybenzyl)-3,6-dihydropyridine-1(2H)-carboxamide

The trifluoroacetic acid salt of the title compound was prepared using the procedure in Example 11 replacing (2-isocyanatoethyl)benzene with 5-(isocyanatomethyl)-1,2,3-trimethoxybenzene. ¹H NMR (300 MHz, DMSO-d₆/Deuterium Oxide, Temp=120 C) δ ppm 8.20-8.27 (m, 2H), 7.45-7.55 (m, 1H), 7.12-7.17 (m, 1H), 6.61 (s, 2H), 6.15-6.17 (bs, 1H), 4.24 (s, 2H), 4.06-4.10 (m, 2H), 3.75 (s, 6H), 3.67 (s, 3H), 3.62 (t, J=5.7 Hz, 2H), 2.51-2.58 (m, 2H); MS (ESI⁺) M/Z 423.2 (M+H)⁺.

### Example 24

### N-(2-methoxybenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(isocyanatomethyl)-2-methoxybenzene. ¹H NMR (300 MHz, DMSO-d₆/Deuterium Oxide, Temp=120 C) δ ppm 8.19-8.24 (m, 2H), 7.45 (s, 1H), 7.11-7.25 (m, 3H), 6.85-6.99 (m, 2H), 6.12-6.18 (m, 1H), 4.31 (s, 2H), 4.02-4.08 (m, 2H), 3.82 (s, 3H), 3.60 (t, J=5.8 Hz, 2H), 2.51-2.58 (m, 2H) ; MS (APCI+) M/Z 363 (M+H)⁺.

### Example 25

### N-(2-ethoxybenzyl)-4-(1H-pyrrolo[2,3-b]pyridin-3-yl)-3 6-dihydropyridine-1(2H)-carboxamide

The trifluoroacetic acid salt of the title compound was prepared using the procedure in Example 11 replacing (2-isocyanatoethyl)benzene with 1-ethoxy-2-(isocyanatomethyl)benzene. ¹H NMR (300 MHz, DMSO-d₆/Deuterium Oxide, Temp=120 C) δ ppm 8.15-8.25 (m, 2H), 7.44 (s, 1H), 7.13-7.25 (m, 2H), 7.06-7.12 (m, 1H), 6.93 (dd, J=8.1, 1.1 Hz, 1H), 6.84-6.90 (m, 1H), 6.12-6.16 (m, 1H), 4.32 (s, 2H), 4.08 (q, J=6.9 Hz, 2H), 4.05-4.09 (m, 2H), 3.61 (t, J=5.7 Hz, 2H), 2.50-2.59 (m, 2H), 1.36 (t, J=6.9 Hz, 3H); MS (ESI⁻) M/Z 375 (M−H)⁻.

### Example 26

### N-(3-methoxybenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(isocyanatomethyl)-3-methoxybenzene. ¹H NMR (300 MHz, DMSO-d₆/Deuterium Oxide, Temp=120 C) δ ppm 8.16-8.26 (m, 2H), 7.44 (s, 1H), 7.16-7.22 (m, 1H), 7.07-7.14 (m, 1H), 6.86-6.90 (m, 2H), 6.74-6.79 (m, 1H), 6.12-6.16 (m, 1H), 4.28 (s, 2H), 4.05-4.09 (m, 2H), 3.73 (s, 3H), 3.61 (t, J=5.7 Hz, 2H), 2.51-2.57 (m, 2H); MS (ESI⁻) M/Z 361 (M−H)⁻.

### Example 27

### N-[2-(1,3-benzodioxol-5-yl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 5-(2-isocyanatoethyl)benzo[d][1,3]dioxole. ¹H NMR (300 MHz, DMSO-d₆/Deuterium Oxide, Temp=120 C) δ ppm 8.14-8.25 (m, 2H), 7.43 (s, 1H), 7.07-7.15 (m, 1H), 6.73-6.81 (m, 2H), 6.65-6.69 (m, 1H), 6.10-6.14 (m, 1H), 5.88-5.94 (m, 2H), 3.98-4.01 (m, 2H), 3.55 (t, J=5.7 Hz, 2H), 3.27-3.33 (m, 2H), 2.70 (t, J=7.3 Hz, 2H), 2.47-2.53 (m, 2H); MS (ESI⁻) M/Z 389 (M−H)⁻.

### Example 28

### N-[2-(3,5-dimethoxyphenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(2-isocyanatoethyl)-3,5-dimethoxybenzene. ¹H NMR (300 MHz, DMSO-d₆/Deuterium Oxide, Temp=120 C) δ ppm 8.17-8.26 (m, 2H), 7.44 (d, J=1.9 Hz, 1H), 7.08-7.15 (m, 1H), 6.38-6.43 (m, 2H), 6.30-6.32 (m, 1H), 6.10-6.15 (m, 1H), 3.99-4.02 (m, 2H), 3.72 (s, 6H), 3.55 (t, J=5.7 Hz, 2H), 3.31-3.39 (m, 2H), 2.72 (t, J=7.3 Hz, 2H), 2.47-2.54 (m, 2H); MS (ESI⁺) M/Z 407.1 (M+H)⁺.

### Example 29

### N-[2-(2,3-dimethoxyphenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(2-isocyanatoethyl)-2,3-dimethoxybenzene. ¹H NMR (300 MHz, DMSO-d₆/Deuterium Oxide, Temp=120 C) δ ppm 8.18-8.25 (m, 2H), 7.44 (s, 1H), 7.09-7.15 (m, 1H), 6.93 (dd, J=8.2, 7.5 Hz, 1H), 6.86 (dd, J=8.1, 1.8 Hz, 1H), 6.79 (dd, J=7.5, 1.8 Hz, 1H), 6.10-6.14 (m, 1H), 3.98-4.03 (m, 2H), 3.76-3.82 (m, 6H), 3.55 (t, J=5.7 Hz, 2H), 3.31 (s, 2H), 2.75-2.81 (m, 2H), 2.46-2.53 (m, 2H); MS (ESI⁺) M/Z 407.2 (M+H)⁺.

### Example 30

### N-[2-(3 4-dichlorophenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1,2-dichloro-4-(2-isocyanatoethyl)benzene. ¹H NMR (300 MHz, DMSO-d₆/Deuterium Oxide, Temp=120 C) δ ppm 8.21 (dd, J=4.7, 1.5 Hz, 1H), 8.16 (dd, J=7.9, 1.5 Hz, 1H), 7.40-7.45 (m, 3H), 7.16-7.20 (m, 1H), 7.09 (dd, J=8.0, 4.7 Hz, 1H), 6.09-6.13 (m, 1H), 3.97-4.01 (m, 2H), 3.54 (t, J=5.7 Hz, 2H), 3.35 (t, J=7.1 Hz, 2H), 2.79 (t, J=7.1 Hz, 2H), 2.47-2.53 (m, 2H); MS (ESI⁻) M/Z 413 (M−H)⁻.

### Example 31

### N-[2-(2,6-dichlorophenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1,3-dichloro-2-(2-isocyanatoethyl)benzene. ¹H NMR (300 MHz, DMSO-d₆/Deuterium Oxide, Temp=120 C) δ ppm 8.15-8.22 (m, 2H), 7.42 (s, 1H), 7.34-7.38 (m, 2H), 7.22 (dd, J=8.7, 7.2 Hz, 1H), 7.07-7.12 (m, 1H), 6.09-6.13 (m, 1H), 3.98-4.02 (m, 2H), 3.55 (t, J=5.7 Hz, 2H), 3.34-3.40 (m, 2H), 3.09-3.16 (m, 2H), 2.47-2.53 (m, 2H); MS (ESI⁻) M/Z 413 (M−H)⁻.

### Example 32

### N-[2-(5-bromo-2-methoxyphenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 4-bromo-2-(2-isocyanatoethyl)-1-methoxybenzene. ¹H NMR (300 MHz, DMSO-d₆/Deuterium Oxide, Temp=120 C) δ ppm 8.20-8.22 (m, 1H), 8.16 (dd, J=7.9, 1.6 Hz, 1H), 7.43 (s, 1H), 7.23-7.33 (m, 2H), 7.09 (dd, J=7.9, 4.7 Hz, 1H), 6.90 (d, J=8.4 Hz, 1H), 6.09-6.14 (m, 1H), 3.97-4.03 (m, 2H), 3.79 (s, 3H), 3.54 (t, J=5.7 Hz, 2H), 3.30 (t, J=7.0 Hz, 2H), 2.76 (t, J=7.1 Hz, 2H), 2.45-2.54 (m, 2H); MS (ESI⁻) M/Z 453 (M−H)⁻.

### Example 33

### N-[2-(3-bromo-4-methoxyphenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 2-bromo-4-(2-isocyanatoethyl)-1-methoxybenzene. ¹H NMR (300 MHz, DMSO-d₆/Deuterium Oxide, Temp=120 C) δ ppm 8.15-8.25 (m, 2H), 7.43 (s, 1H), 7.38 (d, J=2.1 Hz, 1H), 7.15-7.24 (m, 1H), 7.06-7.13 (m, 1H), 6.98 (d, J=8.3 Hz, 1H), 6.10-6.13 (m, 1H), 3.98-4.01 (m, 2H), 3.79 (s, 3H), 3.55 (t, J=5.7 Hz, 2H), 3.31 (t, J=7.2 Hz, 2H), 2.72 (t, J=7.2 Hz, 2H), 2.46-2.53 (m, 2H) ; MS (ESI⁻) M/Z 453 (M−H)⁻.

### Example 34

### N-[2-(2,5-dimethoxyphenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 2-(2-isocyanatoethyl)-1,4-dimethoxybenzene. ¹H NMR (300 MHz, DMSO-d₆/Deuterium Oxide, Temp=120 C) δ ppm 8.20-8.28 (m, 2H), 7.45-7.47 (m, 1H), 7.11-7.18 (m, 1H), 6.86 (d, J=8.5 Hz, 1H), 6.70-6.79 (m, 2H), 6.10-6.15 (m, 1H), 3.98-4.02 (m, 2H), 3.75 (s, 3H), 3.68 (s, 3H), 3.55 (t, J=5.7 Hz, 2H), 3.27-3.34 (m, 2H), 2.76 (t, J=7.2 Hz, 2H), 2.46-2.54 (m, 2H) ; MS (ESI⁺) M/Z 407 (M+H)⁺.

### Example 35

### N-(4-chlorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-chloro-4-(isocyanatomethyl)benzene. ¹H NMR (300 MHz, DMSO-d₆/Deuterium Oxide, Temp=120 C) δ ppm 8.14-8.22 (m, 2H), 7.44 (s, 1H), 7.43-7.43 (bs, 1H), 7.29 (s, 4H), 7.06-7.11 (m, 1H), 4.28 (s, 2H), 4.04-4.07 (m, 2H), 3.59 (t, J=5.8 Hz, 2H), 2.47-2.55 (m, 2H); MS (ESI⁻) M/Z 365 (M−H)⁻.

### Example 36

### N-[2-(2-fluorophenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-fluoro-2-(2-isocyanatoethyl)benzene. ¹H NMR (300 MHz, DMSO-d₆/Deuterium Oxide, Temp=120 C) δ ppm 8.15-8.26 (m, 2H), 7.43 (s, 1H), 7.16-7.37 (m, 2H), 7.01-7.16 (m, 3H), 6.10-6.15 (m, 1H), 3.98-4.02 (m, 2H), 3.55 (t, J=5.8 Hz, 2H), 3.33-3.39 (m, 2H), 2.78-2.86 (m, 2H), 2.47-2.54 (m, 2H); MS (ESI⁻) M/Z 363 (M−H)⁻.

### Example 37

### N-[2-(4-methoxyphenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-(2-isocyanatoethyl)-4-methoxybenzene. ¹H NMR (300 MHz, DMSO-d₆/Deuterium Oxide, Temp=120 C) δ ppm 8.16-8.22 (m, 2H), 7.43 (s, 1H), 7.07-7.18 (m, 3H), 6.79-6.86 (m, 2H), 6.10-6.13 (m, 1H), 3.97-4.01 (m, 2H), 3.71 (s, 3H), 3.52-3.57 (m, 2H), 3.28-3.33 (m, 2H), 2.69-2.75 (m, 2H), 2.47-2.53 (m, 2H); MS (ESI⁻) M/Z 375 (M−H)⁻.

### Example 38

### N-[2-(3-chlorophenyl)ethyl]-4-(1H-pyrrolo[2,3-b]pyridin-3-yl)-3 6-dihydropyridine-1(2H)-carboxamide

The trifluoroacetic acid salt of the title compound was prepared using the procedure in Example 11 replacing (2-isocyanatoethyl)benzene with 1-chloro-3-(2-isocyanatoethyl)benzene. ¹H NMR (300 MHz, DMSO-d₆/Deuterium Oxide, Temp=120 C) δ ppm 8.21 (dd, J=4.7, 1.6 Hz, 1H), 8.16 (dd, J=8.0, 1.6 Hz, 1H), 7.43 (s, 1H), 7.14-7.31 (m, 4H), 7.09 (dd, J=7.9, 4.7 Hz, 1H), 6.09-6.13 (m, 1H), 3.98-4.01 (m, 2H), 3.55 (t, J=5.7 Hz, 2H), 3.31-3.38 (m, 2H), 2.79 (t, J=7.3 Hz, 2H), 2.47-2.54 (m, 2H); MS (ESI⁻) M/Z 379 (M−H)⁻.

### Example 39

### N-[2-(2,4-dichlorophenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 2,4-dichloro-1-(2-isocyanatoethyl)benzene. ¹H NMR (300 MHz, DMSO-d₆/Deuterium Oxide, Temp=120 C) δ ppm 8.21 (dd, J=4.7, 1.6 Hz, 1H), 8.16 (dd, J=8.0, 1.6 Hz, 1H), 7.29-7.43 (m, 2H), 7.33 (d, J=8.2 Hz, 1H), 7.26 (dd, J=8.2, 2.1 Hz, 1H), 7.09 (dd, J=7.9, 4.6 Hz, 1H), 6.09-6.13 (m, 1H), 3.97-4.01 (m, 2H), 3.54 (t, J=5.7 Hz, 2H), 3.37 (t, J=7.1 Hz, 2H), 2.91 (t, J=7.1 Hz, 2H), 2.47-2.54 (m, 2H); MS (ESI⁻) M/Z 413 (M−H)⁻.

### Example 40

### N-[2-(4-fluorophenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-fluoro-4-(2-isocyanatoethyl)benzene. ¹H NMR (300 MHz, DMSO-d₆/Deuterium Oxide, Temp=120 C) δ ppm 8.20-8.25 (m, 2H), 7.45 (s, 1H), 7.19-7.29 (m, 2H), 7.08-7.18 (m, 1H), 6.97-7.06 (m, 2H), 6.10-6.15 (m, 1H), 3.98-4.01 (m, 2H), 3.54 (t, J=5.7 Hz, 2H), 3.29-3.37 (m, 2H), 2.75-2.81 (m, 2H), 2.48-2.54 (m, 2H); MS (ESI⁻) M/Z, 399 (M+Cl)−.

### Example 41

### N-(2,2-diphenylethyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with (2-isocyanatoethane-1,1-diyl)dibenzene. ¹H NMR (300 MHz, DMSO-d₆/Deuterium Oxide, Temp=120 C) δ ppm 8.20 (dd, J=4.6, 1.5 Hz, 1H), 8.13 (dd, J=7.9, 1.5 Hz, 1H), 7.39 (s, 1H), 7.25-7.28 (m, 8H), 7.12-7.20 (m, 2H), 7.08 (dd, J=8.0, 4.7 Hz, 1H), 6.02-6.07 (m, 1H), 4.31 (t, J=7.8 Hz, 1H), 3.88 (d, J=3.3 Hz, 2H), 3.75 (d, J=7.9 Hz, 2H), 3.45 (t, J=5.7 Hz, 2H), 2.38-2.44 (m, 2H); MS (ESI⁻) M/Z 421 (M−H)⁻.

### Example 42

### N-[2-(3,4-dimethoxyphenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 4-(2-isocyanatoethyl)-1,2-dimethoxybenzene. ¹H NMR (300 MHz, DMSO-d₆/Deuterium Oxide, Temp=120 C) δ ppm 8.22-8.28 (m, 2H), 7.46 (s, 1H), 7.12-7.18 (m, 1H), 6.81-6.86 (m, 2H), 6.74 (dd, J=8.1, 2.0 Hz, 1H), 6.14 (d, J=3.5 Hz, 1H), 3.99-4.02 (m, 2H), 3.75 (s, 3H), 3.72 (s, 3H), 3.52-3.58 (m, 2H), 3.29-3.36 (m, 2H), 2.69-2.75 (m, 2H), 2.47-2.54 (m, 2H); MS (ESI⁺) M/Z 407 (M+H)⁺.

### Example 43

### N-[2-(4-chlorophenyl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 1-chloro-4-(2-isocyanatoethyl)benzene. ¹H NMR (300 MHz, DMSO-d₆/Deuterium Oxide, Temp=120 C) δ ppm 8.15-8.25 (m, 2H), 7.36-7.44 (m, 1H), 7.20-7.30 (m, 4H), 7.07-7.15 (m, 1H), 6.10-6.15 (m, 1H), 3.98-4.01 (m, 2H), 3.54 (t, J=5.7 Hz, 2H), 3.30-3.37 (m, 2H), 2.74-2.82 (m, 2H), 2.47-2.53 (m, 2H); MS (ESI⁻) M/Z 379 (M−H)⁻.

### Example 44

### N-(cyclohexylmethyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with (isocyanatomethyl)cyclohexane. ¹H NMR (300 MHz, DMSO-d₆/Deuterium Oxide, Temp=120 C) δ ppm 8.19-8.22 (m, 1H), 8.16 (dd, J=8.0, 1.6 Hz, 1H), 7.43 (s, 1H), 7.09 (dd, J=7.9, 4.7 Hz, 1H), 6.10-6.14 (m, 1H), 4.00-4.03 (m, 2H), 3.56 (t, J=5.7 Hz, 2H), 2.97 (d, J=6.7 Hz, 2H), 2.48-2.55 (m, 2H), 1.56-1.76 (m, 5H), 1.38-1.55 (m, 1H), 1.11-1.27 (m, 3H), 0.88-0.98 (m, 2H) ; MS (ESI⁻) M/Z 337 (M−H)⁻.

### Example 45

### N-(4-phenylbutyl)-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with (4-isocyanatobutyl)benzene. ¹H NMR (300 MHz, DMSO-d₆/Deuterium Oxide, Temp=120 C) δ ppm 8.21 (dd, J=4.7, 1.6 Hz, 1H), 8.16 (dd, J=7.9, 1.6 Hz, 1H), 7.42 (s, 1H), 7.11-7.27 (m, 5H), 7.09 (ddd, J=8.0, 4.7, 0.3 Hz, 1H), 6.10-6.15 (m, 1H), 3.99-4.03 (m, 2H), 3.55 (t, J=5.7 Hz, 2H), 3.13 (t, J=6.9 Hz, 2H), 2.60 (t, J=7.5 Hz, 2H), 2.48-2.55 (m, 2H), 1.56-1.68 (m, 2H), 1.44-1.56 (m, 2H); MS (ESI⁻) M/Z 373 (M−H)⁻.

### Example 46

### N-[(1,1-dioxidotetrahydrothien-3-yl)methyl]-4-(1H-pyrrolo[2,3-b]pyridin-3-yl)-3,6-dihydropyridine-1(2H)-carboxamide

The trifluoroacetic acid salt of the title compound was prepared using the procedure in Example 11 replacing (2-isocyanatoethyl)benzene with 3-(isocyanatomethyl)-tetrahydrothiophene-1,1-dioxide. ¹H NMR (300 MHz, DMSO-d₆/Deuterium Oxide, Temp=120 C) δ ppm 8.23-8.27 (m, 2H), 7.47-7.56 (m, 1H), 7.13-7.20 (m, 1H), 6.13-6.17 (m, 1H), 4.02-4.05 (m, 2H), 3.58 (t, J=5.7 Hz, 1H), 3.25 (dd, J=6.5, 3.3 Hz, 2H), 3.08-3.13 (m, 2H), 2.93-3.05 (m, 2H), 2.76-2.86 (m, 1H), 2.57-2.72 (m, 1H), 2.50-2.56 (m, 2H), 2.17-2.29 (m, 1H), 1.79-1.93 (m, 1H); MS (ESI⁺) M/Z 375.1 (M+H)⁺.

### Example 47

### 4-(1H-pyrrolo[2,3-b]pyridin-3-yl)-N-(2-thien-2-ylethyl)-3,6-dihydropyridine-1(2H)-carboxamide

The trifluoroacetic acid salt of the title compound was prepared using the procedure in Example 11 replacing (2-isocyanatoethyl)benzene with 2-(2-isocyanatoethyl)thiophene. ¹H NMR (300 MHz, DMSO-d₆/Deuterium Oxide, Temp=120 C) δ ppm 8.20-8.22 (m, 2H), 7.44 (s, 1H), 7.22 (dd, J=5.1, 1.2 Hz, 1H), 7.09-7.13 (m, 1H), 6.92 (dd, J=5.1, 3.4 Hz, 1H), 6.85-6.87 (m, 1H), 6.11-6.14 (m, 1H), 4.01 (q, J=2.7 Hz, 2H), 3.56 (t, J=5.7 Hz, 2H), 3.36 (t, J=7.2 Hz, 2H), 2.97-3.03 (m, 2H), 2.48-2.55 (m, 2H); MS (APCI+) M/Z 353 (M+H)⁺.

### Example 48

### N-(2-furylmethyl)-4-(1H-pyrrolo[2,3-b]pyridin-3-yl)-3 6-dihydropyridine-1(2H)-carboxamide

The title compound was prepared using the procedure in Example 11 replacing (2-isocyanatoethyl)benzene with 2-(isocyanatomethyl)furan. ¹H NMR (500 MHz, DMSO-d₆) δ ppm 11.66 (bs, 1H), 8.24 (dd, J=8.0, 1.4 Hz, 1H), 8.20-8.26 (m, 2H), 7.55 (d, J=2.6 Hz, 1H), 7.53 (dd, J=0.8, 1.8 Hz, 1H), 7.09 (dd, J=4.7, 7.9 Hz, 1H), 7.00 (t, J=5.4 Hz, 1H), 6.36 (dd, J=1.9, 3.1 Hz, 1H), 6.14-6.19 (m, 2H), 4.25 (d, J=5.5 Hz, 2H), 4.00-4.06 (m, 2H), 3.57 (t, J=5.5 Hz, 2H); MS (ESI⁺) M/Z 322.9 (M+H)⁺.

### Example 49

### N-(3-phenylpropyl)-4-(1H-pyrrolo[2,3-b]pyridin-3-yl)-3 6-dihydropyridine-1(2H)-carboxamide

The title compound was prepared using the procedure in Example 11 replacing (2-isocyanatoethyl)benzene with (3-isocyanatopropyl)benzene. ¹H NMR (500 MHz, DMSO-d₆) δ ppm 11.66 (bs, 1H), 8.20-8.26 (m, 2H), 7.54 (d, J=2.7 Hz, 1H), 7.12-7.30 (m, 5H), 7.09 (dd, J=4.8, 7.8 Hz, 1H), 6.50 (t, J=5.2 Hz, 1H), 6.17 (m, 1H), 3.98-4.02 (m, 2H), 3.54 (t, J=5.9 Hz, 2H), 3.08 (q, J=7.0 Hz, 2H), 2.58 (t, J=7.9 Hz, 2H), 1.68-1.79 (m, 2H); MS (ESI⁺) M/Z 361.2 (M+H)⁺.

### Example 50

### N-(pyridin-3-ylmethyl)-4-(1H-pyrrolo[2,3-b]pyridin-3-yl)-3,6-dihydropyridine-1(2H)-carboxamide

A mixture of pyridin-3-ylmethanamine (0.023 mL, 0.23 mmol) and bis(2,5-dioxopyrrolidin-1-yl) carbonate (60 mg, 0.23 mmol) in N,N-dimethylformamide (0.8 mL) was stirred for 30 min at room temperature, and triethylamine (0.053 mL, 0.38 mmol) and 3-(1,2,3,6-tetrahydropyridin-4-yl)-1H-pyrrolo[2,3-b]pyridine (38 mg, 0.19 mmol) were added. The mixture was stirred for 3 h at room temperature, diluted with ethyl acetate, washed with water and brine, dried (Na₂SO₄), filtered, concentrated, and chromatographed (7% methanol/dichloromethane) to give the title compound as a white solid (35 mg, 0.10 mmol). ¹H NMR (300 MHz, DMSO) δ 11.69 (s, 1H), 8.50 (d, J=1.6 Hz, 1H), 8.42 (dd, J=1.7, 4.8 Hz, 1H), 8.23 (dt, J=1.4, 4.6, 9.3 Hz, 2H), 7.68 (dt, J=2.3, 7.8 Hz, 1H), 7.56 (d, J=2.5 Hz, 1H), 7.33 (ddd, J=1.0, 4.8, 7.8 Hz, 1H), 7.19 (t, J=5.7 Hz, 1H), 7.09 (dd, J=4.7, 7.9 Hz, 1H), 6.16-6.22 (m, 1H), 4.29 (d, J=5.7 Hz, 2H), 4.02-4.09 (m, 2H), 3.58 (t, J=5.6 Hz, 2H); MS (ESI⁺) M/Z 333.9 (M+H)⁺.

### Example 51

### 3-(1-{4-methyl-5-[3-(trifluoromethyl)phenyl]-1,3-oxazol-2-yl}-1,2,3,6-tetrahydropyridin-4-yl)-1H-pyrrolo[2,3-b]pyridine

### Example 51A

### 4-methyl-5-(3-(trifluoromethyl)phenyl)oxazole

A mixture of 1-(1-isocyanoethylsulfonyl)-4-methylbenzene (2.40 g, 11.5 mmol) 3-(trifluoromethyl)benzaldehyde (1.53 mL, 11.5 mmol) potassium carbonate (1.91 g, 13.8 mmol) in methanol (57 mL) was refluxed overnight, cooled to room temperature, concentrated, diluted with ethyl acetate, washed with water and brine, dried (Na₂SO₄), filtered, and concentrated to give the title compound.

### Example 51B

### 2-chloro-4-methyl-5-(3-(trifluoromethyl)phenyl)oxazole

A solution of LiHMDS in tetrahydrofuran (1M, 11.8 mL, 11.8 mmol) was added to a solution of the product from Example 51A (2.43 g, 10.7 mmol) in tetrahydrofuran (36 mL) at −78° C. The mixture was stirred for 30 min, and perchloroethane (5.06 g, 21.4 mmol) was added in one portion. The mixture was stirred and allowed to warm to room temperature overnight, concentrated, diluted with ethyl acetate, washed with water and brine, dried (Na₂SO₄), filtered, concentrated, and chromatographed (0-35% ethyl acetate/hexanes) to give the title compound (2.43 g, 10.7 mmol).

### Example 51C

### 3-(1-{4-methyl-5-[3-(trifluoromethyl)phenyl]-1,3-oxazol-2-yl}-1,2,3,6-tetrahydropyridin-4-yl)-1H-pyrrolo[2,3-b]pyridine

A mixture of 3-(1,2,3,6-tetrahydropyridin-4-yl)-1H-pyrrolo[2,3-b]pyridine (43 mg, 0.22 mmol) and the product of Example 51B (56 mg, 0.021 mmol) in n-butanol (0.6 mL) with a catalytic amount of 1N HCl was heated at 80 C overnight, cooled to room temperature, diluted with ethyl acetate, washed with water and brine, dried (Na₂SO₄), filtered, concentrated, and chromatographed (ethyl acetate) to give the title compound as a dark red solid (16 mg, 0.038 mmol). ¹H NMR (300 MHz, CD₃OD) δ 8.33 (dd, J=1.5, 8.0 Hz, 1H), 8.20 (dd, J=1.4, 4.8 Hz, 1H), 7.84-7.68 (m, 2H), 7.61 (t, J=8.0 Hz, 1H), 7.54-7.46 (m, 2H), 7.16 (dd, J=4.8, 8.0 Hz, 1H), 6.30 (sept, J=1.4 Hz, 1H), 4.30 (dd, J=2.4, 3.0 Hz, 2H), 3.88 (t, J=5.8 Hz, 2H), 2.75 (m, 2H), 2.36 (s, 3H); MS (ESI⁺) M/Z 425.0 (M+H)⁺.

### Example 52

### N-(2,3-dihydro-1,4-benzodioxin-5-ylmethyl)-4-(1H-pyrrolo[2,3-b]pyridin-3-yl)-3,6-dihydropyridine-1(2H)-carboxamide

The title compound was prepared using the procedure in Example 50 replacing pyridin-3-ylmethanamine with (2,3-dihydrobenzo[b][1,4]dioxin-5-yl)methanamine hydrochloride. ¹H NMR (300 MHz, DMSO-d₆) δ ppm 11.70 (bs, 1H), 8.20-8.27 (m, 2H), 7.57 (d, J=2.5 Hz, 1H), 7.10 (dd, J=4.8, 7.9 Hz, 1H), 6.92 (t, J=5.7 Hz, 1H), 6.68-6.77 (m, 3H), 6.19 (bs, 1H), 4.20-4.30 (m, 6H), 4.05-4.09 (m, 2H), 3.60 (t, J=5.5 Hz, 2H), 2.53 (buried); MS (ESI⁺) M/Z 391.0 (M+H)⁺.

### Example 53

### N-methyl-N-[(1R)-1-phenylethyl]-4-(1H-pyrrolo[2,3-b]pyridin-3-yl)-3,6-dihydropyridine-1(2H)-carboxamide

The title compound was prepared using the procedure in Example 1B replacing the product from Example 1A with (R)-N-methyl-1-phenylethanamine. ¹H NMR (300 MHz, CD₃OD) δ 8.30 (dd, J=1.6, 8.1 Hz, 1H), 8.19 (dd, J=1.2, 4.8 Hz, 1H), 7.33-7.45 (m, 5H), 7.22-7.29 (m, 1H), 7.15 (dd, J=4.8, 8.1 Hz, 1H), 6.22 (bs, 1H), 5.23 (q, J=6.7 Hz, 2H), 4.02 (quin, J=2.6 Hz, 2H), 3.52 (m, 2H), 2.62-2.69 (m, 5H), 1.60 (d, J=6.9 Hz, 3H); MS (ESI⁺) M/Z 361.0 (M+H)⁺.

### Example 54

### benzyl 4-(1H-pyrrolo[2,3-b]pyridin-3-yl)-3,6-dihydropyridine-1(2H)-carboxylate

A mixture of 3-(1,2,3,6-tetrahydropyridin-4-yl)-1H-pyrrolo[2,3-b]pyridine (50 mg, 0.25 mmol), triethylamine (0.044 mL, 0.32 mmol), and benzyl carbonochloridate (39 μL, 0.28 mmol) in dichloromethane (0.75 mL) was stirred for 90 min at room temperature, diluted with ethyl acetate, washed with water and brine, dried (Na₂SO₄), filtered, concentrated, and chromatographed (50% ethyl acetate/dichloromethane) to give the title compound as a white solid (24 mg, 0.072 mmol). ¹H NMR (300 MHz, DMSO-d₆) δ ppm 11.68 (bs, 1H), 8.20-8.26 (m, 2H), 7.55 (d, J=2.4 Hz, 1H), 7.29-7.41 (m, 5H), 7.09 (dd, J=4.7, 7.8 Hz, 1H), 6.18 (bs, 1H), 5.13 (s, 2H), 4.13 (bs, 2H), 3.65 (bs, 2H), 2.53 (buried); MS (ESI⁺) M/Z 334.0 (M+H)⁺.

### Example 55

### 2-chlorobenzyl 4-(1H-pyrrolo[2,3-b]pyridin-3-yl)-3,6-dihydropyridine-1(2H)-carboxylate

The title compound was prepared using the procedure in Example 54 replacing benzyl carbonochloridate with 2-chlorobenzyl carbonochloridate. ¹H NMR (300 MHz, DMSO-d₆) δ ppm 11.69 (bs, 1H), 8.20-8.26 (m, 2H), 7.47-7.57 (m, 3H), 7.35-7.41 (m, 2H), 7.09 (dd, J=5.1, 8.1 Hz, 1H), 6.19 (bs, 1H), 5.20 (s, 2H), 4.14 (bs, 2H), 3.66 (bs, 2H), 2.52-2.58 (m, 2H); MS (ESI⁺) M/Z 368.0(M+H)⁺.

### Example 56

### N-[1-(2-chlorophenyl)ethyl]-4-(1H-pyrrolo[2,3-b]pyridin-3-yl)-3,6-dihydropyridine-1(2H)-carboxamide

The title compound was prepared using the procedure in Example 50 replacing pyridin-3-ylmethanamine with 1-(2-chlorophenyl)ethanamine hydrochloride. ¹H NMR (300 MHz, DMSO-d₆) δ ppm 11.67 (bs, 1H), 8.21-8.26 (m, 2H), 7.55 (d, J=2.5 Hz, 1H), 7.51 (dd, J=1.8, 7.7 Hz, 1H), 7.36 (dd, J=1.4, 7.8 Hz, 1H), 7.31 (td, J=1.8, 7.7 Hz, 1H), 7.10 (dd, J=4.8, 7.8, 1H), 6.95 (d, J=7.6, 1H), 6.18 (bs, 1H), 5.19 (quin, J=7.1 Hz, 1H), 4.08 (bs, 2H), 3.59 (t, J=5.7 Hz, 2H), 1.35 (d, J=7.1 Hz, 3H); MS (ESI⁺) M/Z 381.0(M+H)⁺.

### Example 57

### 3-{1-[(4S)-4-phenyl-4,5-dihydro-1,3-oxazol-2-yl]-1,2,3,6-tetrahydropyridin-4-yl}-1H-pyrrolo[2,3-b]pyridine

A mixture of the product of Example 1C (40.8 mg, 0.113 mmol), iodine (3.1 mg, 0.12 mmol), triethylamine (0.050 mL, 0.36 mmol), and triphenylphosphine (34.7 mg, 0.132 mmol) in dichloromethane (0.6 mL) was stirred overnight at room temperature, heated to 50 C for 2 h, diluted with ethyl acetate, washed with 25% sat Na₂SO₃ and brine, dried (Na₂SO₄), filtered, concentrated, and chromatographed (10% methanol/dichloromethane) to give the title compound as a tan solid (29 mg, 0.084 mmol). ¹H NMR (300 MHz, DMSO-d₆) δ ppm 11.69 (bs, 1H), 8.21-8.28 (m, 2H), 7.57 (d, J=2.4 Hz, 1H), 7.20-7.35 (m, 5H), 7.10 (dd, J=4.8, 7.8, 1H), 6.23 (bs, 1H), 5.06 (dd, J=7.5, 9.2 Hz, 1H), 4.69 (dd, J=8.1, 9.1 Hz, 1H), 4.07-4.12 (m, 2H), 3.98 (t, J=7.8 Hz, 1H), 3.62 (t, J=5.8, 2H), 2.58 (bs, 2H); MS (ESI⁺) M/Z 345.0(M+H)⁺.

### Example 58

### N-[3-fluoro-5-(trifluoromethyl)benzyl]-4-(1H-pyrrolo[2,3-b]pyridin-3-yl)-3,6-dihydropyridine-1(2H)-carboxamide

A solution of 4-nitrophenyl carbonochloridate (34 mg, 0.17 mmol) in tetrahydrofuran (1.0 mL) was added to a solution of (3-fluoro-5-(trifluoromethyl)phenyl)methanamine (32 mg, 0.17 mmol) and triethylamine (0.043 mL, 0.31 mmol) in tetrahydrofuran (1.0 mL), and stirred for 30 min at room temperature. A mixture of 3-(1,2,3,6-tetrahydropyridin-4-yl)-1H-pyrrolo[2,3-b]pyridine (28 mg, 0.14 mmol) in tetrahydrofuran (1.0 mL) was added and heated to 50 C for 4 hours. The mixture was passed through a SiliCycle SiliaBond Carbonate solid phase extraction column with methanol, concentrated, and purified by preparative HPLC on a Phenomenex Luna C8(2) 5 um 100 Å AXIA column (30 mm×75 mm) using a gradient of acetonitrile (A) and 0.1% trifluoroacetic acid in water (B), at a flow rate of 50 mL/min (0-0.5 min 10% A, 0.5-6.0 min linear gradient 10-100% A, 6.0-7.0 min 100% A, 7.0-8.0 min linear gradient 100-10% A) to provide the trifluoroacetic acid salt of the title compound. ¹H NMR (500 MHz, DMSO-d₆/Deuterium Oxide) δ ppm 8.27 (dd, J=8.0, 1.5 Hz, 1H), 8.24 (dd, J=4.7, 1.5 Hz, 1H), 7.55 (s, 1H), 7.48-7.51 (m, 2H), 7.41-7.44 (m, 1H), 7.14 (dd, J=8.0, 4.7 Hz, 1H), 6.20-6.22 (m, 1H), 4.35-4.36 (bs, 2H), 4.07 (d, J=3.0 Hz, 2H), 3.60 (t, J=5.7 Hz, 2H), 2.51-2.56 (m, 2H) ; MS (ESI⁻) M/Z 417 (M−H)⁻.

### Example 59

### 4-(1H-pyrrolo[2,3-b]pyridin-3-yl)-N-{4-[(trifluoromethyl)thio]benzyl}-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (4-(trifluoromethylthio)phenyl)methanamine. ¹H NMR (500 MHz, DMSO-d₆/Deuterium Oxide) δ ppm 8.27 (dd, J=8.0, 1.5 Hz, 1H), 8.24 (dd, J=4.7, 1.5 Hz, 1H), 7.65-7.67 (m, 2H), 7.55 (s, 1H), 7.43-7.45 (m, 2H), 7.14 (dd, J=8.0, 4.7 Hz, 1H), 6.20-6.22 (m, 1H), 4.34 (d, J=5.3 Hz, 2H), 4.06-4.08 (m, 2H), 3.60 (t, J=5.6 Hz, 2H), 2.50-2.55 (m, 2H); MS (ESI⁻) M/Z 431 (M−H)⁻.

### Example 60

### 4-(1H-pyrrolo[2,3-b]pyridin-3-yl)-N-[4-(trifluoromethoxy)benzyl]-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (4-(trifluoromethoxy)phenyl)methanamine. ¹H NMR (500 MHz, DMSO-d₆/Deuterium Oxide) δ ppm 8.27 (dd, J=8.0, 1.5 Hz, 1H), 8.24 (dd, J=4.7, 1.5 Hz, 1H), 7.55 (s, 1H), 7.37-7.42 (m, 2H), 7.28-7.30 (m, 2H), 7.14 (dd, J=8.0, 4.7 Hz, 1H), 6.19-6.21 (m, 1H), 4.30 (d, J=5.3 Hz, 2H), 4.05-4.07 (m, 2H), 3.59 (t, J=5.7 Hz, 2H), 2.51-2.56 (m, 2H); MS (ESI⁻) M/Z 415 (M−H)⁻.

### Example 61

### 4-(1H-pyrrolo[2,3-b]pyridin-3-yl)-N-[3-(trifluoromethoxy)benzyl]-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (3-(trifluoromethoxy)phenyl)methanamine. ¹H NMR (500 MHz, DMSO-d₆/Deuterium Oxide) δ ppm 8.27 (dd, J=8.0, 1.5 Hz, 1H), 8.24 (dd, J=4.7, 1.5 Hz, 1H), 7.55 (s, 1H), 7.45 (t, J=7.9 Hz, 1H), 7.32 (d, J=7.8 Hz, 1H), 7.23-7.28 (m, 2H), 7.20 (d, J=7.4 Hz, 1H), 7.14 (dd, J=8.0, 4.7 Hz, 1H), 6.19-6.22 (m, 1H), 4.32 (d, J=5.4 Hz, 2H), 4.06-4.08 (m, 2H), 3.60 (t, J=5.6 Hz, 2H), 2.50-2.55 (m, 2H); MS (ESI⁻) M/Z 415 (M−H)⁻.

### Example 62

### N-(2,3-dimethoxybenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (2,3-dimethoxyphenyl)methanamine. ¹H NMR (500 MHz, DMSO-d₆/Deuterium Oxide) δ ppm 8.27 (dd, J=8.0, 1.5 Hz, 1H), 8.24 (dd, J=4.7, 1.5 Hz, 1H), 7.55 (s, 1H), 7.14 (dd, J=8.0, 4.7 Hz, 1H), 7.01 (t, J=7.9 Hz, 1H), 6.97 (t, J=5.8 Hz, 1H), 6.92 (dd, J=8.2, 1.5 Hz, 1H), 6.83 (dd, J=7.7, 1.5 Hz, 1H), 6.20-6.21 (m, 1H), 4.30 (d, J=5.3 Hz, 2H), 4.06-4.08 (m, 2H), 3.79 (s, 3H), 3.75 (s, 3H), 3.60 (t, J=5.6 Hz, 2H), 2.50-2.56 (m, 2H); MS (ESI⁻) M/Z 391 (M−H)⁻.

### Example 63

### N-(2,5-difluorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (2,5-difluorophenyl)methanamine. ¹H NMR (500 MHz, DMSO-d₆/Deuterium Oxide) δ ppm 8.27 (dd, J=8.0, 1.5 Hz, 1H), 8.24 (dd, J=4.7, 1.4 Hz, 1H), 7.55 (s, 1H), 7.06-7.23 (m, 5H), 6.20-6.22 (m, 1H), 4.31 (d, J=5.3 Hz, 2H), 4.07-4.08 (m, 2H), 3.60 (t, J=5.7 Hz, 2H), 2.50-2.58 (m, 2H); MS (ESI⁺) M/Z 369 (M+H)⁺.

### Example 64

### 4-(1H-pyrrolo[2,3-b]pyridin-3-yl)-N-1,2,3,4-tetrahydronaphthalen-1-yl-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with 1,2,3,4-tetrahydronaphthalen-1-amine. ¹H NMR (500 MHz, DMSO-d₆/Deuterium Oxide) δ ppm 8.26 (dd, J=8.0, 1.5 Hz, 1H), 8.23 (dd, J=4.7, 1.5 Hz, 1H), 7.55 (s, 1H), 7.19-7.24 (m, 1H), 7.12-7.15 (m, 3H), 7.07-7.11 (m, 1H), 6.76 (d, J=8.6 Hz, 1H), 6.19-6.21 (m, 1H), 4.91-4.93 (m, 1H), 4.02-4.12 (m, 2H), 3.62-3.62 (bs, 2H), 2.70-2.75 (m, 2H), 2.49-2.56 (m, 2H), 1.86-1.99 (m, 2H), 1.65-1.79 (m, 2H); MS (ESI⁻) M/Z 371 (M−H)⁻.

### Example 65

### N-(2,6-difluorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (2,6-difluorophenyl)methanamine. ¹H NMR (500 MHz, DMSO-d₆/Deuterium Oxide) δ ppm 8.25 (dd, J=8.0, 1.6 Hz, 1H), 8.23 (dd, J=4.8, 1.6 Hz, 1H), 7.53 (s, 1H), 7.33-7.39 (m, 1H), 7.13 (dd, J=7.9, 4.7 Hz, 1H), 6.99-7.09 (m, 2H), 6.93 (t, J=5.2 Hz, 1H), 6.17 (d, J=3.4 Hz, 1H), 4.33 (d, J=4.6 Hz, 2H), 3.99-4.01 (m, 2H), 3.54 (t, J=5.7 Hz, 2H), 2.46-2.49 (m, 2H); MS (ESI⁻) M/Z 367 (M−H)⁻.

### Example 66

### N-(1,2-diphenylethyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with 1,2-diphenylethanamine. ¹H NMR (500 MHz, DMSO-d₆/Deuterium Oxide) δ ppm 8.22-8.25 (m, 2H), 7.51 (s, 1H), 7.37-7.39 (m, 2H), 7.28-7.32 (m, 2H), 7.22-7.28 (m, 4H), 7.18-7.22 (m, 1H), 7.12-7.17 (m, 2H), 6.94 (d, J=8.4 Hz, 1H), 6.14-6.16 (m, 1H), 4.92-4.97 (m, 1H), 4.02-4.07 (m, 1H), 3.91-3.96 (m, 1H), 3.45-3.59 (m, 2H), 3.07 (dd, J=13.6, 9.7 Hz, 1H), 2.97 (dd, J=13.6, 5.9 Hz, 1H), 2.38-2.47 (m, 2H); MS (ESI⁻) M/Z 421 (M−H)⁻.

### Example 67

### N-(2,4-difluorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (2,4-difluorophenyl)methanamine. ¹H NMR (500 MHz, DMSO-d₆/Deuterium Oxide) δ ppm 8.26 (dd, J=8.0, 1.5 Hz, 1H), 8.23 (dd, J=4.7, 1.5 Hz, 1H), 7.55 (s, 1H), 7.36-7.41 (m, 1H), 7.12-7.17 (m, 3H), 6.98-7.06 (m, 1H), 6.18-6.21 (m, 1H), 4.29 (d, J=5.2 Hz, 2H), 4.05-4.06 (m, 2H), 3.59 (t, J=5.6 Hz, 2H), 2.50-2.55 (m, 2H); MS (ESI⁻) M/Z 367 (M−H)⁻.

### Example 68

### N-(2,5-dimethoxybenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (2,5-dimethoxyphenyl)methanamine. ¹H NMR (500 MHz, DMSO-d₆/Deuterium Oxide) δ ppm 8.27 (dd, J=8.0, 1.5 Hz, 1H), 8.24 (dd, J=4.7, 1.5 Hz, 1H), 7.55 (s, 1H), 7.14 (dd, J=8.0, 4.7 Hz, 1H), 6.94 (t, J=5.8 Hz, 1H), 6.88 (d, J=8.7 Hz, 1H), 6.73-6.77 (m, 2H), 6.21-6.23 (m, 1H), 4.23 (d, J=5.2 Hz, 2H), 4.08-4.10 (m, 2H), 3.75 (s, 3H), 3.63 (s, 3H), 3.61 (t, J=5.7 Hz, 2H), 2.51-2.56 (m, 2H); MS (ESI⁺) M/Z 393 (M+H)⁺.

### Example 69

### N-(2,3-dichlorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (2,3-dichlorophenyl)methanamine. ¹H NMR (500 MHz, DMSO-d₆/Deuterium Oxide) δ ppm 8.28 (dd, J=8.0, 1.5 Hz, 1H), 8.24 (dd, J=4.7, 1.4 Hz, 1H), 7.56 (s, 1H), 7.52 (dd, J=7.8, 1.7 Hz, 1H), 7.34 (t, J=7.8 Hz, 1H), 7.30 (dd, J=7.8, 1.7 Hz, 1H), 7.14 (dd, J=8.0, 4.7 Hz, 1H), 6.21-6.22 (m, 1H), 4.37-4.38 (bs, 2H), 4.09-4.10 (m, 2H), 3.62 (t, J=5.7 Hz, 2H), 2.51-2.56 (m, 2H); MS (APCI+) M/Z 401 (M+H)⁺.

### Example 70

### N-(3 5-dichlorobenzyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with (3,5-dichlorophenyl)methanamine. ¹H NMR (500 MHz, DMSO-d₆/Deuterium Oxide) δ ppm 8.27 (dd, J=8.0, 1.5 Hz, 1H), 8.23 (dd, J=4.7, 1.5 Hz, 1H), 7.55 (s, 1H), 7.43 (t, J=1.9 Hz, 1H), 7.30-7.31 (m, 2H), 7.14 (dd, J=8.0, 4.7 Hz, 1H), 6.20 (d, J=3.5 Hz, 1H), 4.26-4.28 (m, 2H), 4.06-4.07 (m, 2H), 3.59 (t, J=5.7 Hz, 2H), 2.50-2.55 (m, 2H); MS (ESI⁻) M/Z 399 (M−H)⁻.

### Example 71

### N-(2-cyclohex-1-en-1-ylethyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with 2-cyclohexenylethanamine. ¹H NMR (500 MHz, DMSO-d₆/Deuterium Oxide) δ ppm 8.25 (dd, J=8.0, 1.5 Hz, 1H), 8.23 (dd, J=4.7, 1.5 Hz, 1H), 7.53 (s, 1H), 7.14 (dd, J=8.0, 4.7 Hz, 1H), 6.44 (t, J=5.5 Hz, 1H), 6.17-6.19 (m, 1H), 5.37-5.39 (bs, 1H), 3.98-4.00 (m, 2H), 3.53 (t, J=5.6 Hz, 2H), 3.10-3.15 (m, 2H), 2.47-2.50 (m, 2H), 2.05 (t, J=7.4 Hz, 2H), 1.88-1.93 (m, 4H), 1.52-1.57 (m, 2H), 1.42-1.50 (m, 2H); MS (ESI⁻) M/Z 349 (M−H)⁻.

### Example 72

### N-(3,3-diphenylpropyl)-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with 3,3-diphenylpropan-1-amine. ¹H NMR (500 MHz, DMSO-d₆/Deuterium Oxide) δ ppm 8.25 (dd, J=8.0, 1.5 Hz, 1H), 8.23 (dd, J=4.7, 1.5 Hz, 1H), 7.53 (s, 1H), 7.26-7.32 (m, 8H), 7.12-7.18 (m, 3H), 6.17-6.18 (m, 1H), 3.96-4.00 (m, 3H), 3.52 (t, J=5.6 Hz, 2H), 2.99 (dd, J=8.6, 5.7 Hz, 2H), 2.46-2.50 (m, 2H), 2.19-2.24 (m, 2H) ; MS (ESI⁻) M/Z 435 (M−H)⁻.

### Example 73

### N-[2-(1H-indol-3-yl)ethyl]-4-(1H-pyrrolo[2,3-b]pyridin-3-yl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with 2-(1H-indol-3-yl)ethanamine. ¹H NMR (500 MHz, DMSO-d₆/Deuterium Oxide) δ ppm 8.26 (dd, J=8.0, 1.5 Hz, 1H), 8.24 (dd, J=4.7, 1.5 Hz, 1H), 7.58 (d, J=7.9 Hz, 1H), 7.54 (s, 1H), 7.35 (d, J=8.1 Hz, 1H), 7.13-7.16 (m, 2H), 7.06-7.09 (m, 1H), 6.97-7.01 (m, 1H), 6.18-6.20 (m, 1H), 4.01-4.02 (m, 2H), 3.57 (t, J=5.6 Hz, 2H), 3.32-3.36 (m, 2H), 2.84-2.88 (m, 2H), 2.45-2.53 (m, 2H); MS (ESI⁺) M/Z 386 (M+H)⁺.

### Example 74

### 4-(1H-pyrrolo[2,3-b]pyridin-3-yl)-N-(thien-2-ylmethyl)-3,6-dihydropyridine-1(2H)-carboxamide

The trifluoroacetic acid salt of the title compound was prepared using the procedure in Example 58 replacing (3-fluoro-5-(trifluoromethyl)phenyl)methanamine with thiophen-2-ylmethanamine. ¹H NMR (500 MHz, DMSO-d₆/Deuterium Oxide) δ ppm 8.26 (dd, J=8.0, 1.5 Hz, 1H), 8.23 (dd, J=4.7, 1.5 Hz, 1H), 7.54 (s, 1H), 7.32 (dd, J=4.9, 1.4 Hz, 1H), 7.14 (dd, J=8.0, 4.7 Hz, 1H), 6.93-6.97 (m, 2H), 6.19 (d, J=3.4 Hz, 1H), 4.43 (s, 2H), 4.03-4.04 (m, 2H), 3.58 (t, J=5.6 Hz, 2H), 2.48-2.54 (m, 2H); MS (ESI⁺) M/Z 339 (M+H)⁺.

### Example 75

### 3-[1-(3-pyridin-3-yl-1,2,4-oxadiazol-5-yl)-1,2,3,6-tetrahydropyridin-4-yl]-1 H-pyrrolo[2,3-b]pyridine

A solution of 3-(pyridin-3-yl)-5-(trichloromethyl)-1,2,4-oxadiazole (200 mg, 0.757 mmol) and 3-(1,2,3,6-tetrahydropyridin-4-yl)-1H-pyrrolo[2,3-b]pyridine (197 mg, 0.987 mmol) in DMSO (1 mL) was stirred at room temperature for 5 h. The mixture was diluted with methanol (3 mL), filtered, and washed with additional methanol (5×1 mL) to afford a white solid. ¹H NMR (300 MHz, CDCl₃) 2.73-2.78 (m, 2H), 4.01 (t, J=5.8 Hz, 2H), 4.43 (q, J=2.5 Hz, 2H), 6.23-6.26 (m, 1H), 7.05-7.14 (m, 4H), 7.16 (dd, J=8.1, 4.8 Hz, 1H), 7.33 (d, J=2.7 Hz, 1H), 7.39 (ddd, J=7.9, 4.8, 0.9 Hz, 1H), 8.19 (dd, J=8.0, 0.9 Hz, 1H), 8.29 (dt, J=8.1, 2.0 Hz, 1H), 8.35 (dd, J=4.6, 1.5 Hz, 1H), 8.71 (dd, J=4.8, 1.7 Hz, 1H), 8.79 (br s, 1H), 9.25 (dd, J=2.2, 0.9 Hz, 1H) ; MS (DCI⁺) M/Z 345.2 (M+H)⁺.

### Example 76

### N-[(1R)-1-(3-methoxyphenyl)ethyl]-4-(1H-pyrrolo[2,3-b]pyridin-3-yl)-3,6-dihydropyridine-1(2H)-carboxamide

The title compound was prepared using the procedure in Example 1B replacing the product from Example 1A with (R)-1-(3-methoxyphenyl)ethanamine. ¹H NMR (300 MHz, DMSO-d₆) δ ppm 11.66 (bs, 1H), 8.20-8.26 (m, 2H), 7.54 (d, J=2.4 Hz, 1H), 7.20 (t, J=8.1 Hz, 1H), 7.09 (dd, J=5.1, 7.8 Hz, 1H), 6.88-6.93 (m, 2H), 6.72-6.79 (m, 2H), 6.17 (bs, 1H), 4.84 (quin, J=7.5 Hz, 1H), 4.03-4.09 (m, 2H), 3.72 (s, 3H), 3.58 (t, J=5.4 Hz, 2H), 1.37 (d, J=7.1 Hz, 3H); MS (ESI⁺) M/Z 377.0 (M+H)⁺.

### Example 77

### N-[(1R)-1-(3-methoxyphenyl)ethyl]-4-(1H-pyrrolo[2,3-b]pyridin-4-yl)-3,6-dihydropyridine-1(2H)-carboxamide

### Example 77A

### tert-butyl 4-(1H-pyrrolo[2,3-b]pyridin-4-yl)-5,6-dihydropyridine-1(2H)-carboxylate

A mixture of 4-bromo-1H-pyrrolo[2,3-b]pyridine (102 mg, 0.520 mmol), tert-butyl 4-(4,4,5,5-tetramethyl-1,3,2-dioxaborolan-2-yl)-5,6-dihydropyridine-1(2H)-carboxylate (161 mg, 0.519 mmol), potassium phosphate (224 mg, 1.05 mmol), and dichlorobis(triphenylphosphine)palladium(II) (18 mg, 0.025 mmol) in 1,2-dimethoxyethane (2 mL) and water (1 mL) was irradiated in a microwave to 140 OC for 20 min, cooled to room temperature, diluted with ethyl acetate, washed with water and brine, dried (Na₂SO₄), filtered, concentrated, and chromatographed (2% methanol/dichloromethane) and triturated (diethyl ether/hexanes) to give the title compound as a white solid (116 mg, 0.387 mmol).

### Example 77B

### N-[(1R)-1-(3-methoxyphenyl)ethyl]-4-(1H-pyrrolo[2,3-b]pyridin-4-yl)-3 6-dihydropyridine-1(2H)-carboxamide

### Step A

The product of Example 77A was stirred in 10% CF₃CO₂H in methanol (3 mL) for 1 h at room temperature, concentrated, diluted with sat NaHCO₃, extracted with dichloromethane, and dried (Na₂SO₄). The aqueous and organic layers were combined, concentrated, triturated with 20% isopropanol/CHCl₃, and concentrated to give 280 mg of a tan gum.

### Step B

A mixture of (R)-1-(3-methoxyphenyl)ethanamine (71.5 mg, 0.473 mmol), triethylamine (0.081 mL, 0.58 mmol), and triphosgene (48.5 mg, 0.163 mmol) in dichloromethane (1.5 mL) was stirred 2 h at room temperature, and added to a mixture of the product from Step A, triethylamine (0.08 mL, 0.6 mmol), and N,N-dimethylformamide (2 mL). The resulting mixture was stirred overnight at room temperature, diluted with ethyl acetate, washed with water and brine, dried (Na₂SO₄), filtered, concentrated, and chromatographed (25% acetone/dichloromethane) to give 36 mg the title compound as a white solid (36 mg, 0.096 mmol). ¹H NMR (300 MHz, DMSO-d₆) δ ppm 11.66 (bs, 1H), 8.17 (d, J=4.8 Hz, 1H), 7.47 (t, J=2.8 Hz, 1H), 7.21 (t, J=7.9 Hz, 1H), 6.99 (d, J=5.2 Hz, 1H), 6.89-6.94 (m, 2H), 6.72-6.84 (m, 2H), 6.61 (dd, J=2.0, 3.6 Hz, 1H), 6.37 (bs, 1H), 4.85 (quin, J=7.1 Hz, 1H), 4.08-4.14 (m, 2H), 3.73 (s, 3H), 3.61 (t, J=5.6 Hz, 2H), 2.57 (bs, 2H), 1.38 (d, J=7.1 Hz, 3H); MS (ESI⁺) M/Z 377.0 (M+H)⁺.

### Example 78

### N-[(1R)-1-(3-methoxyphenyl)ethyl]-4-(1H-pyrrolo[2,3-b]pyridin-5-yl)-3,6-dihydropyridine-1(2H)-carboxamide

The title compound was prepared using the procedures described for the preparation of Example 77, replacing 4-bromo-1H-pyrrolo[2,3-b]pyridine used in Example 77A with 5-bromo-1H-pyrrolo[2,3-b]pyridine. ¹H NMR (300 MHz, DMSO-d₆) δ ppm 11.59 (bs, 1H), 8.35 (d, J=2.1 Hz, 1H), 7.97 (d, J=1.9 Hz, 1H), 7.44 (t, J=2.6 Hz, 1H), 7.21 (t, J=8.1 Hz, 1H), 6.88-6.94 (m, 2H), 6.73-6.81 (m, 2H), 6.42 (dd, J=1.9, 3.4 Hz, 1H), 6.16 (bs, 1H), 4.85 (quin, J=7.3 Hz, 1H), 4.01-4.07 (m, 2H), 3.73 (s, 3H), 3.59 (t, J=6.1 Hz, 2H), 2.53 (buried), 1.37 (d, J=7.1 Hz, 3H); MS (ESI⁺) M/Z 377.0 (M+H)⁺.

It is understood that the foregoing detailed description and accompanying examples are merely illustrative and are not to be taken as limitations upon the scope of the invention, which is defined solely by the appended claims and their equivalents. Various changes and modifications to the disclosed embodiments can be apparent to those skilled in the art. Such changes and modifications, including without limitation those relating to the chemical structures, substituents, derivatives, intermediates, syntheses, formulations and/or methods of use of the invention, can be made without departing from the spirit and scope thereof.

What we claim is:

1. A compound of formula (I) or a pharmaceutically acceptable salt, solvate, prodrug, salt of a prodrug, or a combination thereof 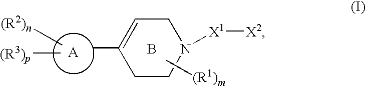
wherein
R¹ represents optional substituent(s) on ring B, and each occurrence of R¹ is independently alkyl, CN, —O(R^{1a}), —N(R^{1b})(R^{1c}), —(C₁₋₆ alkylenyl)-O(R^{1a}), —(C₁₋₆ alkylene)-N(R^{1b})(R^{1c}), —(C₁₋₆ alkylene)-CN, alkenyl, halogen, or haloalkyl;
R^{1a} and R^{1b}, at each occurrence, are each independently hydrogen, alkyl or haloalkyl;
R^{1c}, at each occurrence, is independently hydrogen, alkyl, haloalkyl, O(R^{za}), C(O)NR^{za}R^{zb}, C(O)R^{zb}, S(O)₂R^{zc}, or S(O)₂NR^{za}R^{zb}; wherein each occurrence of R^{za} and R^{zb} are each independently hydrogen, alkyl or haloalkyl, and R^{zc} is alkyl or haloalkyl;
R² represents optional substituent(s) on the carbon atom(s) of ring A, and each occurrence of R² is independently aryl, heteroaryl, cycloalkyl, cycloalkenyl, heterocycle, arylalkyl, heteroarylalkyl, cycloalkylalkyl, cycloalkenylalkyl, or heterocyclealkyl; wherein each of the aryl, heteroaryl, cycloalkyl, cycloalkenyl, and heterocycle moieties, as a substituent or part of a substituent, is independently unsubstituted or substituted with 1, 2, 3, 4, or 5 substituents as represented by R^{7a};
R³ represents optional substituent(s) on the carbon atom(s) of ring A;
m is 0, 1,2, or 3;
n is 0 or 1;
p is 0, 1, 2, or 3;
A is formula (i), (ii), or (iii) 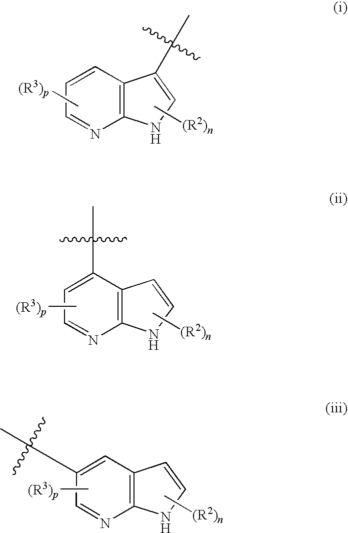
wherein 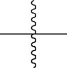
represents the point of connection to ring B; and R² and R³ are optional substituents on any substitutable carbon atoms within the bicyclic ring;
X¹ is C(O), C(S), C(O)O, C(O)N(R⁴), S(O), S(O)₂, S(O)₂N(R⁴), or C(═NR⁵); wherein the C(O)O, C(O)N(R⁴), and the S(O)₂N(R⁴) are connected to the nitrogen atom of ring B through the carbon and the sulfur atoms respectively; and
x² is hydroxyalkyl, —(CR^{6a}R^{6b})_{q}-G¹, -alkenylene-G¹, —(CR^{6a}R^{6b})ᵣ—X³-G¹, —(CR^{6a}R^{6b})_{q}—X³—(CR^{6a}R^{6b})_{q}-G¹, or J^{A} wherein
X³ is O, S, N(H), or N(alkyl);
G¹ at each occurrence, is independently cycloalkyl, cycloalkenyl, heterocycle, heteroaryl, or aryl, each of which is independently unsubstituted or substituted with 1, 2, 3, 4, or 5 substituents as represented by R^{7b};
J^{A} is a monocyclic heterocycle or a monocyclic cycloalkyl optionally substituted with 1, 2, 3, 4, 5, or 6 substituents as represented by R^{7JA}; two R^{7JA} on the adjacent carbon atoms of J^{A}, together with the carbon atoms to which they are attached, optionally form a benzo, a monocyclic heterocycle, a monocyclic cycloalkyl, or a monocyclic cycloalkenyl ring wherein each of said rings is independently unsubstituted or substituted with 1, 2, or 3 substituents as represented by R^{7b};
R^{6a} and R^{6b} can be the same or different, and at each occurrence, are each independently hydrogen, halogen, haloalkyl, aryl, —OR^{u}, —N(R^{v})(R^{w}), or alkyl; wherein the alkyl is optionally substituted with one substituent selected from the group consisting of —OR^{u}, —N(R^{v})(R^{w}), aryl, and monocyclic heterocycle; wherein the aryl group and the monocyclic heterocycle group are each independently unsubstituted or substituted with 1, 2, 3, 4, or 5 substituents as represented by R^{6za};
or
X¹-X² together is a five membered monocyclic heterocycle or a five membered monocyclic heteroaryl ring, optionally substituted with 1, 2, 3, or 4 substituents as represented by R^{7c};
R⁴ is hydrogen or alkyl which is optionally substituted with 1 or 2 substituents independently selected from the group consisting of OH, O(alkyl), halogen, —C(O)(alkyl), —C(O)O(alkyl), —C(O)NH₂, —C(O)N(H)(alkyl), —C(O)N(alkyl)₂, cycloalkyl, cycloalkenyl, heterocycle, aryl, and heteroaryl;
R^{u}, R^{v}, and R^{w}, at each occurrence, are each independently hydrogen, alkyl, or haloalkyl;
R^{7JA} and R^{7c}, at each occurrence, are each independently alkyl, alkenyl, alkynyl, halogen, oxo, NO₂, CN, haloalkyl, OR^{a}, OC(O)R^{a}, NR^{a}R^{b}, N(R^{b})C(O)R^{a}, N(R)S(O)₂R^{a}, SR^{a}, S(O)R^{c}, S(O)₂R^{c}, S(O)₂NR^{a}R^{b}, C(O)R^{a}, C(O)OR^{a}, C(O)NR^{a}R^{b}, —(C₁₋₆alkylene)-NO₂, —(C₁₋₆ alkylene)-CN, —(C₁₋₆ alkylene)-OR^{a}, —(C₁₋₆ alkylene)-OC(O)R^{a}, —(C₁₋₆ alkylene)-NR^{a}R^{b}, —(C₁₋₆ alkylene)-N(R^{b})C(O)R^{a}, —(C₁₋₆ alkylene)-N(R^{b})S(O)₂R^{a}, —(C₁₋₆ alkylene)-SR^{a}, —(C₁₋₆ alkylene)-S(O)R^{c}, —(C₁₋₆ alkylene)-S(O)₂R^{c}, —(C₁₋₆ alkylene)-S(O)₂NR^{a}R^{b}, —(C₁₋₆ alkylene)- C(O)R^{a}, —(C₁₋₆ alkylene)-C(O)OR^{a}, —(C₁₋₆ alkylene)-C(O)NR^{a}R^{b}, G², —(C₁₋₆ alkylene)-G², or —O(CR^{ax}R^{bx})ₜO— wherein the oxygen atoms of —O(CR^{ax}R^{bx})ₜO— are connected to the adjacent carbon atoms of the phenyl group;
R^{7b}, at each occurrence, is independently alkyl, alkenyl, alkynyl, halogen, oxo, NO₂, CN, haloalkyl, OR^{7ab}, OC(O)R^{7ab}, NR^{7ab}R^{b}, N(R^{b})C(O)R^{7ab}, N(R^{b})S(O)₂R^{7ab}, SR^{7ab}, S(O)R^{c}, S(O)₂R^{c}, S(O)₂NR^{7ab}R^{b}, C(O)R^{7ab}, C(O)OR^{7ab}, C(O)NR^{7ab}R^{b}, —(C₁₋₆ alkylene)-NO₂, —(C₁₋₆ alkylene)-CN, —(C₁₋₆ alkylene)-OR^{7ab}, —(C₁₋₆ alkylene)-OC(O)R^{7ab}, —(C₁₋₆ alkylene)-NR^{7ab}R^{b}, —(C₁₋₆ alkylene)-N(R^{b})C(O)R^{7ab}, —(C₁₋₆ alkylene)-N(R^{b})S(O)₂R^{7ab}, —(C₁₋₆ alkylene)-SR^{7ab}, —(C₁₋₆ alkylene)-S(O)R^{c}, —(C₁₋₆ alkylene)-S(O)₂R^{c}, —(C₁₋₆ alkylene)-S(O)₂NR^{7ab}R^{b}, —(C₁₋₆ alkylene)-C(O)R^{7ab}, —(C₁₋₆ alkylene)-C(O)OR^{7ab}, —(C₁₋₆ alkylene)-C(O)NR^{7ab}R^{b}, G², —(C₁₋₆ alkylene)-G², or —O(CR^{ax}R^{bx})ₜO— wherein the oxygen atoms of —O(CR^{ax}R^{bx})ₜO— are connected to the adjacent carbon atoms of the phenyl group;
G², at each occurrence, is independently cycloalkyl, cycloalkenyl, heterocycle, heteroaryl, or aryl, each of which is independently unsubstituted or substituted with 1, 2, 3, 4, or 5 substituents as represented by R^{7d};
R³, R^{7a}, R^{6za}, and R^{7d}, at each occurrence, are each independently alkyl, alkenyl, alkynyl, halogen, NO₂, CN, haloalkyl, OR^{a}, OC(O)R^{a}, NR^{a}R^{b}, N(R^{b})C(O)R^{a}, N(R^{b})S(O)₂R^{a}, SR^{a}, S(O)R^{c}, S(O)₂R^{c}, S(O)₂NR^{a}R^{b}, C(O)R^{a}, C(O)OR^{a}, C(O)NR^{a}R^{b}, —(C₁₋₆ alkylene)-NO₂, —(C₁₋₆ alkylene)-CN, —(C₁₋₆ alkylene)-OR^{a}, —(C₁₋₆ alkylene)-OC(O)R^{a}, —(C₁₋₆ alkylene)-NR^{a}R^{b}, —(C₁₋₆ alkylene)-N(R^{b})C(O)R^{a}, —(C₁₋₆ alkylene)-N(R^{b})S(O)₂R^{a}, —(C₁₋₆ alkylene)-SR^{a}, —(C₁₋₆ alkylene)-S(O)R^{c}, —(C₁₋₆ alkylene)-S(O)₂R^{c}, —(C₁₋₆ alkylene)-S(O)₂NR^{a}R^{b}, —(C₁₋₆ alkylene)- C(O)R^{a}, —(C₁₋₆ alkylene)-C(O)OR^{a}, or —(C₁₋₆ alkylene)-C(O)NR^{a}R^{b};
R^{a} and R^{b}, at each occurrence, are each independently hydrogen, alkyl, or haloalkyl;
R^{ax} and R^{bx}, at each occurrence, are each independently hydrogen, halogen, alkyl, or haloalkyl;
R^{7ab}, at each occurrence, is independently hydrogen, alkyl, haloalkyl, G², or —(C₁₋₆ alkylene)-G²;
R^{c}, at each occurrence, is independently alkyl or haloalkyl;
q, at each occurrence, is independently 1, 2, 3, or 4;
t is 1,2, or 3; and
r is 2, 3, or 4;
with the proviso that
(a) when A is formula (i), X¹ is C(O), and X² is -alkenylene-G¹, then G¹ is not monocyclic heteroaryl; and
(b) when A is formula (ii), X¹ is C(O), X² is —(CR^{6a}R^{6b})_{q}-G¹, and G¹ is aryl, then one of R^{6a} and R^{6b} is other than N(R^{v})(R^{w}).

2. The compound of claim 1 or a pharmaceutically acceptable salt or solvate thereof wherein X¹ is C(O), C(O)N(R⁴), C(O)O, or S(O)₂, and X² is —(CR^{6a}R^{6b})_{q}-G¹, —(CR^{6a}R^{6b})ᵣ—X³-G¹, or J^{A}.

3. The compound of claim 1 or a pharmaceutically acceptable salt or solvate thereof wherein X¹ is C(O)N(R⁴) and X² is —(CR^{6a}R^{6b})_{q}-G¹, —(CR^{6a}R^{6b})ᵣ—X³-G¹, or J^{A}.

4. The compound of claim 1 or a pharmaceutically acceptable salt or solvate thereof wherein X¹-X² together is a five membered monocyclic heterocycle or a five membered monocyclic heteroaryl ring, optionally substituted with 1, 2, 3, or 4 substituents as represented by R^{7c}.

5. The compound of claim 1 or a pharmaceutically acceptable salt or solvate thereof wherein X¹-X² together is formula (iv), (v), or (vi) 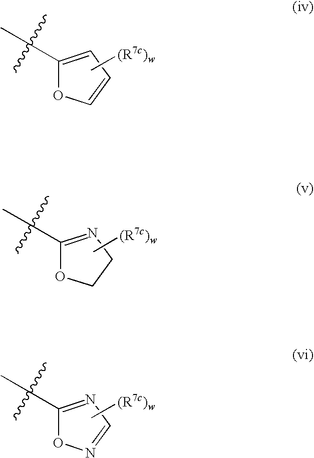
wherein w is 1 or 2.

6. The compound of claim 1 or a pharmaceutically acceptable salt or solvate thereof, having formula (Ia)

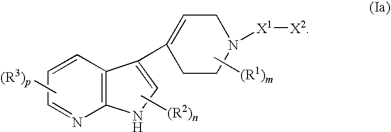

7. The compound of claim 6 or a pharmaceutically acceptable salt or solvate thereof, X¹ is C(O), C(O)N(R⁴), C(O)O, or S(O)₂, and X² is —(CR^{6a}R^{6b})_{q}-G¹, —(CR^{6a}R^{6b})ᵣ—X³-G¹, or J^{A}.

8. The compound of claim 6 or a pharmaceutically acceptable salt or solvate thereof wherein X¹ is C(O)N(R⁴) and X² is —(CR^{6a}R^{6b})_{q}-G¹, —(CR^{6a}R^{6b})ᵣ—X³-G¹, or J^{A}.

9. The compound of claim 6 or a pharmaceutically acceptable salt or solvate thereof wherein X¹-X² together is a five membered monocyclic heterocycle or a five membered monocyclic heteroaryl ring, optionally substituted with 1, 2, 3, or 4 substituents as represented by R^{7c}.

10. The compound of claim 6 or a pharmaceutically acceptable salt or solvate thereof wherein X¹-X² together is formula (iv), (v), or (vi) 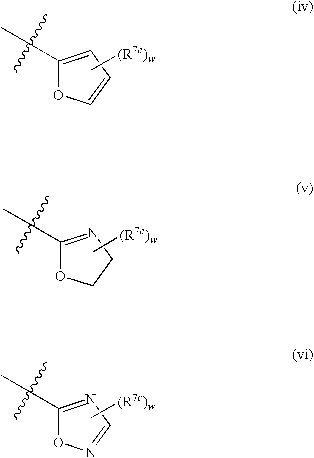
wherein w is 1 or 2.

11. The compound of claim 1 or a pharmaceutically acceptable salt or solvate thereof, having formula (Ib)

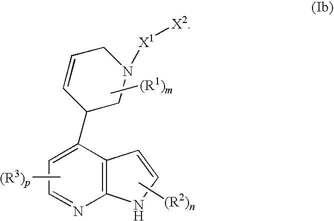

12. The compound of claim 11 or a pharmaceutically acceptable salt or solvate thereof, wherein X¹ is C(O), C(O)N(R⁴), C(O)O, or S(O)₂, and X² is —(CR^{6a}R^{6b})_{q}-G¹, —(CR^{6a}R^{6b})ᵣ—X³-G¹, or J^{A}.

13. The compound of claim 11 or a pharmaceutically acceptable salt or solvate thereof, wherein X¹-X² together is a five membered monocyclic heterocycle or a five membered monocyclic heteroaryl ring, optionally substituted with 1, 2, 3, or 4 substituents as represented by R^{7c}.

14. The compound of claim 1 or a pharmaceutically acceptable salt or solvate thereof, having formula (Ic)

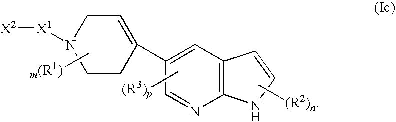

15. The compound of claim 14 or a pharmaceutically acceptable salt or solvate thereof, X¹ is C(O), C(O)N(R⁴), C(O)O, or S(O)₂, and X² is —(CR^{6a}R^{6b})_{q}-G¹, —(CR^{6a}R^{6b})ᵣ—X³-G¹, or J^{A}.

16. The compound of claim 14 or a pharmaceutically acceptable salt or solvate thereof, wherein X¹-X² together is a five membered monocyclic heterocycle or a five membered monocyclic heteroaryl ring, optionally substituted with 1, 2, 3, or 4 substituents as represented by R^{7c}.

17. The compound of claim 1 or a pharmaceutically acceptable salt or solvate thereof, selected from the group consisting of
N-[(1S)-2-hydroxy-1-phenylethyl]-4-(1H-pyrrolo[2,3-b]pyridin-3-yl)-3,6-dihydropyridine-1(2H)-carboxamide;
3-[1-(3-phenylpropanoyl)-1,2,3,6-tetrahydropyridin-4-yl]-1H-pyrrolo[2,3-b]pyridine;
3-{1-[(2-phenylethyl)sulfonyl]-1,2,3,6-tetrahydropyridin-4-yl}-1H-pyrrolo[2,3-b]pyridine;
N-benzyl-4-(1H-pyrrolo[2,3-b]pyridin-3-yl)-3,6-dihydropyridine-1(2H)-carboxamide;
N-(1-naphthylmethyl)-4-(1H-pyrrolo[2,3-b]pyridin-3-yl)-3,6-dihydropyridine-1(2H)-carboxamide;
3-{1-[(3-phenylmorpholin-4-yl)carbonyl]-1,2,3,6-tetrahydropyridin-4-yl}-1H-pyrrolo[2,3-b]pyridine;
3-{1-[(4-methyl-2-phenylpiperazin-1-yl)carbonyl]-1,2,3,6-tetrahydropyridin-4-yl}-1H-pyrrolo[2,3-b]pyridine;
N-[(1S)-1-phenylethyl]-4-(1H-pyrrolo[2,3-b]pyridin-3-yl)-3,6-dihydropyridine-1(2H)-carboxamide;
N-[(1R)-1-phenylethyl]-4-(1H-pyrrolo[2,3-b]pyridin-3-yl)-3,6-dihydropyridine-1(2H)-carboxamide;
N-(2-phenoxyethyl)-4-(1H-pyrrolo[2,3-b]pyridin-3-yl)-3,6-dihydropyridine-1(2H)-carboxamide;
N-(2-phenylethyl)-4-(1H-pyrrolo[2,3-b]pyridin-3-yl)-3,6-dihydropyridine-1(2H)-carboxamide;
N-(2,4-dichlorobenzyl)-4-(1H-pyrrolo[2,3-b]pyridin-3-yl)-3,6-dihydropyridine-1(2H)-carboxamide;
N-(2-chlorobenzyl)-4-(1H-pyrrolo[2,3-b]pyridin-3-yl)-3,6-dihydropyridine-1(2H)-carboxamide;
N-(3,4-dichlorobenzyl)-4-(1H-pyrrolo[2,3-b]pyridin-3-yl)-3,6-dihydropyridine-1(2H)-carboxamide;
N-(4-fluorobenzyl)-4-(1H-pyrrolo[2,3-b]pyridin-3-yl)-3,6-dihydropyridine-1(2H)-carboxamide;
N-(4-methoxybenzyl)-4-(1H-pyrrolo[2,3-b]pyridin-3-yl)-3,6-dihydropyridine-1(2H)-carboxamide;
N-(3-methylbenzyl)-4-(1H-pyrrolo[2,3-b]pyridin-3-yl)-3,6-dihydropyridine-1(2H)-carboxamide;
N-(4-methylbenzyl)-4-(1H-pyrrolo[2,3-b]pyridin-3-yl)-3,6-dihydropyridine-1(2H)-carboxamide;
N-(2-methylbenzyl)-4-(1H-pyrrolo[2,3-b]pyridin-3-yl)-3,6-dihydropyridine-1(2H)-carboxamide;
N-(4-bromobenzyl)-4-(1H-pyrrolo[2,3-b]pyridin-3-yl)-3,6-dihydropyridine-1(2H)-carboxamide;
N-(2-fluorobenzyl)-4-(1H-pyrrolo[2,3-b]pyridin-3-yl)-3,6-dihydropyridine-1(2H)-carboxamide;
N-(3-fluorobenzyl)-4-(1H-pyrrolo[2,3-b]pyridin-3-yl)-3,6-dihydropyridine-1(2H)-carboxamide;
4-(1H-pyrrolo[2,3-b]pyridin-3-yl)-N-(3,4,5-trimethoxybenzyl)-3,6-dihydropyridine-1(2H)-carboxamide;
N-(2-methoxybenzyl)-4-(1H-pyrrolo[2,3-b]pyridin-3-yl)-3,6-dihydropyridine-1(2H)-carboxamide;
N-(2-ethoxybenzyl)-4-(1H-pyrrolo[2,3-b]pyridin-3-yl)-3,6-dihydropyridine-1(2H)-carboxamide;
N-(3-methoxybenzyl)-4-(1H-pyrrolo[2,3-b]pyridin-3-yl)-3,6-dihydropyridine-1(2H)-carboxamide;
N-[2-(1,3-benzodioxol-5-yl)ethyl]-4-(1H-pyrrolo[2,3-b]pyridin-3-yl)-3,6-dihydropyridine-1(2H)-carboxamide;
N-[2-(3,5-dimethoxyphenyl)ethyl]-4-(1H-pyrrolo[2,3-b]pyridin-3-yl)-3,6-dihydropyridine-1(2H)-carboxamide;
N-[2-(2,3-dimethoxyphenyl)ethyl]-4-(1H-pyrrolo[2,3-b]pyridin-3-yl)-3,6-dihydropyridine-1(2H)-carboxamide;
N-[2-(3,4-dichlorophenyl)ethyl]-4-(1H-pyrrolo[2,3-b]pyridin-3-yl)-3,6-dihydropyridine-1(2H)-carboxamide;
N-[2-(2,6-dichlorophenyl)ethyl]-4-(1H-pyrrolo[2,3-b]pyridin-3-yl)-3,6-dihydropyridine-1(2H)-carboxamide;
N-[2-(5-bromo-2-methoxyphenyl)ethyl]-4-(1H-pyrrolo[2,3-b]pyridin-3-yl)-3,6-dihydropyridine-1(2H)-carboxamide;
N-[2-(3-bromo-4-methoxyphenyl)ethyl]-4-(1H-pyrrolo[2,3-b]pyridin-3-yl)-3,6-dihydropyridine-1(2H)-carboxamide;
N-[2-(2,5-dimethoxyphenyl)ethyl]-4-(1H-pyrrolo[2,3-b]pyridin-3-yl)-3,6-dihydropyridine-1(2H)-carboxamide;
N-(4-chlorobenzyl)-4-(1H-pyrrolo[2,3-b]pyridin-3-yl)-3,6-dihydropyridine-1(2H)-carboxamide;
N-[2-(2-fluorophenyl)ethyl]-4-(1H-pyrrolo[2,3-b]pyridin-3-yl)-3,6-dihydropyridine-1(2H)-carboxamide;
N-[2-(4-methoxyphenyl)ethyl]-4-(1H-pyrrolo[2,3-b]pyridin-3-yl)-3,6-dihydropyridine-1(2H)-carboxamide;
N-[2-(3-chlorophenyl)ethyl]-4-(1H-pyrrolo[2,3-b]pyridin-3-yl)-3,6-dihydropyridine-1(2H)-carboxamide;
N-[2-(2,4-dichlorophenyl)ethyl]-4-(1H-pyrrolo[2,3-b]pyridin-3-yl)-3,6-dihydropyridine-1(2H)-carboxamide;
N-[2-(4-fluorophenyl)ethyl]-4-(1H-pyrrolo[2,3-b]pyridin-3-yl)-3,6-dihydropyridine-1(2H)-carboxamide;
N-(2,2-diphenylethyl)-4-(1H-pyrrolo[2,3-b]pyridin-3-yl)-3,6-dihydropyridine-1(2H)-carboxamide;
N-[2-(3,4-dimethoxyphenyl)ethyl]-4-(1H-pyrrolo[2,3-b]pyridin-3-yl)-3,6-dihydropyridine-1(2H)-carboxamide;
N-[2-(4-chlorophenyl)ethyl]-4-(1H-pyrrolo[2,3-b]pyridin-3-yl)-3,6-dihydropyridine-1(2H)-carboxamide;
N-(cyclohexylmethyl)-4-(1H-pyrrolo[2,3-b]pyridin-3-yl)-3,6-dihydropyridine-1(2H)-carboxamide;
N-(4-phenylbutyl)-4-(1H-pyrrolo[2,3-b]pyridin-3-yl)-3,6-dihydropyridine-1(2H)-carboxamide;
N-[(1,1-dioxidotetrahydrothien-3-yl)methyl]-4-(1H-pyrrolo[2,3-b]pyridin-3-yl)-3,6-dihydropyridine-1(2H)-carboxamide;
4-(1H-pyrrolo[2,3-b]pyridin-3-yl)-N-(2-thien-2-ylethyl)-3,6-dihydropyridine-1(2H)-carboxamide;
N-(2-furylmethyl)-4-(1H-pyrrolo[2,3-b]pyridin-3-yl)-3,6-dihydropyridine-1(2H)-carboxamide;
N-(3-phenylpropyl)-4-(1H-pyrrolo[2,3-b]pyridin-3-yl)-3,6-dihydropyridine-1(2H)-carboxamide;
N-(pyridin-3-ylmethyl)-4-(1H-pyrrolo[2,3-b]pyridin-3-yl)-3,6-dihydropyridine-1(2H)-carboxamide;
3-(1-{4-methyl-5-[3-(trifluoromethyl)phenyl]-1,3-oxazol-2-yl}-1,2,3,6-tetrahydropyridin-4-yl)-1H-pyrrolo[2,3-b]pyridine;
N-(2,3-dihydro-1,4-benzodioxin-5-ylmethyl)-4-(1H-pyrrolo[2,3-b]pyridin-3-yl)-3,6-dihydropyridine-1(2H)-carboxamide;
N-methyl-N-[(1R)-1-phenylethyl]-4-(1H-pyrrolo[2,3-b]pyridin-3-yl)-3,6-dihydropyridine-1(2H)-carboxamide;
benzyl 4-(1H-pyrrolo[2,3-b]pyridin-3-yl)-3,6-dihydropyridine-1(2H)-carboxylate;
2-chlorobenzyl 4-(1H-pyrrolo[2,3-b]pyridin-3-yl)-3,6-dihydropyridine-1(2H)-carboxylate;
N-[1-(2-chlorophenyl)ethyl]-4-(1H-pyrrolo[2,3-b]pyridin-3-yl)-3,6-dihydropyridine-1(2H)-carboxamide;
3-{1-[(4S)-4-phenyl-4,5-dihydro-1,3-oxazol-2-yl]-1,2,3,6-tetrahydropyridin-4-yl}-1H-pyrrolo[2,3-b]pyridine;
N-[3-fluoro-5-(trifluoromethyl)benzyl]-4-(1H-pyrrolo[2,3-b]pyridin-3-yl)-3,6-dihydropyridine-1(2H)-carboxamide;
4-(1H-pyrrolo[2,3-b]pyridin-3-yl)-N-{4-[(trifluoromethyl)thio]benzyl}-3,6-dihydropyridine-1(2H)-carboxamide;
4-(1H-pyrrolo[2,3-b]pyridin-3-yl)-N-[4-(trifluoromethoxy)benzyl]-3,6-dihydropyridine-1(2H)-carboxamide;
4-(1H-pyrrolo[2,3-b]pyridin-3-yl)-N-[3-(trifluoromethoxy)benzyl]-3,6-dihydropyridine-1(2H)-carboxamide;
N-(2,3-dimethoxybenzyl)-4-(1H-pyrrolo[2,3-b]pyridin-3-yl)-3,6-dihydropyridine-1(2H)-carboxamide;
N-(2,5-difluorobenzyl)-4-(1H-pyrrolo[2,3-b]pyridin-3-yl)-3,6-dihydropyridine-1(2H)-carboxamide;
4-(1H-pyrrolo[2,3-b]pyridin-3-yl)-N-1,2,3,4-tetrahydronaphthalen-1-yl-3,6-dihydropyridine-1(2H)-carboxamide;
N-(2,6-difluorobenzyl)-4-(1H-pyrrolo[2,3-b]pyridin-3-yl)-3,6-dihydropyridine-1(2H)-carboxamide;
N-(1,2-diphenylethyl)-4-(1H-pyrrolo[2,3-b]pyridin-3-yl)-3,6-dihydropyridine-1(2H)-carboxamide;
N-(2,4-difluorobenzyl)-4-(1H-pyrrolo[2,3-b]pyridin-3-yl)-3,6-dihydropyridine-1(2H)-carboxamide;
N-(2,5-dimethoxybenzyl)-4-(1H-pyrrolo[2,3-b]pyridin-3-yl)-3,6-dihydropyridine-1(2H)-carboxamide;
N-(2,3-dichlorobenzyl)-4-(1H-pyrrolo[2,3-b]pyridin-3-yl)-3,6-dihydropyridine-1(2H)-carboxamide;
N-(3,5-dichlorobenzyl)-4-(1H-pyrrolo[2,3-b]pyridin-3-yl)-3,6-dihydropyridine-1(2H)-carboxamide;
N-(2-cyclohex-1-en-1-ylethyl)-4-(1H-pyrrolo[2,3-b]pyridin-3-yl)-3,6-dihydropyridine-1(2H)-carboxamide;
N-(3,3-diphenylpropyl)-4-(1H-pyrrolo[2,3-b]pyridin-3-yl)-3,6-dihydropyridine-1(2H)-carboxamide;
N-[2-(1H-indol-3-yl)ethyl]-4-(1H-pyrrolo[2,3-b]pyridin-3-yl)-3,6-dihydropyridine-1(2H)-carboxamide;
4-(1H-pyrrolo[2,3-b]pyridin-3-yl)-N-(thien-2-ylmethyl)-3,6-dihydropyridine-1(2H)-carboxamide;
3-[1-(3-pyridin-3-yl-1,2,4-oxadiazol-5-yl)-1,2,3,6-tetrahydropyridin-4-yl]-1H-pyrrolo[2,3-b]pyridine;
N-[(1R)-1-(3-methoxyphenyl)ethyl]-4-(1H-pyrrolo[2,3-b]pyridin-3-yl)-3,6-dihydropyridine-1(2H)-carboxamide;
N-[(1R)-1-(3-methoxyphenyl)ethyl]-4-(1H-pyrrolo[2,3-b]pyridin-4-yl)-3,6-dihydropyridine-1(2H)-carboxamide; and
N-[(1R)-1-(3-methoxyphenyl)ethyl]-4-(1H-pyrrolo[2,3-b]pyridin-5-yl)-3,6-dihydropyridine-1(2H)-carboxamide.

18. A pharmaceutical composition comprising a therapeutically effective amount of a compound of claim (I) or pharmaceutically acceptable salt or solvate thereof, in combination with a pharmaceutically acceptable carrier.

19. A method for treating a disorder susceptible to treatment with ROCK modulators, said method comprising administering therapeutically effective amount of at least one compound of claim 1 or pharmaceutically acceptable salt or solvate thereof, to a subject in need thereof.

20. A method for treating a disease or a disorder in a mammal in need thereof comprising administering to the mammal therapeutically effective amount of at least one compound of claim 1 or a pharmaceutically acceptable salt thereof, wherein said disease or disorder is selected from the group consisting of hypertension, chronic and congestive heart failure, cardiac hypertrophy, chronic renal failure, cerebral vasospasm, pulmonary hypertension, ocular hypertension, cancer, tumor metastasis, asthma, male erectile dysfunctions, female sexual dysfunctions, over-active bladder syndrome, preterm labor, restenosis, atherosclerosis, neuronal injury, spinal cord injuries, traumatic brain injury and stroke, Parkinson's disease, Alzheimer disease, Huntington's disease, spinal muscular atrophy, amyotrophic lateral sclerosis, multiple sclerosis, encephalomyelitis, pain, rheumatoid arthritis, osteoarthritis, osteoporosis, irritable bowel syndrome, inflammatory bowel disease, HIV-1 encephalitis, diabetes, insulin resistance, ischemic CNS disorders, vascular or AD type dementia, glaucoma, psoriasis, retinopathy, benign prostatic hypertrophy, psychiatric disorders, depression, schizophrenia, obsessive compulsive disorder, bipolar disorder, epilepsy and seizure disorders, ischemia-reperfusion injury, myocardial infarct size and myocardial fibrosis, and diseases caused by viral and bacterial infections.

21. The method of claim 20 wherein the disease or disorder is selected from the group consisting of pain, asthma, cognitive dysfunctions, multiple sclerosis, cancer, rheumatoid arthritis, and spinal cord injuries.
